(19) United States
(12) Patent Application Publication
(10) Pub. No.: US 2025/0136677 A1
(43) Pub. Date: May 1, 2025

(54) METHOD OF IMPROVING THE CONSISTENCY AND/OR QUALITY OF A FELINE ANTIBODY

(71) Applicant: Zoetis Services LLC, Parsippany, NJ (US)

(72) Inventors: Gary Francis Bammert, Portage, MI (US); Steven Alan Dunham, Kalamazoo, MI (US); Sebastian C. J. Steiniger, Kalamazoo, MI (US)

(21) Appl. No.: 18/798,132

(22) Filed: Aug. 8, 2024

Related U.S. Application Data
(63) Continuation of application No. 17/571,117, filed on Jan. 7, 2022, now Pat. No. 12,084,494.
Application No. 17/571,117 is a continuation of application No. 16/256,223, filed on Jan. 24, 2019, now Pat. No. 11,530,262.
(60) Provisional application No. 62/643,940, filed on Mar. 16, 2018.

(51) Int. Cl. C07K 16/24; A61K 39/00; A61P 17/00; A61P 35/00; A61P 37/00; G01N 33/68

(52) U.S. Cl. CPC C07K 16/244; A61P 17/00; A61P 35/00; A61P 37/00; G01N 33/6869; A61K 2039/505; A61K 2039/552; C07K 2317/24; C07K 2317/33; C07K 2317/34; C07K 2317/76; C07K 2317/92; G01N 2333/54

(57) ABSTRACT

A method of improving the consistency and/or quality of a feline antibody is provided. The method includes expressing nucleotide sequence encoding a feline IgG kappa light chain and nucleotide sequence encoding a feline IgG heavy chain in a host cell to produce a feline antibody, wherein the nucleotide sequence encoding the feline IgG kappa light chain comprises a kappa light chain constant nucleotide sequence in which sequence encoding a C-terminal QRE sequence otherwise present in a wild-type feline IgG kappa light chain constant region is absent.

### CROSS-REFERENCE TO RELATED APPLICATIONS

This application is a continuation application of U.S. application Ser. No. 17/571,117, filed Jan. 7, 2022, which is a continuation application of U.S. application Ser. No. 16/256,223, filed Jan. 24, 2019, now U.S. Pat. No. 11,530,262, which claims priority to and the benefit of U.S. Provisional Application No. 62/643,940, filed Mar. 16, 2018, the entire contents each of which are incorporated herein by reference in their entirety.

### REFERENCE TO AN ELECTRONIC SEQUENCE LISTING

The contents of the electronic sequence listing (ZP000226C.xml; Size: 286,456 bytes; and Date of Creation: Jul. 30, 2024) is herein incorporated by reference in its entirety.

### FIELD OF THE INVENTION

The present invention relates to the field of recombinant monoclonal antibodies and their uses in clinical and scientific procedures, including diagnostic procedures. The present invention also provides isolated anti-IL31 antibodies in the form of veterinary compositions useful for treating an IL-31-mediated disorder in a mammal, such as a cat, dog, or horse. Methods of improving the consistency and/or quality of these antibodies are also provided.

### BACKGROUND OF THE INVENTION

Atopic dermatitis has been defined by the American College of Veterinary Dermatology task force as “a genetically-predisposed inflammatory and pruritic allergic skin disease with characteristic clinical features” (Olivry, et al. Veterinary Immunology and Immunopathology 2001; 81: 143-146). The task force also recognized that the disease in canines has been associated with allergen-specific IgE (Olivry, et al. 2001 supra; Marsella & Olivry Clinics in Dermatology 2003; 21: 122-133). Severe pruritus, along with secondary alopecia and erythema, are the most noticeable and concerning symptoms to pet owners.

The potential factors involved in allergic dermatitis are numerous and poorly understood. Components in food may trigger atopic dermatitis (Picco, et al. Vet Dermatol. 2008; 19: 150-155), as well as environmental allergens such as fleas, dust mites, ragweed, plant extracts, etc. Genetic factors also play an important role. Although there is no confirmed breed predilection, some mode of inheritance is thought to increase predisposition to atopic dermatitis (Sousa & Marsella Veterinary Immunology and Immunopathology 2001; 81: 153-157; Schwartzman, et al. Clin. Exp. Immunol. 1971; 9: 549-569).

The prevalence of atopic dermatitis is estimated to be 10% of the total canine population (Marsella & Olivry 2003 supra; Scott, et al. Canadian Veterinary Journal 2002; 43: 601-603; Hillier Veterinary Immunology and Immunopathology 2001; 81: 147-151). Globally, about 4.5 million dogs are affected with this chronic and lifelong condition. Incidence appears to be increasing. Canine breed and sex predilections have been suspected, but may vary greatly depending on geographical region (Hillier, 2001 supra; Picco, et al. 2008 supra).

Feline allergic dermatitis is an inflammatory and pruritic skin condition thought to be caused by an abnormal response of the immune system to substances that do not induce a reaction in healthy cats. The most consistent feature of feline allergic dermatitis is chronic recurrent pruritus. Common clinical presentations of allergic dermatitis in cats include self-induced alopecia, miliary dermatitis, eosinophilic granuloma complex lesions (including plaques, granulomas, and indolent ulcer), and focused head and neck pruritus characterized by excoriations, erosions, and/or ulcers. Breed and sex predilections have not been demonstrated and young cats seem more prone to the disease (Hobi et al. Vet Dermatol 2011 22: 406-413; Ravens et al. Vet Dermatol 2014; 25: 95-102; Buckely In Practice 2017; 39: 242-254).

Current treatments for cats diagnosed with allergic dermatitis depend on the severity of the clinical signs, duration, and owner preferences and include allergen-specific immunotherapy and antipruritic drugs such as glucocorticoids and cyclosporines (Buckley, supra). Immunotherapy treatment is effective for some patients but requires frequent injections, and clinical improvement may not be seen for 6-9 months (Buckley, supra). Immunosuppressive drugs like glucocorticoids and cyclosporines are generally effective however long term use often results in undesirable adverse effects.

Atopic dermatitis in horses is recognized as a potential cause of pruritus. The role of environmental allergens in equine atopic dermatitis is becoming better appreciated. The disease may be seasonal or non-seasonal, depending on the allergen(s) involved. Age, breed, and sex predilections have not been extensively reported. In preliminary work at the School of Veterinary Medicine, University of California, Davis (SVM-UCD), the median age at onset was 6.5 years, Thoroughbreds were the most common breed, accounting for 25% of the horses, and males (usually geldings) were almost twice as prevalent as mares; however, these data are from only 24 horses, and have not yet been compared with the hospital population at large. Pruritus, often directed against the face, distal legs, or trunk, is the most common clinical sign of equine atopic dermatitis. Alopecia, erythema, urticaria, and papules may all be present. Urticarial lesions may be quite severe, yet nonpruritic. There may be a familial predisposition for urticarial atopic dermatitis in the horse. Horses may have a secondary pyoderma, typified by excess scaling, small epidermal collarettes, or encrusted papules (“miliary dermatitis”). Diagnosis of atopic dermatitis is based on clinical signs and the exclusion of other diagnoses, especially insect (Culicoides) hypersensitivity (White Clin Tech Equine Pract 2005; 4: 311-313; Fadok Vet Clin Equine 2013; 29 541-550). Currently, management of atopic dermatitis in horses is done both symptomatically, by suppressing the inflammation and the pruritus triggered by the allergic response, and by addressing the specific cause (i.e., by identifying the responsible allergens and by formulating an allergen-specific vaccine). The symptomatic approach is typically needed in the short term to make the patient comfortable and minimize self-trauma. This approach relies on the use of a combination of topical and systemic therapies including antihistamines, essential fatty acids, pentoxifylline, and glucocorticoids. The primary approach to environmental allergy control involves the identification of allergens that trigger the hypersensitivity reaction. It is commonly accepted by dermatologists that allergen-specific immunotherapy can be of help to atopic horses. However, as a general rule, most horses show improvement only after the first 6 months of immunotherapy (Marsella Vet Clin Equine 2013; 29: 551-557). Also, long term use of immunosuppressive drugs in horses can result in undesirable adverse effects.

Interleukin-31 (IL-31), a cytokine produced by T helper type 2 cells, has been shown to induce pruritus in humans, mice, and dogs (Bieber N Engl J Med 2008; 358: 1483-1494; Dillon et al. Nat Immunol 2004; 5:752-60; U.S. Pat. No. 8,790,651 to Bammert et al.; Gonzalez et al. Vet Dermatl. 2013; 24(1): 48-53). IL-31 binds a co-receptor composed of IL-31 receptor A (IL-31RA) and the oncostatin M receptor (OSMR) (Dillon et al. 2004 supra and Bilsborough et al. J Allergy Clin Immunol. 2006 117(2):418-25). Receptor activation results in phosphorylation of STAT through JAK receptor(s). Expression of the co-receptor has been shown in macrophages, keratinocytes and in dorsal root ganglia.

Recently, it has been found that IL-31 is involved in dermatitis, pruritic skin lesions, allergy, and airway hypersensitivity. Cytopoint®, a canine anti-IL-31 monoclonal antibody produced by Zoetis Inc., Parsippany, NJ), has been shown to reduce pruritus and skin lesions in dogs with atopic dermatitis (Gonzalez et al. 2013 supra, Michels et al. Vet Dermatol. 2016; December; 27(6): 478-e129). It would be desirable to provide further anti-IL31 antibodies to prevent and treat IL-31-mediated disorders in veterinary mammals. Considering the currently unmet need for safe and effective alternative treatments for atopic and allergic dermatoses in cats and horses, it would be especially desirable to provide feline and equine anti-IL-31 antibodies to reduce pruritus and skin lesions in cats and horses with atopic dermatitis. It would also be desirable to improve the consistency and/or quality of such antibodies.

### SUMMARY OF THE INVENTION

The present provides a method of improving the consistency and/or quality of a feline antibody. The method includes expressing nucleotide sequence encoding a feline IgG kappa light chain and nucleotide sequence encoding a feline IgG heavy chain in a host cell to produce a feline antibody, wherein the nucleotide sequence encoding the feline IgG kappa light chain comprises a kappa light chain constant nucleotide sequence in which sequence encoding a C-terminal QRE sequence otherwise present in a wild-type feline IgG kappa light chain constant region is absent. In one embodiment, the C-terminal QRE sequence is deleted altogether or otherwise modified. In one embodiment, in place of the absent QRE sequence, additions of one or more amino acids to the c-terminus occur.

In one embodiment, the method of improving the consistency and/or quality of a feline antibody includes:
a) providing nucleotide sequence encoding a wild-type feline IgG kappa light chain constant region of a feline antibody, wherein said wild-type feline kappa light chain constant region comprises a C-terminal amino acid sequence of QRE;
b) removing sequence encoding the C-terminal QRE in the nucleotide sequence in a) to form a revised kappa light chain constant nucleotide sequence;
c) combining the revised kappa light chain constant nucleotide sequence from b) with nucleotide sequence encoding a feline IgG kappa light chain variable region to form nucleotide sequence encoding a complete feline IgG kappa light chain; and
d) expressing the nucleotide sequence encoding the complete feline IgG kappa light chain from c) and nucleotide sequence encoding a feline IgG heavy chain in a host cell to produce a feline antibody in which the C-terminal QRE sequence which would otherwise be present in the wild-type feline IgG kappa light chain constant region is absent. In one embodiment, in place of the absent QRE sequence, additions of one or more amino acids to the c-terminus occur.

In one embodiment, the nucleotide sequence encoding the feline IgG kappa light chain comprises a kappa light chain constant nucleotide sequence in which sequence encoding the absent C-terminal QRE sequence is substituted with up to three contiguous amino acids which have a minimal electrostatic charge influence.

In one embodiment, improving the consistency and/or quality of the feline antibody includes reducing the levels of free IgG kappa light chain, thereby increasing the percentage of intact feline IgG antibody monomer.

In one embodiment, the nucleotide sequence encoding the feline IgG kappa light chain and the nucleotide sequence encoding the feline IgG heavy chain are carried on the same vector used to transform the host cell. In another embodiment, the nucleotide sequence encoding the feline IgG kappa light chain and the nucleotide sequence encoding the feline IgG heavy chain are carried on separate vectors used to transform the host cell.

In on embodiment of the method of improving the consistency and/or quality of a feline antibody, the feline antibody specifically binds to a target involved in a cytokine and/or growth factor-mediated disorder. In one specific embodiment, the feline antibody specifically binds to feline IL-31. In another embodiment, the feline antibody binds to feline NGF.

In one embodiment, the feline antibody comprises a kappa light chain constant region having the sequence: RSDAQPSVFLFQPSLDELHTGSASIVCILNDFYPKEVNVKWKVDGVVQNKGIQESTTEQNSKD STYSLSSTLTMSSTEYQSHEKFSCEVTHKSLASTLVKSFQRSEC (SEQ ID NO: 186) or a variant thereof. Such variants can include, for example, an addition or modification of one or more amino acid residue(s) to the c-terminus of SEQ ID NO: 186. In one embodiment, up to three contiguous amino acids which have a minimal electrostatic charge influence are added to the C-terminal Cysteine at position 107 of SEQ ID NO: 186.

The revised kappa light chain constant regions described herein can be used in conjunction with any number of feline antibodies, such as including, but not limited to, any of the feline antibodies described in the specification and claims of this invention. Feline antibodies having targets other than IL-31 or NGF are also envisioned to be suitably combined with the revised kappa light chain constant regions disclosed herein. The present invention includes any feline antibody comprising such revised kappa light chain constant regions disclosed herein as such antibodies are reasonably expected to have improved consistency and/or quality on the basis of the disclosure in the instant specification and claims.

### BRIEF DESCRIPTION OF THE DRAWINGS

FIG. 1A is an alignment showing amino acid sequence conservation between IL-31 from different species. In particular, a comparison between SEQ ID NO: 155 (canine IL-31), SEQ ID NO: 157 (feline IL-31), SEQ ID NO: 165 (equine IL-31), and SEQ ID NO: 181 (human IL-31) is shown. In FIG. 1B, the percent amino acid sequence identity between canine, feline, horse and human IL-31 is also indicated.

FIG. 2 details the affinity with which candidate antibodies with CDRs derived from mouse origin bind feline and canine IL-31 using surface plasmon resonance (SPR) on a Biacore system (Biacore Life Sciences (GE Healthcare), Uppsala, Sweden).

FIG. 3 is a table showing potency (IC50 (μg/ml)) of candidate antibodies with CDRs derived from mouse origin as measured by canine and feline cellular assays. In particular, the candidate antibodies were assessed for their ability to inhibit IL-31-mediated STAT phosphorylation in canine DH-82 or feline FCWF4 macrophage-like cells.

FIG. 4 shows the results obtained for binding of candidate monoclonal antibodies with CDRs of dog origin to various proteins using both an indirect ELISA and Biacore methods. For the indirect ELISA, binding (ELISA OD) to wildtype feline IL-31 and a feline IL-31 15H05 mutant which had mutations in the monoclonal antibody 15H05 epitope region was assessed. To confirm binding, biacore analysis was performed using canine, feline, equine, human, the feline 15H05 mutant, and feline 11E12 mutant IL-31 proteins as surfaces and a single test concentration of antibody. The feline IL-31 11E12 mutant had mutations in the monoclonal antibody 11E12 epitope region.

FIG. 5A shows an alignment of mouse antibody 11E12 VL sequence (SEQ ID NO: 73) comparing previously disclosed caninized 11E12 sequences designated as Can_11E12_VL_cUn_1 (SEQ ID NO: 182) and CAN_11E12_VL_cUn_FW2 (SEQ ID NO: 184) to the felinized versions designated as FEL_11E12_VL1 (SEQ ID NO: 113) and FEL_11E12_VL1_FW2 (SEQ ID NO: 117). Noted below the alignment in FIG. 5A are dots showing the positons of relevant changes to Fel_11E12_VL1 that were necessary to restore affinity of this antibody to the IL-31 protein. FIG. 5B shows an alignment of the mouse antibody 15H05 VL sequence designated herein as MU_15H05_VL (SEQ ID NO: 69) with the felinized 15H05 VL sequences designated herein as FEl_15H05_VL1 (SEQ ID NO: 127) and FEl_15H05_VL_FW2 (SEQ ID NO: 135). The dots below the alignment in FIG. 5B indicate the necessary changes to the felinized 15H05 VL (Fel_15H05_VL1) that were required to not only restore, but improve, its affinity to canine and feline IL-31 when compared to the mouse and chimeric forms of this antibody.

FIG. 6A shows the alignment of wildtype feline IL-31 (SEQ ID NO: 157) with mutants 15H05 (SEQ ID NO: 163) and 11E12 (SEQ ID NO: 161) highlighting the positions where the alanine substitutions occur. FIG. 6B shows the feline IL-31 homology model highlighting the positions of two amino acids involved with binding of antibodies 11E12 (site 1) and 15H05 (site 2). FIG. 6C is a graph showing the results obtained for binding of monoclonal antibodies 11E12 and 15H05 to wild-type feline IL-31 and to mutant IL-31 proteins 15H05 (SEQ ID NO: 163) and 11E12 (SEQ ID NO: 161) when the wild-type and these mutants are used as the coating antigens.

FIGS. 7A and 7B are of graphs showing competition binding assessments of mAbs 15H05 and 11E12 using Biacore. FIG. 7A shows the competition binding data for mouse 15H05 and 11E12 antibodies to canine IL-31. FIG. 7B shows the competition binding data for antibodies 15H05 and 11E12 on a feline IL-31 surface.

FIG. 8 is of a graph showing the results obtained for binding of the individual receptor subunits of OSMR and IL-31Ra to wild-type feline IL-31 and to mutant IL-31 proteins 15H05 (SEQ ID NO: 163) and 11E12 (SEQ ID NO: 161) when the wild-type and these mutants are used as the coating antigens.

FIG. 9 is of a graph showing the preliminary efficacy of mouse: feline 11E12 chimera, mouse: feline 15H05 chimera, and felinized 11E12 (Feline 11E12 1.1) in an IL-31 induced pruritus model in cats.

FIGS. 10A and 10B are of graphs showing the In vivo evaluation of the efficacy of a felinized15H05 anti IL-31 antibody termed ZTS-361 in a cat pruritus challenge model. FIG. 10A shows the baseline pre-challenge pruritic behavior for the T01 vehicle placebo and T02 antibody ZTS-361 groups from day −7 through day 28 with day zero being the day of antibody administration to group T02. FIG. 10B shows the efficacy of antibody ZTS-361 demonstrating a significant reduction in pruritus observed on days 7 (p<0.0001), 21 (p<0.0027), and 28 (p<0.0238) following IL-31 challenge when compared to vehicle placebo control.

FIG. 11A is of a graph showing the plasma levels of IL-31 in client owned animals among dogs with atopic and allergic dermatitis compared to normal laboratory FIG. 11B is of a graph showing the results of a recent study to determine serum IL-31 levels in cats with a presumptive diagnosis of allergic dermatitis (AD) from several different geographic regions in the USA. FIG. 11C is of a graph showing the pharmacokinetic profile of canine IL-31 in dogs following administration of a subcutaneous dose of 1.75 μg/kg canine IL-31.

FIG. 12 shows a 4-12% non-reducing SDS PAGE comparing lane 1 which is ZTS-361, the heavy chain for which is (SEQ ID NO: 121; FEL_15H05_VH1) combined with feline IgG heavy chain constant (SEQ ID NO: 173; Feline_HC_AlleleA_1). For ZTS-361, the light chain is (SEQ ID NO: 135; FEL-15H05-VL1_FW2) combined with feline IgG light chain constant (SEQ ID NO: 175; Feline_LC_Kappa_G_minus). Lane 2 is mouse 15H05, the heavy chain for which is (SEQ ID NO: 67; MU_15H05_VH) combined with mouse IgG heavy chain constant (SEQ ID NO: 188; Mouse_HC_IgG1). For mouse 15H05, the light chain is (SEQ ID NO: 69; F MU_15H05_VL) combined with mouse IgG light chain constant (SEQ ID NO: 190; Mouse_LC_Kappa). Intact refers to an IgG with two heavy chains and two light chains held together by interchain disulfide bonds with an expected molecular weight of ˜150 kDa. HHL refers to “Heavy Heavy Light” and is an IgG with one light chain missing and an expected molecular weight of ˜125 kDa. HH refers to “Heavy Heavy” and is an IgG with both light chains missing and an expected molecular weight of ˜100 kDa. HL refers to “Heavy Light” and is an IgG with one heavy and one light chain and an expected molecular weight of ˜75 kDa. L refers to “Light” and is an IgG with one light chain and an expected molecular weight of ˜25 kDa, also referred to herein as free light chain.

FIG. 13A shows the results for non-reducing capillary gel electrophoresis (NR-CGE) comparing IgG from stable cell lines expressing ZTS-361 or mouse 15H05 (each described above). The percent monomer and subspecies are calculated from the experimental output using NR-CGE shown as the electropherograms in FIG. 13B. The time corrected area (TCA) is defined as the individual peak area from the instrument output divided by the migration time. Total TCA is defined as the sum of the TCAs for all peaks greater than or equal to 0.3%. Percent monomer intact IgG (% Monomer) and individual fragments (% HHL and % L) are calculated based on their individual TCAs as a percent of the total TCA. % Fragments are the sum of all the peak areas migrating with a lower molecular weight than that of intact IgG.

FIG. 14A shows a 4-12% non-reducing SDS PAGE comparing IgG from individual stable CHO clones of ZTS-361 (described above and in section 1.9 of example section) Lanes 1 and 8 are an IgG reference standard for comparison. Percent monomer is calculated from densiometric analysis of each band migrating to an expected molecular weight of ˜150 kDa using BioRad VersaDoc software. % Fragments are the sum of the individual bands with lower molecular weights. FIG. 14B Shows the results for non-reducing capillary gel electrophoresis (NR-CGE) comparing IgG from individual stable CHO clones of ZTS-361. The time corrected area (TCA) is defined as the individual peak area from the instrument output divided by the migration time. Total TCA is defined as the sum of the TCAs for all peaks greater than or equal to 0.3%. Percent monomer intact IgG (% Monomer) and percent fragments (% Fragments) are calculated based on their individual TCAs as a percent of the total TCA. % Fragments are the sum of all the peak areas migrating with a lower molecular weight than that of intact IgG.

FIG. 15 shows the amino acids on the c-terminal end of the Ig kappa light chain constant protein for the depicted species. Canine LC kappa wt, the depicted C-terminal amino acid residues are positions 103 to 109 of SEQ ID NO: 194, with the depicted nucleotide residue nos. being residue nos. 307 to 330 of SEQ ID NO: 195; Feline LC kappa G minus (G-), the depicted C-terminal amino acid residues are positions 105 to 110 of SEQ ID NO: 175, with the depicted nucleotide residue nos. being residue nos. 313 to 330 of SEQ ID NO: 176; Pig LC kappa, the depicted C-terminal amino acid residues are positions 104 to 108 of SEQ ID NO: 196, with the depicted nucleotide residue nos. being residue nos. 310 to 327 of SEQ ID NO: 197; Mink LC kappa, the depicted C-terminal amino acid residues are positions 105 to 108 of SEQ ID NO: 198, with the depicted nucleotide residue nos. being residue nos. 313 to 327 of SEQ ID NO: 199; Human LC kappa, the depicted C-terminal amino acid residues are positions 102 to 106 of SEQ ID NO: 192, with the depicted nucleotide residue nos. being residue nos. 304 to 321 of SEQ ID NO: 193; Mouse LC kappa, the depicted C-terminal amino acid residues are positions 102 to 106 of SEQ ID NO: 190, with the depicted nucleotide residue nos. being residue nos. 304 to 321 of SEQ ID NO: 191. The grey rectangle outlines the position of the c-terminal cysteine that forms an interchain disulphide bond with the IgG heavy constant chain necessary for an intact antibody. The grey triangle highlights the increasing percentage of kappa light chain utilization by IgGs from the species depicted according to; Canine, Feline, and Pig (Arun et al. 1996 Zentralbl Veterinarmed. November; 43(9):573-6), Mink (Bovkun et al. 1993 Eur J Immunol. August; 23(8):1929-34), Mouse (Woloschak et al. 1987 Mol Immunol. July; 24(7):751-7), Human (Barandun et al. 1976 Blood. January; 47(1):79-89). Nucleotides depicted in light grey highlight those codons in which a single nucleotide change at that position will result in a stop codon.

FIG. 16A is a pictoral representation of a feline IgG highlighting relative positions of the expected inter and intra-disulphide bonds. CYS15, the depicted amino acid residue is position 15 of Feline HC AlleleA wt (SEQ ID NO: 171) and Feline HC AlleleA 1 (SEQ ID NO: 173), with the depicted nucleotide residue nos. being residue nos. 43-45 of Feline HC AlleleA wt (SEQ ID NO: 172) and 43-45 of Feline HC AlleleA 1 (SEQ ID NO: 174) respectively. CYS107, the depicted amino acid residue is position 107 of Feline LC Kappa G minus (SEQ ID NO: 175), with the depicted nucleotide residue nos. being residue nos. 319-321 of Feline LC Kappa G minus (SEQ ID NO: 176). FIG. 16B is a homology model of ZTS-361 highlighting the positions of CYS15 and CYS107 described above. FIG. 16C is an enlarged picture of the area encircled in 16B again highlighting the positions of the two cysteines responsible for interchain pairing of feline heavy and light chains. The wire surface shells depicted are the calculated electrostatic contributions for the kappa light chain constant residues QRE that immediately follow CYS107 described in FIG. 15 for the feline LC kappa G-.

FIG. 17A describes the sequence ID numbers corresponding to the heavy and light chains used to create stable CHO cell lines producing antibodies ZTS-361 and ZTS-1505 (described above and/or in section 1.9 of example section). Highlighted is feline LC Kappa G minus QRE minus (SEQ ID NO: 186), the corresponding nucleotide sequence for which is feline LC Kappa G minus QRE minus (SEQ ID NO: 187). FIG. 17B shows the results for non-reducing capillary gel electrophoresis (NR-CGE) comparing IgG from individual stable CHO clones of ZTS-1505. The time corrected area (TCA) is defined as the individual peak area from the instrument output divided by the migration time. Total TCA is defined as the sum of the TCAs for all peaks greater than or equal to 0.3%. Percent monomer intact IgG (% Monomer) and percent fragments (% Fragments) are calculated based on their individual TCAs as a percent of the total TCA. % Fragments are the sum of all the peak areas migrating with a lower molecular weight than that of intact IgG. FIG. 17C shows the comparison of a single stable CHO clone producing antibody ZTS-361 compared to a single stable CHO clone producing ZTS-1505. Comparison of the two stable clones was made across 8 independent culture conditions labeled A through G. The percent viability of each culture following 14 days of culture is indicated. The titer indicates the amount of antibody produced after 14 days of culture by each stable clone under the respective culture condition. The percent monomer calculated from NR-CGE from each clone grown using various culture conditions is indicated.

FIG. 18A shows the percent identity comparing the variable regions of these anti feline NGF antibodies to anti IL-31 calculated using the ClustallW software. FIGS. 18B and 18C show the alignment of the anti feline IL-31 and NGF antibodies variable heavy and light chains respectively with the CDRs outlined with boxes.

FIG. 19 shows the results from the NR CGE comparing anti feline IL-31 and anti feline NGF antibodies with and without the modified kappa constant C-terminus.

FIG. 20 shows a ClustallW sequence alignment of feline to equine IL-31.

FIGS. 21A and 21B show an alignment of the variable heavy (FIG. 21A) and light (FIG. 21B) chains of antibodies 04H07 and 06A09 compared to mouse antibody 15H05 using ClustallW. For comparison, the location of each of the six CDRs are outlined with boxes.

FIG. 22 is of a Biacore Sensorgram showing the average profile of anti-IL31 antibody ZTS-1505+/−3 standard deviations used to define a threshold of response for screening of Alanine substituted CDR mutants.

FIG. 23 shows the results of Alanine substitution mutagenesis of the heavy chain CDRs of antibody ZTS-1505 comparing bindingi and IL-31 mediated pSTAT signalling inhibition to the wildtype antibody.

FIG. 24 shows the results of Alanine substitution mutagenesis of the light chain CDRs of antibody ZTS-1505 comparing bindingi and IL-31 mediated pSTAT signalling inhibition to the wildtype antibody.

FIGS. 25A and 25B show the binding affinity and cellular potency, respectively, of the two felinized antibodies designated herein as ZTS-5864 and ZTS-5865.

FIG. 26 is a graph depicted the results of an in vivo evaluation of the efficacy of the felinized ZTS-5864 anti-IL-31 antibody in a cat pruritus challenge model.

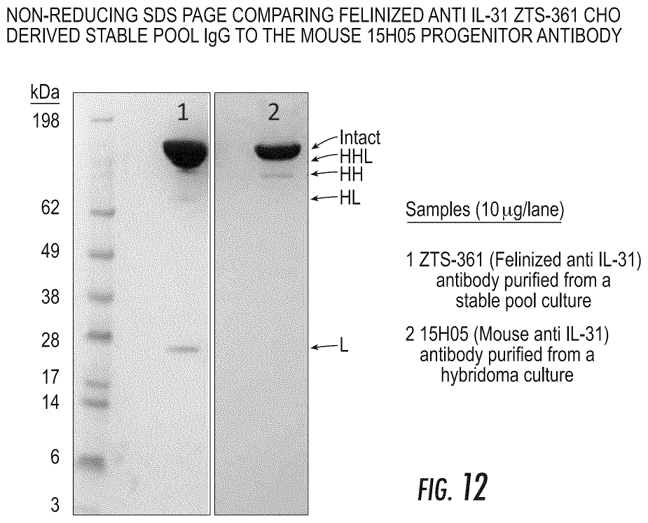

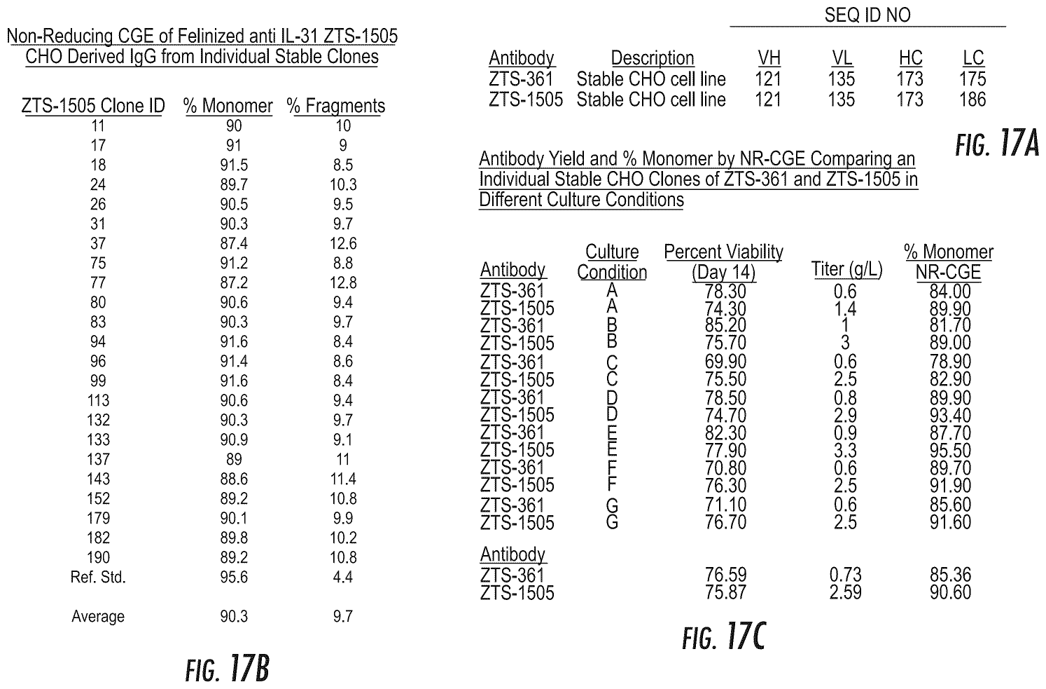

Antibodies described in FIGS. 12, 13A and 13B, and 14A and 14B were grown in culture conditions equivalent to culture condition A from FIG. 17C.

### BRIEF DESCRIPTION OF THE SEQUENCES

SEQ ID NO: 1 is a variable heavy chain CDR1 referred to herein as MU_15H05_VH_CDR1;
SEQ ID NO: 2 is a variable heavy chain CDR2 referred to herein as MU_15H05_VH_CDR2;
SEQ ID NO: 3 is a variable heavy chain CDR3 referred to herein as MU_15H05_VH_CDR3;
SEQ ID NO: 4 is a variable light CDR1 referred to herein as MU_15H05_VL_CDR1;
SEQ ID NO: 5 is a variable light CDR2 referred to herein as MU_15H05_VL_CDR2;
SEQ ID NO: 6 is a variable light CDR3 referred to herein as MU_15H05_VL_CDR3;
SEQ ID NO: 7 is a variable heavy chain CDR1 referred to herein as 11E12-VH-CDR1;
SEQ ID NO: 8 is a variable heavy chain CDR2 referred to herein as 11E12-VH-CDR2;
SEQ ID NO: 9 is a variable heavy chain CDR3 referred to herein as 11E12-VH-CDR3;
SEQ ID NO: 10 is a variable light chain CDR1 referred to herein as 11E12-VL-CDR1;
SEQ ID NO: 11 is a variable light chain CDR2 referred to herein as 11E12-VL-CDR2;
SEQ ID NO: 12 is a variable light chain CDR3 referred to herein as 11E12-VL-CDR3;
SEQ ID NO: 13 is a variable heavy chain CDR1 referred to herein as CAN_ZIL1_VH_CDR1;
SEQ ID NO: 14 is a variable heavy chain CDR2 referred to herein as CAN_ZIL1_VH_CDR2;
SEQ ID NO: 15 is a variable heavy chain CDR3 referred to herein as CAN_ZIL1_VH_CDR3;
SEQ ID NO: 16 is a variable light chain CDR1 referred to herein as CAN_ZIL1_VL_CDR1;
SEQ ID NO: 17 is a variable light chain CDR2 referred to herein as CAN_ZIL1_VL_CDR2;
SEQ ID NO: 18 is a variable light chain CDR3 referred to herein as CAN_ZIL1_VL_CDR3;
SEQ ID NO: 19 is a variable heavy chain CDR1 referred to herein as CAN_ZIL8_VH_CDR1;
SEQ ID NO: 20 is a variable heavy chain CDR2 referred to herein as CAN_ZIL8_VH_CDR2;
SEQ ID NO: 21 is a variable heavy chain CDR3 referred to herein as CAN_ZIL8_VH_CDR3;
SEQ ID NO: 22 is a variable light chain CDR1 referred to herein as CAN_ZIL8_VL_CDR1;
SEQ ID NO: 23 is a variable light chain CDR2 referred to herein as CAN_ZIL8_VL_CDR2;
SEQ ID NO: 24 is a variable light chain CDR3 referred to herein as CAN_ZIL8_VL_CDR3;
SEQ ID NO: 25 is a variable heavy chain CDR1 referred to herein as CAN_ZIL9_VH_CDR1;
SEQ ID NO: 26 is a variable heavy chain CDR2 referred to herein as CAN_ZIL9_VH_CDR2;
SEQ ID NO: 27 is a variable heavy chain CDR3 referred to herein as CAN_ZIL9_VH_CDR3;
SEQ ID NO: 28 is a variable light chain CDR1 referred to herein as CAN_ZIL9_VL_CDR1;
SEQ ID NO: 29 is a variable light chain CDR2 referred to herein as CAN_ZIL9_VL_CDR2;
SEQ ID NO: 30 is a variable light chain CDR3 referred to herein as CAN_ZIL9_VL_CDR3;
SEQ ID NO: 31 is a variable heavy chain CDR1 referred to herein as CAN_ZIL11_VH_CDR1;
SEQ ID NO: 32 is a variable heavy chain CDR2 referred to herein as CAN_ZIL11_VH_CDR2;
SEQ ID NO: 33 is a variable heavy chain CDR3 referred to herein as CAN_ZIL11_VH_CDR3;
SEQ ID NO: 34 is a variable light chain CDR1 referred to herein as CAN_ZIL11_VL_CDR1;
SEQ ID NO: 35 is a variable light chain CDR2 referred to herein as CAN_ZIL11_VL_CDR2;
SEQ ID NO: 36 is a variable light chain CDR3 referred to herein as CAN_ZIL11_VL_CDR3;
SEQ ID NO: 37 is a variable heavy chain CDR1 referred to herein as CAN_ZIL69_VH_CDR1;
SEQ ID NO: 38 is a variable heavy chain CDR2 referred to herein as CAN_ZIL69_VH_CDR2;
SEQ ID NO: 39 is a variable heavy chain CDR3 referred to herein as CAN_ZIL69_VH_CDR3;
SEQ ID NO: 40 is a variable light chain CDR1 referred to herein as CAN_ZIL69_VL_CDR1;
SEQ ID NO: 41 is a variable light chain CDR2 referred to herein as CAN_ZIL69_VL_CDR2;
SEQ ID NO: 42 is a variable light chain CDR3 referred to herein as CAN_ZIL69_VL_CDR3;
SEQ ID NO: 43 is a variable heavy chain CDR1 referred to herein as CAN_ZIL94_VH_CDR1;
SEQ ID NO: 44 is a variable heavy chain CDR2 referred to herein as CAN_ZIL94_VH_CDR2;
SEQ ID NO: 45 is a variable heavy chain CDR3 referred to herein as CAN_ZIL94_VH_CDR3;
SEQ ID NO: 46 is a variable light chain CDR1 referred to herein as CAN_ZIL94_VL_CDR1;
SEQ ID NO: 47 is a variable light chain CDR2 referred to herein as CAN_ZIL94_VL_CDR2;
SEQ ID NO: 48 is a variable light chain CDR3 referred to herein as CAN_ZIL94_VL_CDR3;
SEQ ID NO: 49 is a variable heavy chain CDR1 referred to herein as CAN_ZIL154_VH_CDR1;
SEQ ID NO: 50 is a variable heavy chain CDR2 referred to herein as CAN_ZIL154_VH_CDR2;
SEQ ID NO: 51 is a variable heavy chain CDR3 referred to herein as CAN_ZIL154_VH_CDR3;
SEQ ID NO: 52 is a variable light chain CDR1 referred to herein as CAN_ZIL154_VL_CDR1;
SEQ ID NO: 53 is a variable light chain CDR2 referred to herein as CAN_ZIL154_VL_CDR2;
SEQ ID NO: 54 is a variable light chain CDR3 referred to herein as CAN_ZIL154_VL_CDR3;
SEQ ID NO: 55 is a variable heavy chain CDR1 referred to herein as CAN_ZIL159_VH_CDR1;
SEQ ID NO: 56 is a variable heavy chain CDR2 referred to herein as CAN_ZIL159_VH_CDR2;
SEQ ID NO: 57 is a variable heavy chain CDR3 referred to herein as CAN_ZIL159_VH_CDR3;
SEQ ID NO: 58 is a variable light chain CDR1 referred to herein as CAN_ZIL159_VL_CDR1;
SEQ ID NO: 59 is a variable light chain CDR2 referred to herein as CAN_ZIL159_VL_CDR2;
SEQ ID NO: 60 is a variable light chain CDR3 referred to herein as CAN_ZIL159_VL_CDR3;
SEQ ID NO: 61 is a variable heavy chain CDR1 referred to herein as CAN_ZIL171_VH_CDR1;
SEQ ID NO: 62 is a variable heavy chain CDR2 referred to herein as CAN_ZIL171_VH_CDR2;
SEQ ID NO: 63 is a variable heavy chain CDR3 referred to herein as CAN_ZIL171_VH_CDR3;
SEQ ID NO: 64 is a variable light chain CDR1 referred to herein as CAN_ZIL171_VL_CDR1;
SEQ ID NO: 65 is a variable light chain CDR2 referred to herein as CAN_ZIL171_VL_CDR2;
SEQ ID NO: 66 is a variable light chain CDR3 referred to herein as CAN_ZIL171_VL_CDR3;
SEQ ID NO: 67 is a variable heavy chain referred to herein as MU_15H05_VH;
SEQ ID NO: 68 is a nucleotide sequence encoding the variable heavy chain referred to herein as MU_15H05_VH;
SEQ ID NO: 69 is a variable light chain referred to herein as MU_15H05_VL;
SEQ ID NO: 70 is a nucleotide sequence encoding the variable light chain referred to herein as MU_15H0_VL;
SEQ ID NO: 71 is a variable heavy chain referred to herein as MU-11E12-VH;
SEQ ID NO: 72 is a nucleotide sequence encoding the variable heavy chain referred to herein as MU-11E12-VH;
SEQ ID NO: 73 is a variable light chain referred to herein as MU-11E12-VL;
SEQ ID NO: 74 is a nucleotide sequence encoding the variable light chain referred to herein as MU-11E12-VL;
SEQ ID NO: 75 is a variable heavy chain referred to herein as CAN-ZIL1_VH;
SEQ ID NO: 76 is a nucleotide sequence encoding the variable heavy chain referred to herein as CAN-ZIL1_VH;
SEQ ID NO: 77 is a variable light chain referred to herein as CAN-ZIL1_VL;
SEQ ID NO: 78 is a nucleotide sequence encoding the variable light chain referred to herein as CAN-ZIL1_VL;
SEQ ID NO: 79 is a variable heavy chain referred to herein as CAN-ZIL8_VH;
SEQ ID NO: 80 is a nucleotide sequence encoding the variable heavy chain referred to herein as CAN-ZIL8_VH;
SEQ ID NO: 81 is a variable light chain referred to herein as CAN-ZIL8_VL;
SEQ ID NO: 82 is a nucleotide sequence encoding the variable light chain referred to herein as CAN-ZIL8_VL;
SEQ ID NO: 83 is a variable heavy chain referred to herein as CAN-ZIL9_VH;
SEQ ID NO: 84 is a nucleotide sequence encoding the variable heavy chain referred to herein as CAN-ZIL9_VH;
SEQ ID NO: 85 is a variable light chain referred to herein as CAN-ZIL9_VL;
SEQ ID NO: 86 is a nucleotide sequence encoding the variable light chain referred to herein as CAN-ZIL9_VL;
SEQ ID NO: 87 is a variable heavy chain referred to herein as CAN-ZIL11_VH;
SEQ ID NO: 88 is a nucleotide sequence encoding the variable heavy chain referred to herein as CAN-ZIL11_VH;
SEQ ID NO: 89 is a variable light chain referred to herein as CAN-ZIL11_VL;
SEQ ID NO: 90 is a nucleotide sequence encoding the variable light chain referred to herein as CAN-ZIL11_VL;
SEQ ID NO: 91 is a variable heavy chain referred to herein as CAN-ZIL69_VH;
SEQ ID NO: 92 is a nucleotide sequence encoding the variable heavy chain referred to herein as CAN-ZIL69_VH;
SEQ ID NO: 93 is a variable light chain referred to herein as CAN-ZIL69_VL;
SEQ ID NO: 94 is a nucleotide sequence encoding the variable light chain referred to herein as CAN-ZIL69_VL;
SEQ ID NO: 95 is a variable heavy chain referred to herein as CAN-ZIL94_VH;
SEQ ID NO: 96 is a nucleotide sequence encoding the variable heavy chain referred to herein as CAN-ZIL94_VH;
SEQ ID NO: 97 is a variable light chain referred to herein as CAN-ZIL94_VL;
SEQ ID NO: 98 is a nucleotide sequence encoding the variable light chain referred to herein as CAN-ZIL94_VL;
SEQ ID NO: 99 is a variable heavy chain referred to herein as CAN-ZIL154_VH;
SEQ ID NO: 100 is a nucleotide sequence encoding the variable heavy chain referred to herein as CAN-ZIL154_VH;
SEQ ID NO: 101 is a variable light chain referred to herein as CAN-ZIL154_VL;
SEQ ID NO: 102 is a nucleotide sequence encoding the variable light chain referred to herein as CAN-ZIL154_VL;
SEQ ID NO: 103 is a variable heavy chain referred to herein as CAN-ZIL159_VH;
SEQ ID NO: 104 is a nucleotide sequence encoding the variable heavy chain referred to herein as CAN-ZIL159_VH;
SEQ ID NO: 105 is a variable light chain referred to herein as CAN-ZIL159_VL;
SEQ ID NO: 106 is a nucleotide sequence encoding the variable light chain referred to herein as CAN-ZIL159_VL;
SEQ ID NO: 107 is a variable heavy chain referred to herein as CAN-ZIL171_VH;
SEQ ID NO: 108 is a nucleotide sequence encoding the variable heavy chain referred to herein as CAN-ZIL171_VH;
SEQ ID NO: 109 is a variable light chain referred to herein as CAN-ZIL171_VL;
SEQ ID NO: 110 is a nucleotide sequence encoding the variable light chain referred to herein as CAN-ZIL171_VL;
SEQ ID NO: 111 is a variable heavy chain referred to herein as FEL_11E12_VH1;
SEQ ID NO: 112 is a nucleotide sequence encoding the variable heavy chain referred to herein as FEL_11E12_VH1;
SEQ ID NO: 113 is a variable light chain referred to herein as FEL_11E12_VL1;
SEQ ID NO: 114 is a nucleotide sequence encoding the variable light chain referred to herein as FEL_11E12_VL1;
SEQ ID NO: 115 is a variable light chain referred to herein as FEL_11E12_VL2;
SEQ ID NO: 116 is a nucleotide sequence encoding the variable light chain referred to herein as FEL_11E12_VL2;
SEQ ID NO: 117 is a variable light chain referred to herein as FEL_11E12_VL1_FW2;
SEQ ID NO: 118 is a nucleotide sequence encoding the variable light chain referred to herein as FEL_11E12_VL1_FW2;
SEQ ID NO: 119 is a variable light chain referred to herein as FEL_11E12_VL1_K46Q;
SEQ ID NO: 120 is a nucleotide sequence encoding the variable light chain referred to herein as FEL_11E12_VL1_K46Q;
SEQ ID NO: 121 is a variable heavy chain referred to herein as FEL_15H05_VH1;
SEQ ID NO: 122 is a nucleotide sequence encoding the variable heavy chain referred to herein as FEL_15H05_VH1;
SEQ ID NO: 123 is a variable heavy chain referred to herein as FEL_15H05_VH2;
SEQ ID NO: 124 is a nucleotide sequence encoding the variable heavy chain referred to herein as FEL_15H05_VH2;
SEQ ID NO: 125 is a variable heavy chain referred to herein as FEL_15H05_VH3;
SEQ ID NO: 126 is a nucleotide sequence encoding the variable heavy chain referred to herein as FEL_15H05_VH3;
SEQ ID NO: 127 is a variable light chain referred to herein as FEL_15H05_VL1;
SEQ ID NO: 128 is a nucleotide sequence encoding the variable light chain referred to herein as FEL_15H05_VL1;
SEQ ID NO: 129 is a variable light chain referred to herein as FEL_15H05_VL2;
SEQ ID NO: 130 is a nucleotide sequence encoding the variable light chain referred to herein as FEL_15H05_VL2;
SEQ ID NO: 131 is a variable light chain referred to herein as FEL_15H05_VL3;
SEQ ID NO: 132 is a nucleotide sequence encoding the variable light chain referred to herein as FEL_15H05_VL3;
SEQ ID NO: 133 is a variable light chain referred to herein as FEL_15H05_VL1_FW1;
SEQ ID NO: 134 is a nucleotide sequence encoding the variable light chain referred to herein as FEL_15H05_VL1_FW1;
SEQ ID NO: 135 is a variable light chain referred to herein as FEL_15H05_VL1_FW2;
SEQ ID NO: 136 is a nucleotide sequence encoding the variable light chain referred to herein as FEL_15H05_VL1_FW2;
SEQ ID NO: 137 is a variable light chain referred to herein as FEL_15H05_VL1_FW3;
SEQ ID NO: 138 is a nucleotide sequence encoding the variable light chain referred to herein as FEL_15H05_VL1_FW3;
SEQ ID NO: 139 is a variable light chain referred to herein as FEL_15H05_VL1_FW1_FW2;
SEQ ID NO: 140 is a nucleotide sequence encoding the variable light chain referred to herein as FEL_15H05_VL1_FW1_FW2;
SEQ ID NO: 141 is a variable light chain referred to herein as FEL_15H05_VL1_FW1_FW3;
SEQ ID NO: 142 is a nucleotide sequence encoding the variable light chain referred to herein as FEL_15H05_VL1_FW1_FW3;
SEQ ID NO: 143 is a variable light chain referred to herein as FEL_15H05_VL1_FW2_FW3;
SEQ ID NO: 144 is a nucleotide sequence encoding the variable light chain referred to herein as FEL_15H05_VL1_FW2_FW3;
SEQ ID NO: 145 is a variable light chain referred to herein as FEL_15H05_VL1_FW2_K42N;
SEQ ID NO: 146 is a nucleotide sequence encoding the variable light chain referred to herein as FEL_15H05_VL1_FW2_K42N;
SEQ ID NO: 147 is a variable light chain referred to herein as FEL_15H05_VL1_FW2_V43I;
SEQ ID NO: 148 is a nucleotide sequence encoding the variable light chain referred to herein as FEL_15H05_VL1_FW2_V43I;
SEQ ID NO: 149 is a variable light chain referred to herein as FEL_15H05_VL1_FW2_L46V;
SEQ ID NO: 150 is a nucleotide sequence encoding the variable light chain referred to herein as FEL_15H05_VL1_FW2_L46V;
SEQ ID NO: 151 is a variable light chain referred to herein as FEL_15H05_VL1_FW2_Y49N;
SEQ ID NO: 152 is a nucleotide sequence encoding the variable light chain referred to herein as FEL_15H05_VL1_FW2_Y49N;
SEQ ID NO: 153 is a variable light chain referred to herein as FEL_15H05_VL1_FW2_K42N_V43I;
SEQ ID NO: 154 is a nucleotide sequence encoding the variable light chain referred to herein as FEL_15H05_VL1_FW2_K42N_V43I;
SEQ ID NO: 155 is the amino acid sequence of canine IL-31 protein referred to herein as Canine_IL31;
SEQ ID NO: 156 is the nucleotide sequence encoding the canine IL-31 protein referred to herein as Canine_IL31;
SEQ ID NO: 157 is an amino acid sequence referred to herein as Feline_IL_31_wildtype which represents wild-type feline IL-31 protein with a C-terminal His tag;
SEQ ID NO: 158 is a nucleotide sequence encoding the amino acid sequence referred to herein as Feline_IL31_wildtype;
SEQ ID NO: 159 is an amino acid sequence referred to herein as Feline_IL_31_E_coli which represents feline IL-31 protein with an N-terminal His tag;
SEQ ID NO: 160 is a nucleotide sequence encoding the amino acid sequence referred to herein as Feline_IL_31_E_coli;
SEQ ID NO: 161 is an amino acid sequence referred to herein as Feline_IL31_11E12_mutant which represents mutant Feline IL-31 11E12 protein with a C-terminal His tag;
SEQ ID NO: 162 is a nucleotide sequence encoding the amino acid sequence referred to herein as Feline_IL31_11E12_mutant;
SEQ ID NO: 163 is an amino acid sequence referred to herein as Feline_IL31_15H05_mutant which represents mutant Feline IL-31 15H05 protein with a C-terminal His tag;
SEQ ID NO: 164 is a nucleotide sequence encoding the amino acid sequence referred to herein as Feline_IL31_15H05_mutant;
SEQ ID NO: 165 is the amino acid of equine IL-31 protein referred to herein as Equine_IL31;
SEQ ID NO: 166 is the nucleotide sequence encoding the equine IL-31 protein referred to herein as Equine_IL31;
SEQ ID NO: 167 is an amino acid sequence referred to herein as Feline_OSMR_hIgG1_Fc which represents the extracellular domain of feline OSMR fused to human IgG1 Fc;
SEQ ID NO: 168 is a nucleotide sequence encoding the amino acid sequence referred to herein as Feline_OSMR_hIgG1_Fc;
SEQ ID NO: 169 is an amino acid sequence referred to herein as Feline_IL31Ra_HIgG1_Fc_X1_Fn3 which represents feline IL-31Ra fused to human IgG1 Fc;
SEQ ID NO: 170 is a nucleotide sequence encoding the amino acid sequence referred to herein as Feline_IL31Ra_HIgG1_Fc_X1_Fn3;
SEQ ID NO: 171 is a feline heavy chain referred to herein as Feline_HC_AlleleA_wt;
SEQ ID NO: 172 is a nucleotide sequence encoding the feline heavy chain referred to herein as Feline_HC_AlleleA_wt;
SEQ ID NO: 173 is a feline heavy chain referred to herein as Feline_HC_AlleleA_1, which was engineered to replace the M, L, and G at positions 120, 121, and 123, repectively, of the wild-type sequence of SEQ ID NO: 171 with Alanines (A) in order to elminate antibody effector function;
SEQ ID NO: 174 is a nucleotide sequence encoding the feline heavy chain referred to herein as Feline_HC_AlleleA_1;
SEQ ID NO: 175 is a feline kappa light chain referred to herein as Feline_LC_Kappa_G_minus, which was engineered with a glycosylation knockout (G-) at position 103 such that an N normally present in the wild-type feline kappa light chain at this position was changed to Q;
SEQ ID NO: 176 is a nucleotide sequence encoding the amino acid sequence of a feline kappa light chain referred to herein as Feline_LC_Kappa_G_minus;
SEQ ID NO: 177 is a canine heavy chain referred to herein as Canine_HC_65_1;
SEQ ID NO: 178 is a nucleotide sequence encoding the canine heavy chain referred to herein as Canine_HC_65_1;
SEQ ID NO: 179 is a canine kappa light chain referred to herein as Canine_LC_Kappa;
SEQ ID NO: 180 is a nucleotide sequence encoding the canine kappa light chain referred to herein as Canine_LC_Kappa;
SEQ ID NO: 181 is the amino acid sequence of human IL-31.
SEQ ID NO: 182 is a variable light chain mAb sequence referred to herein as Can_11E12_VL_cUn_1;
SEQ ID NO: 183 is a nucleotide sequence encoding the variable light chain mAb sequence referred to herein as Can_11E12_VL_cUn_1;
SEQ ID NO: 184 is a variable light chain mAb sequence referred to herein as Can_11E12_VL_cUn_FW2;
SEQ ID NO: 185 is a nucleotide sequence encoding the variable light chain mAb sequence referred to herein as Can_11E12_VL_cUn_FW2;
SEQ ID NO: 186 is a feline kappa light chain referred to herein as Feline_LC_Kappa_G_minus_QRE_minus, which was engineered with i) a glycosylation knockout (G-) at position 103 such that an N normally present in the wild-type feline kappa light chain at this position was changed to Q, and ii) a deletion of the C-terminus QRE relative to the wild-type;
SEQ ID NO: 187 is a nucleotide sequence encoding the feline kappa light chain referred to herein as Feline_LC_Kappa_G_minus_QRE_minus;
SEQ ID NO: 188 is a mouse heavy chain designated herein as Mouse_HC_IgG1;
SEQ ID NO: 189 is a nucleotide sequence encoding the mouse heavy chain designated herein as Mouse_HC_IgG1;
SEQ ID NO: 190 is a mouse kappa light chain designated herein as Mouse_LC_Kappa;
SEQ ID NO: 191 is a nucleotide sequence encoding the mouse kappa light chain designated herein as Mouse_LC_Kappa;
SEQ ID NO: 192 is a human kappa light chain designated herein as Human_LC_Kappa;
SEQ ID NO: 193 is a nucleotide sequence encoding the human kappa light chain designated herein as Human_LC_Kappa;
SEQ ID NO: 194 is a wild-type canine kappa light chain designated herein as Canine_LC_Kappa_wt;
SEQ ID NO: 195 is a nucleotide sequence encoding the wild-type canine kappa light chain designated herein as Canine_LC_Kappa_wt;
SEQ ID NO: 196 is a pig kappa light chain designated herein as Pig_LC_Kappa;
SEQ ID NO: 197 is a nucleotide sequence encoding the pig kappa light chain designated herein as Pig_LC_Kappa;
SEQ ID NO: 198 is a mink kappa light chain designated herein as Mink_LC_Kappa;
SEQ ID NO: 199 is a nucleotide sequence encoding the mink kappa light chain designated herein as Mink_LC_Kappa;
SEQ ID NO: 200 is a variable heavy chain CDR1 referred to herein as Mu_04H07_VH_CDR1;
SEQ ID NO: 201 is a variable heavy chain CDR2 referred to herein as Mu_04H07_VH_CDR2;
SEQ ID NO: 202 is a variable heavy chain CDR3 referred to herein as Mu_04H07_VH_CDR3;
SEQ ID NO: 203 is a variable light CDR1 referred to herein as Mu_04H07_VL_CDR1;
SEQ ID NO: 204 is a variable light CDR2 referred to herein as Mu_04H07_VL_CDR2;
SEQ ID NO: 205 is a variable light CDR3 referred to herein as Mu_04H07_VL_CDR3;
SEQ ID NO: 206 is a variable heavy chain CDR1 referred to herein as Mu_06A09_VH_CDR1;
SEQ ID NO: 207 is a variable heavy chain CDR2 referred to herein as Mu_06A09_VH_CDR2;
SEQ ID NO: 208 is a variable heavy chain CDR3 referred to herein as Mu_06A09_VH_CDR3;
SEQ ID NO: 209 is a variable light CDR1 referred to herein as Mu_06A09_VL_CDR1;
SEQ ID NO: 210 is a variable light CDR2 referred to herein as Mu_06A09_VL_CDR2;
SEQ ID NO: 211 is a variable light CDR3 referred to herein as Mu_06A09_VL_CDR3;
SEQ ID NO: 212 is a variable heavy chain referred to herein as Mu_04H07_VH;
SEQ ID NO: 213 is a nucleotide sequence encoding the variable heavy chain referred to herein as Mu_04H07_VH;
SEQ ID NO: 214 is a variable light chain referred to herein as Mu_04H07_VL;
SEQ ID NO: 215 is a nucleotide sequence encoding the variable light chain referred to herein as Mu_04H07_VL;
SEQ ID NO: 216 is a variable heavy chain referred to herein as Mu_06A09_VH;
SEQ ID NO: 217 is a nucleotide sequence encoding the variable heavy chain referred to herein as Mu_06A09_VH;
SEQ ID NO: 218 is a variable light chain referred to herein as Mu_06A09_VL;
SEQ ID NO: 219 is a nucleotide sequence encoding the variable light chain referred to herein as Mu_06A09_VL;
SEQ ID NO: 220 is a variable heavy chain referred to herein as ZTS_768_VH;
SEQ ID NO: 221 is a nucleotide sequence encoding the variable heavy chain referred to herein as ZTS_768_VH;
SEQ ID NO: 222 is a variable light chain referred to herein as ZTS_768_VL;
SEQ ID NO: 223 is a nucleotide sequence encoding the variable light chain referred to herein as ZTS_768_VL;
SEQ ID NO: 224 is a variable heavy chain referred to herein as ZTS_943_VH;
SEQ ID NO: 225 is a nucleotide sequence encoding the variable heavy chain referred to herein as ZTS_943_VH;
SEQ ID NO: 226 is a variable light chain referred to herein as ZTS_943_VL;
SEQ ID NO: 227 is a nucleotide sequence encoding the variable light chain referred to herein as ZTS_943_VL;
SEQ ID NO: 228 is a variable heavy chain referred to herein as ZTS_5864_VH;
SEQ ID NO: 229 is a nucleotide sequence encoding the variable heavy chain referred to herein as ZTS_5864_VH;
SEQ ID NO: 230 is a variable light chain referred to herein as ZTS_5864_VL;
SEQ ID NO: 231 is a nucleotide sequence encoding the variable light chain referred to herein as ZTS_5864_VL;
SEQ ID NO: 232 is a variable heavy chain referred to herein as ZTS_5865_VH;
SEQ ID NO: 233 is a nucleotide sequence encoding the variable heavy chain referred to herein as ZTS_5865_VH;
SEQ ID NO: 234 is a variable light chain referred to herein as ZTS_5865_VL;
SEQ ID NO: 235 is a nucleotide sequence encoding the variable light chain referred to herein as ZTS_5865_VL;
SEQ ID NO: 236 is the amino acid sequence of a feline kappa light chain referred to herein as Feline_LC_Lambda;
SEQ ID NO: 237 is a nucleotide sequence encoding the feline kappa light chain referred to herein as Feline_LC_Lambda.

### Definitions

Before describing the present invention in detail, several terms used in the context of the present invention will be defined. In addition to these terms, others are defined elsewhere in the specification, as necessary. Unless otherwise expressly defined herein, terms of art used in this specification will have their art-recognized meanings.

As used in the specification and claims, the singular form “a”, “an” and “the” include plural references unless the context clearly dictates otherwise. For example, reference to “an antibody” includes a plurality of such antibodies.

As used herein, the term “comprising” is intended to mean that the compositions and methods include the recited elements, but not excluding others.

Epitope, as used herein, refers to the antigenic determinant recognized by the CDRs of the antibody. In other words, epitope refers to that portion of any molecule capable of being recognized by, and bound by, an antibody. Unless indicated otherwise, the term “epitope” as used herein, refers to the region of IL-31 to which an anti-IL-31 agent is reactive to.

An “antigen” is a molecule or a portion of a molecule capable of being bound by an antibody which is additionally capable of being recognized by, and bound by, an antibody (the corresponding antibody binding region may be referred to as a paratope). In general, epitopes consist of chemically active surface groupings of molecules, for example, amino acids or sugar side chains, and have specific three-dimensional structural characteristics as well as specific charge characteristics. Epitopes are the antigenic determinant on a protein that is recognized by the immune system. The components of the immune system recognizing epitopes are antibodies, T-cells, and B-cells. T-cell epitopes are displayed on the surface of antigen-presenting cells (APCs) and are typically 8-11 (MHC class I) or 15 plus (MHC class II) amino acids in length. Recognition of the displayed MHC-peptide complex by T-cells is critical to their activation. These mechanisms allow for the appropriate recognition of self versus “non-self” proteins such as bacteria and viruses. Independent amino acid residues that are not necessarily contiguous contribute to interactions with the APC binding cleft and subsequent recognition by the T-Cell receptor (Janeway, Travers, Walport, Immunobiology: The Immune System in Health and Disease. 5^{th} edition New York: Garland Science; 2001). Epitopes that are recognized by soluble antibodies and cell surface associated B-cell receptors vary greatly in length and degree of continuity (Sivalingam and Shepherd, Immunol. 2012; 51(3-4): 304-309). Again even linear epitopes or epitopes found in a continuous stretch of protein sequence will often have discontiguous amino acids that represent the key points of contact with the antibody paratopes or B-cell receptor. Epitopes recognized by antibodies and B-cells can be conformational with amino acids comprising a common area of contact on the protein in three dimensional space and are dependent on tertiary and quaternary structural features of the protein. These residues are often found in spatially distinct areas of the primary amino acid sequence.

A “mimotope” as used herein is a linear or constrained peptide which mimics an antigen's epitope. A mimotope may have a primary amino acid sequence capable of eliciting a T-cell effector response and/or a three dimensional structure necessary to bind B-cells resulting in maturation of an acquired immunological response in an animal. An antibody for a given epitope antigen will recognize a mimotope which mimics that epitope.

The term “specifically” in the context of antibody binding, refers to high avidity and/or high affinity binding of an antibody to a specific antigen, i.e., a polypeptide, or epitope. In many embodiments, the specific antigen is an antigen (or a fragment or subfraction of an antigen) used to immunize the animal host from which the antibody-producing cells were isolated. Antibody specifically binding an antigen is stronger than binding of the same antibody to other antigens. Antibodies which bind specifically to a polypeptide may be capable of binding other polypeptides at a weak, yet detectable level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity with a K_{D} of 10⁻⁷ M or less, e.g., 10⁻³ M or less (e.g., 10⁻⁹ M or less, 10⁻¹⁰ or less, 10⁻¹¹ or less, 10⁻¹² or less, or 10⁻¹³ or less, etc.).

As used herein, the term “antibody” refers to an intact immunoglobulin having two light and two heavy chains. Thus a single isolated antibody or fragment may be a polyclonal antibody, a monoclonal antibody, a synthetic antibody, a recombinant antibody, a chimeric antibody, a heterochimeric antibody, a caninized antibody, a felinized antibody, a fully canine antibody, a fully feline antibody, or a fully equine antibody. The term “antibody” preferably refers to monoclonal antibodies and fragments thereof, and immunologic binding equivalents thereof that can bind to the IL-31 protein and fragments thereof. The term antibody is used both to refer to a homogeneous molecular, or a mixture such as a serum product made up of a plurality of different molecular entities.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

The term “variable” region comprises framework and CDRs (otherwise known as hypervariables) and refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework region (FR). The variable domains of native heavy and light chains each comprise multiple FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen binding. The hypervariable region comprises amino acid residues from a “complementarity determining region” or “CDR” (Kabat, et al. (1991), above) and/or those residues from a “hypervariable loop” (Chothia and Lesk J. Mol. Biol. 196:901-917 (1987). “Framework” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. Presently there are five major classes of immunoglobulins: IgA, IgD, IgE, IgG, and IgM, and several of these may be further divided into subclasses (isotypes), e.g., IgG1, IgG2, IgG3, IgG4, IgA, and IgA2 (as defined by mouse and human designation). The heavy-chain constant domains that correspond to the different classes of immunoglobulins are called alpha, delta, epsilon, gamma, and mu, respectively. The subunit structures and three-dimensional configurations of different classes of immunoglobulins are well known in multiple species. The prevalence of individual isotypes and functional activities associated with these constant domains are species-specific and must be experimentally defined.

“Monoclonal antibody” as defined herein is an antibody produced by a single clone of cells (specifically, a single clone of cells, such as hybridoma cells) and therefore a single pure homogeneous type of antibody. All monoclonal antibodies produced from the same clone are identical and have the same antigen specificity. The term “monoclonal” pertains to a single clone of cells, a single cell, and the progeny of that cell.

“Fully canine antibody” as defined herein is a monoclonal antibody produced by a clone of cells (typically a CHO cell line) and therefore a single pure homogeneous type of antibody. Antibodies identified from single B cells of immunized mammals, such as dogs are created as recombinant IgG proteins following identification of their variable domain sequences. Grafting of these variable domains onto canine constant domains (heavy chain and light chain kappa or lambda constant) results in the generation of recombinant fully canine antibodies. All fully canine monoclonal antibodies produced from the same clone are identical and have the same antigen specificity. The term “monoclonal” pertains to a single clone of cells, a single cell, and the progeny of that cell.

“Fully feline antibody” as defined herein is a monoclonal antibody produced by a clone of cells (typically a CHO cell line) and therefore a single pure homogeneous type of antibody. Antibodies identified from single B cells of immunized mammals, such as dogs are created as recombinant IgG proteins following identification of their variable domain sequences. Grafting of these variable domains onto feline constant domains (heavy chain and light chain kappa or lambda constant) results in the generation of recombinant fully feline antibodies. All fully feline monoclonal antibodies produced from the same clone are identical and have the same antigen specificity. The term “monoclonal” pertains to a single clone of cells, a single cell, and the progeny of that cell.

“Fully equine antibody” as defined herein is a monoclonal antibody produced by a clone of cells (typically a CHO cell line) and therefore a single pure homogeneous type of antibody. Antibodies identified from single B cells of immunized mammals, such as dogs are created as recombinant IgG proteins following identification of their variable domain sequences. Grafting of these variable domains onto equine constant domains (heavy chain and light chain kappa or lambda constant) results in the generation of recombinant fully equine antibodies. All fully equine monoclonal antibodies produced from the same clone are identical and have the same antigen specificity. The term “monoclonal” pertains to a single clone of cells, a single cell, and the progeny of that cell.

The monoclonal antibodies herein specifically include “chimeric” antibodies (immunoglobulins) in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity. Typically, chimeric antibodies are antibodies whose light and heavy chain genes have been constructed, typically by genetic engineering, from antibody variable and constant region genes belonging to different species. For example, the variable segments of the genes from a mouse monoclonal antibody may be joined to canine constant segments. In one embodiment of a chimeric mouse:canine IgG, the antigen binding site is derived from mouse while the F_{c} portion is canine.

“Caninized” forms of non-canine (e.g., murine) antibodies are genetically engineered antibodies that contain minimal sequence derived from non-canine immunoglobulin. Caninized antibodies are canine immunoglobulin sequences (recipient antibody) in which hypervariable region residues of the recipient are replaced by hypervariable region residues from a non-canine species (donor antibody) such as mouse having the desired specificity, affinity, and capacity. In some instances, framework region (FR) residues of the canine immunoglobulin sequences are replaced by corresponding non-canine residues. Furthermore, caninized antibodies may include residues that are not found in the recipient antibody or in the donor antibody. These modifications are made to further refine antibody performance. In general, the caninized antibody will include substantially all of at least one, and typically two, variable domains, in which all or substantially all of the hypervariable regions correspond to those of a non-canine immunoglobulin sequence and all or substantially all of the FRs are those of a canine immunoglobulin sequence. The caninized antibody optionally also will comprise a complete, or at least a portion of an immunoglobulin constant region (Fc), typically that of a canine immunoglobulin sequence. In one embodiment of speciation or caninization of a mouse IgG, mouse CDRs are grafted onto canine frameworks.

“Felinized” forms of non-feline (e.g., murine) antibodies are genetically engineered antibodies that contain minimal sequence derived from non-feline immunoglobulin. Felinized antibodies are feline immunoglobulin sequences (recipient antibody) in which hypervariable region residues of the recipient are replaced by hypervariable region residues from a non-feline species (donor antibody) such as mouse having the desired specificity, affinity, and capacity. In some instances, framework region (FR) residues of the feline immunoglobulin sequences are replaced by corresponding non-feline residues. Furthermore, felinized antibodies may include residues that are not found in the recipient antibody or in the donor antibody. These modifications are made to further refine antibody performance. In general, the felinized antibody will include substantially all of at least one, and typically two, variable domains, in which all or substantially all of the hypervariable regions correspond to those of a non-feline immunoglobulin sequence and all or substantially all of the FRs are those of a feline immunoglobulin sequence. The felinized antibody optionally also will comprise a complete, or at least a portion of an immunoglobulin constant region (Fc), typically that of a feline immunoglobulin sequence.

“Equinized” forms of non-equine (e.g., murine) antibodies are genetically engineered antibodies that contain minimal sequence derived from non-equine immunoglobulin. Equinized antibodies are equine immunoglobulin sequences (recipient antibody) in which hypervariable region residues of the recipient are replaced by hypervariable region residues from a non-equine species (donor antibody) such as mouse having the desired specificity, affinity, and capacity. In some instances, framework region (FR) residues of the equine immunoglobulin sequences are replaced by corresponding non-equine residues. Furthermore, equinized antibodies may include residues that are not found in the recipient antibody or in the donor antibody. These modifications are made to further refine antibody performance. In general, the equinized antibody will include substantially all of at least one, and typically two, variable domains, in which all or substantially all of the hypervariable regions correspond to those of a non-equine immunoglobulin sequence and all or substantially all of the FRs are those of an equine immunoglobulin sequence. The equinized antibody optionally also will comprise a complete, or at least a portion of an immunoglobulin constant region (Fc), typically that of an equine immunoglobulin sequence.

“Fully Canine” antibodies are genetically engineered antibodies that contain no sequence derived from non-canine immunoglobulin. Fully canine antibodies are canine immunoglobulin sequences (recipient antibody) in which hypervariable region residues are derived from a naturally occurring canine antibody (donor antibody) having the desired specificity, affinity, and capacity. In some instances, framework region (FR) residues of the canine immunoglobulin sequences are replaced by corresponding non-canine residues. Furthermore, fully canine antibodies may include residues that are not found in the recipient antibody or in the donor antibody, such as including, but not limited to changes in the CDRs to modify affinity. These modifications are made to further refine antibody performance. In general, the fully canine antibody will include substantially all of at least one, and typically two, variable domains, in which all or substantially all of the hypervariable regions correspond to those of a canine immunoglobulin sequence and all or substantially all of the FRs are those of an canine immunoglobulin sequence. The fully canine antibody optionally also will comprise a complete, or at least a portion of an immunoglobulin constant region (Fc), typically that of canine immunoglobulin sequence.

“Fully Feline” antibodies are genetically engineered antibodies that contain no sequence derived from non-feline immunoglobulin. Fully feline antibodies are feline immunoglobulin sequences (recipient antibody) in which hypervariable region residues are derived from a naturally occurring feline antibody (donor antibody) having the desired specificity, affinity, and capacity. In some instances, framework region (FR) residues of the feline immunoglobulin sequences are replaced by corresponding non-feline residues. Furthermore, fully feline antibodies may include residues that are not found in the recipient antibody or in the donor antibody, such as including, but not limited to changes in the CDRs to modify affinity. These modifications are made to further refine antibody performance. In general, the fully feline antibody will include substantially all of at least one, and typically two, variable domains, in which all or substantially all of the hypervariable regions correspond to those of a feline immunoglobulin sequence and all or substantially all of the FRs are those of an feline immunoglobulin sequence. The fully feline antibody optionally also will comprise a complete, or at least a portion of an immunoglobulin constant region (Fc), typically that of feline immunoglobulin sequence.

“Fully Equine” antibodies are genetically engineered antibodies that contain no sequence derived from non-equine immunoglobulin. Fully equine antibodies are equine immunoglobulin sequences (recipient antibody) in which hypervariable region residues are derived from a naturally occurring equine antibody (donor antibody) having the desired specificity, affinity, and capacity. In some instances, framework region (FR) residues of the equine immunoglobulin sequences are replaced by corresponding non-equine residues. Furthermore, fully equine antibodies may include residues that are not found in the recipient antibody or in the donor antibody, such as including, but not limited to changes in the CDRs to modify affinity. These modifications are made to further refine antibody performance. In general, the fully equine antibody will include substantially all of at least one, and typically two, variable domains, in which all or substantially all of the hypervariable regions correspond to those of a equine immunoglobulin sequence and all or substantially all of the FRs are those of an equine immunoglobulin sequence. The fully equine antibody optionally also will comprise a complete, or at least a portion of an immunoglobulin constant region (Fc), typically that of equine immunoglobulin sequence.

The term “heterochimeric” as defined herein, refers to an antibody in which one of the antibody chains (heavy or light) is caninized, felinized, or equinized while the other is chimeric. In one embodiment, a felinized variable heavy chain (where all of the CDRs are mouse and all FRs are feline) is paired with a chimeric variable light chain (where all of the CDRs are mouse and all FRs are mouse. In this embodiment, both the variable heavy and variable light chains are fused to a feline constant region.

A “variant” anti-IL-31 antibody, refers herein to a molecule which differs in amino acid sequence from a “parent” anti-IL-31 antibody amino acid sequence by virtue of addition, deletion, and/or substitution of one or more amino acid residue(s) in the parent antibody sequence and retains at least one desired activity of the parent anti-IL-31-antibody. Desired activities can include the ability to bind the antigen specifically, the ability to reduce, inhibit or neutralize IL-31 activity in an animal, and the ability to inhibit IL-31-mediated pSTAT signaling in a cell-based assay. In one embodiment, the variant comprises one or more amino acid substitution(s) in one or more hypervariable and/or framework region(s) of the parent antibody. For example, the variant may comprise at least one, e.g. from about one to about ten, and preferably from about two to about five, substitutions in one or more hypervariable and/or framework regions of the parent antibody. Ordinarily, the variant will have an amino acid sequence having at least 50% amino acid sequence identity with the parent antibody heavy or light chain variable domain sequences, more preferably at least 65%, more preferably at least 75%, more preferably at least 80%, more preferably at least 85%, more preferably at least 90%, and most preferably at least 95% sequence identity. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind an IL-31 and preferably has desired activities which are superior to those of the parent antibody. For example, the variant may have a stronger binding affinity, enhanced ability to reduce, inhibit or neutralize IL-31 activity in an animal, and/or enhanced ability to inhibit IL-31-mediated pSTAT signaling in a cell-based assay.

A “variant” nucleic acid refers herein to a molecule which differs in sequence from a “parent” nucleic acid. Polynucleotide sequence divergence may result from mutational changes such as deletions, substitutions, or additions of one or more nucleotides. Each of these changes may occur alone or in combination, one or more times in a given sequence.

The “parent” antibody herein is one that is encoded by an amino acid sequence used for the preparation of the variant. In one embodiment, the parent antibody has a canine framework region and, if present, has canine antibody constant region(s). For example, the parent antibody may be a caninized or canine antibody. As another example, the parent antibody may be a felinized or feline antibody. As yet another example, the parent antibody may be an equinized or equine antibody. In a still further example, the parent antibody is a murine monoclonal antibody.

The term “antigen binding region”, “antigen-binding portion”, and the like as used throughout the specification and claims refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. The antigen-binding portion of an antibody according to the present invention may alternatively be referred to herein as an IL-31-specific peptide or polypeptide or as an anti-IL-31 peptide or polypeptide, for example.

The term “isolated” means that the material (e.g., antibody or nucleic acid) is separated and/or recovered from a component of its natural environment. Contaminant components of its natural environment are materials that would interfere with diagnostic or therapeutic uses for the material, and may include enzymes, hormones, and other proteinaceous or nonproteinaceous solutes. With respect to nucleic acid, an isolated nucleic acid may include one that is separated from the 5′ to 3′ sequences with which it is normally associated in the chromosome. In preferred embodiments, the material will be purified to greater than 95% by weight of the material, and most preferably more than 99% by weight. Isolated material includes the material in situ within recombinant cells since at least one component of the material's natural environment will not be present. Ordinarily, however, isolated material will be prepared by at least one purification step.

The word “label” when used herein refers to a detectable compound or composition that is conjugated directly or indirectly to the antibody or nucleic acid. The label may itself be detectable by itself (e.g., radioisotope labels or fluorescent labels) or, in the case of an enzymatic label, may catalyze chemical alteration of a substrate compound or composition that is detectable.

The terms “nucleic acid”, “polynucleotide”, “nucleic acid molecule” and the like may be used interchangeably herein and refer to a series of nucleotide bases (also called “nucleotides”) in DNA and RNA. The nucleic acid may contain deoxyribonucleotides, ribonucleotides, and/or their analogs. The term “nucleic acid” includes, for example, single-stranded and double-stranded molecules. A nucleic acid can be, for example, a gene or gene fragment, exons, introns, a DNA molecule (e.g., cDNA), an RNA molecule (e.g., mRNA), recombinant nucleic acids, plasmids, and other vectors, primers and probes. Both 5′ to 3′ (sense) and 3′ to 5′ (antisense) polynucleotides are included.

A “subject” or “patient” refers to an animal in need of treatment that can be affected by molecules of the invention. Animals that can be treated in accordance with the invention include vertebrates, with mammals such as canine, feline, and equine animals being particularly preferred examples.

A “therapeutically effective amount” (or “effective amount”) refers to an amount of an active ingredient, e.g., an agent according to the invention, sufficient to effect beneficial or desired results when administered to a subject or patient. An effective amount can be administered in one or more administrations, applications or dosages. A therapeutically effective amount of a composition according to the invention may be readily determined by one of ordinary skill in the art. In the context of this invention, a “therapeutically effective amount” is one that produces an objectively measured change in one or more parameters associated with treatment of a pruritic condition or an allergic condition including clinical improvement in symptoms. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art.

As used herein, the term “therapeutic” encompasses the full spectrum of treatments for a disease or disorder. A “therapeutic” agent of the invention may act in a manner that is prophylactic or preventive, including those that incorporate procedures designed to target animals that can be identified as being at risk (pharmacogenetics); or in a manner that is ameliorative or curative in nature; or may act to slow the rate or extent of the progression of at least one symptom of a disease or disorder being treated.

“Treatment”, “treating”, and the like refers to both therapeutic treatment and prophylactic or preventative measures. Animals in need of treatment include those already with the disorder as well as those in which the disorder is to be prevented. The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “allergic condition” is defined herein as a disorder or disease caused by an interaction between the immune system and a substance foreign to the body. This foreign substance is termed “an allergen”. Common allergens include aeroallergens, such as pollens, dust, molds, dust mite proteins, injected saliva from insect bites, etc. Examples of allergic conditions include, but are not limited to, the following: allergic dermatitis, summer eczema, urticaria, heaves, inflammatory airway disease, recurrent airway obstruction, airway hyper-responsiveness, chronic obstructive pulmonary disease, and inflammatory processes resulting from autoimmunity, such as Irritable bowel syndrome (IBS).

The term “pruritic condition” is defined herein as a disease or disorder characterized by an intense itching sensation that produces the urge to rub or scratch the skin to obtain relief. Examples of pruritic conditions include, but are not limited to the following: atopic dermatitis, allergic dermatitis, eczema, psoriasis, scleroderma, and pruritus.

As used herein, the terms “cell”, “cell line”, and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell (e.g., bacterial cells, yeast cells, mammalian cells, and insect cells) whether located in vitro or in vivo. For example, host cells may be located in a transgenic animal. Host cell can be used as a recipient for vectors and may include any transformable organism that is capable of replicating a vector and/or expressing a heterologous nucleic acid encoded by a vector.

A “composition” is intended to mean a combination of active agent and another compound or composition which can be inert (e.g., a label), or active, such as an adjuvant.

As defined herein, pharmaceutically acceptable carriers suitable for use in the invention are well known to those of skill in the art. Such carriers include, without limitation, water, saline, buffered saline, phosphate buffer, alcoholic/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, histidine-HCl, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose, trehalose, sucrose, and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (e.g., EDTA), inert gases and the like may also be provided in the pharmaceutical carriers. The present invention is not limited by the selection of the carrier. The preparation of these pharmaceutically acceptable compositions, from the above-described components, having appropriate pH isotonicity, stability and other conventional characteristics is within the skill of the art. See, e.g., texts such as Remington: The Science and Practice of Pharmacy, 20th ed, Lippincott Williams & Wilkins, publ., 2000; and The Handbook of Pharmaceutical Excipients, 4.sup.th edit., eds. R. C. Rowe et al, APhA Publications, 2003.

The term “conservative amino acid substitution” indicates any amino acid substitution for a given amino acid residue, where the substitute residue is so chemically similar to that of the given residue that no substantial decrease in polypeptide function (e.g., enzymatic activity) results. Conservative amino acid substitutions are commonly known in the art and examples thereof are described, e.g., in U.S. Pat. Nos. 6,790,639, 6,774,107, 6,194,167, or 5,350,576. In a preferred embodiment, a conservative amino acid substitution will be any one that occurs within one of the following six groups
1. Small aliphatic, substantially non-polar residues: Ala, Gly, Pro, Ser, and Thr;
2. Large aliphatic, non-polar residues: Ile, Leu, and Val; Met;
3. Polar, negatively charged residues and their amides: Asp and Glu;
4. Amides of polar, negatively charged residues: Asn and Gln; His;
5. Polar, positively charged residues: Arg and Lys; His; and
6. Large aromatic residues: Trp and Tyr; Phe.
In a preferred embodiment, a conservative amino acid substitution will be any one of the following, which are listed as Native Residue (Conservative Substitutions) pairs: Ala (Ser); Arg (Lys); Asn (Gln; His); Asp (Glu); Gln (Asn); Glu (Asp); Gly (Pro); His (Asn; Gln); Ile (Leu; Val); Leu (Ile; Val); Lys (Arg; Gln; Glu); Met (Leu; Ile); Phe (Met; Leu; Tyr); Ser (Thr); Thr (Ser); Trp (Tyr); Tyr (Trp; Phe); and Val (Ile; Leu).

Just as a polypeptide may contain conservative amino acid substitution(s), a polynucleotide hereof may contain conservative codon substitution(s). A codon substitution is considered conservative if, when expressed, it produces a conservative amino acid substitution, as described above. Degenerate codon substitution, which results in no amino acid substitution, is also useful in polynucleotides according to the present invention. Thus, e.g., a polynucleotide encoding a selected polypeptide useful in an embodiment of the present invention may be mutated by degenerate codon substitution in order to approximate the codon usage frequency exhibited by an expression host cell to be transformed therewith, or to otherwise improve the expression thereof.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Unless otherwise defined, scientific and technical terms used in connection with the antibodies described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

Standard techniques are used for recombinant DNA, oligonucleotide synthesis, and tissue culture and transfection (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification, See e.g., Sambrook et al. MOLECULAR CLONING: LAB. MANUAL (3rd ed., Cold Spring Harbor Lab. Press, Cold Spring Harbor, N.Y., 2001) and Ausubel et al. Current Protocols in Molecular Biology (New York: Greene Publishing Association/Wiley Interscience), 1993. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application.

The present invention provides for recombinant monoclonal antibodies and peptides and their uses in clinical and scientific procedures, including diagnostic procedures.

With the advent of methods of molecular biology and recombinant technology, it is possible to produce antibody and antibody-like molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly-constructed, or how they are synthesized, in vitro or in vivo, using transgenic animals, large cell cultures of laboratory or commercial size, using transgenic plants, or by direct chemical synthesis employing no living organisms at any stage of the process, all antibodies have a similar overall 3 dimensional structure. This structure is often given as H₂L₂ and refers to the fact that antibodies commonly comprise two light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets.

As noted above, the term “antigen binding region”, “antigen-binding portion” and the like as used throughout the specification and claims refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. The antigen-binding portion of an antibody according to the present invention may be referred to herein as an IL-31-specific peptide or polypeptide or as an anti-IL-31 peptide or polypeptide, for example.

Within the variable regions of the H or L chains that provide for the antigen binding regions are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have three CDR regions, each non-contiguous with the others.

In all mammalian species, antibody peptides contain constant (i.e., highly conserved) and variable regions, and, within the latter, there are the CDRs and the so-called “framework regions” made up of amino acid sequences within the variable region of the heavy or light chain but outside the CDRs.

Regarding the antigenic determinate recognized by the CDR regions of the antibody, this is also referred to as the “epitope.” In other words, epitope refers to that portion of any molecule capable of being recognized by, and bound by, an antibody (the corresponding antibody binding region may be referred to as a paratope).

An “antigen” is a molecule or a portion of a molecule capable of being bound by an antibody which is additionally capable of inducing an animal to produce an antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The antibodies of the present invention are meant to include both intact immunoglobulin molecules as well as portions, fragments, peptides and derivatives thereof such as, for example, Fab, Fab′, F(ab′)₂, Fv, Fse, CDR regions, paratopes, or any portion (e.g., a polypeptide) or peptide sequence of the antibody that is capable of binding an antigen or epitope. An antibody is said to be “capable of binding” a molecule if it is capable of specifically reacting with the molecule to thereby bind the molecule to the antibody.

The antibodies of the present invention also includes chimeric antibodies, heterochimeric antibodies, caninized antibodies, felinized antibodies, or equinized antibodies, as well as fragments, portions, regions, peptides or derivatives thereof, provided by any known technique, such as, but not limited to, enzymatic cleavage, peptide synthesis, or recombinant techniques. Such antibodies of the present invention are capable of specifically binding at least one of canine IL-31 or feline IL-31. Antibody fragments or portions may lack the Fc fragment of intact antibody, clear more rapidly from the circulation, and may have less non-specific tissue binding than an intact antibody. Examples of antibody fragments may be produced from intact antibodies using methods well known in the art, for example by proteolytic cleavage with enzymes such as papain (to produce Fab fragments) or pepsin (to produce F(ab′)₂ fragments). See, e.g., Wahl et al., 24 J. Nucl. Med. 316-25 (1983). Portions of antibodies may be made by any of the above methods, or may be made by expressing a portion of the recombinant molecule. For example, the CDR region(s) of a recombinant antibody may be isolated and subcloned into the appropriate expression vector. See, e.g., U.S. Pat. No. 6,680,053.

The term “antigen binding region”, “antigen-binding portion”, and the like as used throughout the specification and claims refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. The antigen-binding portion of an antibody according to the present invention may alternatively be referred to herein as an IL-31-specific peptide or polypeptide or as an anti-IL-31 peptide or polypeptide, for example.

### Clones 15H05, ZIL1, ZIL8, ZIL9, ZIL11, ZIL69, ZIL94, ZIL154, ZIL159, ZIL171, 04H07, and 06A09 Nucleotide and Amino Acid Sequences

In some embodiments, the present invention provides for novel monoclonal antibodies that specifically bind to at least one of canine IL-31, feline IL-31, or equine IL-31. In one embodiment, a monoclonal antibody of the invention binds to canine IL-31, feline IL-31, or equine IL-31 and prevents its binding to, and activation of, its co-receptor complex comprising IL-31 receptor A (IL-31Ra) and Oncostatin-M-specific receptor (OsmR or IL-31Rb). The monoclonal antibodies of the present invention are identified herein as “15H05”, “ZIL1”, “ZIL8”, “ZIL9”, “ZIL11”, “ZIL69”, “ZIL94”, “ZIL154”, “ZIL159”, “ZIL171”, 04H07, and 06A09 which refers to the number assigned to its clone. Herein, “15H05”, “ZIL1”, “ZIL8”, “ZIL9”, “ZIL11”, “ZIL69”, “ZIL94”, “ZIL154”, “ZIL159”, “ZIL171”, “04H07”, and “06A09” also refers to the portion of the monoclonal antibody, the paratope or CDRs, that bind specifically with an IL-31 epitope identified as 15H05, ZIL1, ZIL8, ZIL9, ZIL11, ZIL69, ZIL94, ZIL154, ZIL159, ZIL171, 04H07, and 06A09 because of its ability to bind the 15H05, ZIL1, ZIL8, ZIL9, ZIL11, ZIL69, ZIL94, ZIL154, ZIL159, ZIL171, 04H07, and 06A09 antibodies, respectively. The several recombinant, chimeric, heterochimeric, caninized, felinized, equinized, fully canine, fully feline, and/or fully equine forms of 15H05, ZIL1, ZIL8, ZIL9, ZIL11, ZIL69, ZIL94, ZIL154, ZIL159, ZIL171, 04H07, and 06A09 described herein may be referred to by the same name. In some embodiments, 15H05 may be alternatively referred to herein as 1505 at least because they share the same CDRs.

In one embodiment, the present invention provides a monoclonal antibody, or antigen-binding portion thereof that specifically binds to a region on a mammalian IL-31 protein involved with interaction of the IL-31 protein with its co-receptor, wherein the binding of said antibody to said region is impacted by mutations in a 15H05 epitope binding region selected from at least one of the following: a) a region between about amino acid residues 124 and 135 of a feline IL-31 sequence represented by SEQ ID NO: 157 (Feline_IL_31 wildtype); b) a region between about amino acid residues 124 and 135 of a canine IL-31 sequence represented by SEQ ID NO: 155 (Canine_IL31); and c) a region between about amino acid residues 118 and 129 of an equine IL-31 sequence represented by SEQ ID NO: 165 (Equine_IL31). In one embodiment, the mutations in the 15H05 epitope binding region are selected from at least one of the following: (a) a mutant wherein positions 126 and 128 of SEQ ID NO: 157 are changed to Alanine; (b) a mutant wherein positions 126 and 128 of SEQ ID NO: 155 are changed to Alanine; and (c) a mutant wherein positions 120 and 122 of SEQ ID NO: 165 are changed to Alanine.

In one particular embodiment, an antibody according to the present invention binds to the 15H05 epitope binding region described above. That is to say,

in one embodiment, the present invention provides a monoclonal antibody, or antigen-binding portion thereof that specifically binds to a region on a mammalian IL-31 protein involved with interaction of the IL-31 protein with its co-receptor, wherein the binding region is selected from at least one of the following: a) a region between about amino acid residues 124 and 135 of a feline IL-31 sequence represented by SEQ ID NO: 157 (Feline_IL_31_wildtype); b) a region between about amino acid residues 124 and 135 of a canine IL-31 sequence represented by SEQ ID NO: 155 (Canine_IL31); and c) a region between about amino acid residues 118 and 129 of an equine IL-31 sequence represented by SEQ ID NO: 165 (Equine_IL31).

In one embodiment, the mammalian IL-31 to which the antibody or antigen-binding portion thereof specifically binds is feline IL-31, wherein the antibody binds to a region between about amino acid residues 125 and 134 of a feline IL-31 sequence represented by SEQ ID NO: 157 (Feline_IL_31_wildtype). In some embodiments, the antibody which binds to feline IL31 includes a VL chain comprising Framework 2 (FW2) changes selected from the following: an Asparagine in place of Lysine at position 42, an Isoleucine in place of Valine at position 43, a Valine in place of Leucine at position 46, an Asparagine in place of Lysine at position 49, and combinations thereof, wherein the positions are in reference to the numbering of SEQ ID NO: 127 (FEL_15H05_VL1).

In one embodiment, the monoclonal antibody or antigen-binding portion thereof includes the following combinations of complementary determining region (CDR) sequences:
1) antibody 15H05: variable heavy (VH)-CDR1 of SYTIH (SEQ ID NO: 1), VH-CDR2 of NINPTSGYTENNQRFKD (SEQ ID NO: 2), VH-CDR3 of WGFKYDGEWSFDV (SEQ ID NO: 3), variable light (VL)-CDR1 of RASQGISIWLS (SEQ ID NO: 4), VL-CDR2 of KASNLHI (SEQ ID NO: 5), and VL-CDR3 of LQSQTYPLT (SEQ ID NO: 6); or
2) a variant of 1) that differs from the parent antibody 15H05 by addition, deletion, and/or substitution of one or more amino acid residues in at least one of VH or VL CDR1, CDR2, or CDR3.

In one embodiment, antibody 15H05 above includes at least one of the following variable heavy and/or variable light chains:

a) a variable light chain comprising
FEL_15H05_VL1_FW2:
(SEQ ID NO: 135)
EIQMTQSPSSLSASPGDRVTITCRASQGISIWLSWYQQKPGNIPKVLINK
ASNLHIGVPSRFSGSGSGTDFTLTISSLEPEDAATYYCLQSQTYPLTFGG
GTKLEIK,
and
b) a variable heavy chain comprising
FEL_15H05_VH1:
(SEQ ID NO: 121)
QVLLVQSGAEVRTPGASVKIFCKASGYSFTSYTIHWLRQAPAQGLEWMGN
INPTSGYTENNQRFKDRLTLTADTSTNTAYMELSSLRSADTAMYYCARWG
FKYDGEWSFDVWGAGTTVTVSS.

In another embodiment, the monoclonal antibody or antigen-binding portion thereof includes the following combinations of complementary determining region (CDR) sequences:

1) antibody ZIL1:
variable heavy
(VH)-CDR1 of
(SEQ ID NO: 13)
SYGMS,
VH-CDR2 of
(SEQ ID NO: 14)
HINSGGSSTYYADAVKG,
VH-CDR3 of
(SEQ ID NO: 15)
VYTTLAAFWTDNFDY,
variable light
(VL)-CDR1 of
(SEQ ID NO: 16)
SGSTNNIGILAAT,
VL-CDR2 of
(SEQ ID NO: 17
SDGNRPS,
and
VL-CDR3 of
(SEQ ID NO: 18)
QSFDTTLDAYV;
2) antibody ZIL8:
VH-CDR1 of
(SEQ ID NO: 19)
DYAMS,
VH-CDR2 of
(SEQ ID NO: 20)
GIDSVGSGTSYADAVKG,
VH-CDR3 of
(SEQ ID NO: 21)
GFPGSFEH,
VL-CDR1 of
(SEQ ID NO: 22)
TGSSSNIGSGYVG,
VL-CDR2 of
(SEQ ID NO: 23)
YNSDRPS,
VL-CDR3 of
(SEQ ID NO: 24)
SVYDRTFNAV;
3) antibody ZIL9:
VH-CDR1 of
(SEQ ID NO: 25)
SYDMT,
VH-CDR2 of
(SEQ ID NO: 26)
DVNSGGTGTAYAVAVKG,
VH-CDR3 of
(SEQ ID NO: 27)
LGVRDGLSV,
VL-CDR1 of
(SEQ ID NO: 28)
SGESLNEYYTQ,
VL-CDR2 of
(SEQ ID NO: 29)
RDTERPS,
VL-CDR3 of
(SEQ ID NO: 30)
ESAVDTGTLV;
4) antibody ZIL11:
VH-CDR1 of
(SEQ ID NO: 31)
TYVMN,
VH-CDR2 of
(SEQ ID NO: 32)
SINGGGSSPTYADAVRG,
VH-CDR3 of
(SEQ ID NO: 33)
SMVGPFDY,
VL-CDR1 of
(SEQ ID NO: 34)
SGESLSNYYAQ,
VL-CDR2 of
(SEQ ID NO: 35)
KDTERPS,
VL-CDR3 of
(SEQ ID NO: 36)
ESAVSSDTIV;
5) antibody ZIL69:
VH-CDR1 of
(SEQ ID NO: 37)
SYAMK,
VH-CDR2 of
(SEQ ID NO: 38)
TINNDGTRTGYADAVRG,
VH-CDR3 of
(SEQ ID NO: 39)
GNAESGCTGDHCPPY,
VL-CDR1 of
(SEQ ID NO: 40)
SGESLNKYYAQ,
VL-CDR2 of
(SEQ ID NO: 41)
KDTERPS,
VL-CDR3 of
(SEQ ID NO: 42)
ESAVSSETNV;
6) antibody ZIL94:
VH-CDR1 of
(SEQ ID NO: 43)
TYFMS,
VH-CDR2 of
(SEQ ID NO: 44)
LISSDGSGTYYADAVKG,
VH-CDR3 of
(SEQ ID NO: 45)
FWRAFND,
VL-CDR1 of
(SEQ ID NO: 46)
GLNSGSVSTSNYPG,
VL-CDR2 of
(SEQ ID NO: 47)
DTGSRPS,
VL-CDR3 of
(SEQ ID NO: 48)
SLYTDSDILV;
7) antibody ZIL154:
VH-CDR1 of
(SEQ ID NO: 49)
DRGMS,
VH-CDR2 of
(SEQ ID NO: 50)
YIRYDGSRTDYADAVEG,
VH-CDR3 of
(SEQ ID NO: 51)
WDGSSFDY,
VL-CDR1 of
(SEQ ID NO: 52)
KASQSLLHSDGNTYLD,
VL-CDR2 of
(SEQ ID NO: 53)
KVSNRDP,
VL-CDR3 of
(SEQ ID NO: 54)
MQAIHFPLT;
8) antibody ZIL159:
VH-CDR1 of
(SEQ ID NO: 55)
SYVMT,
VH-CDR2 of
(SEQ ID NO: 56)
GINSEGSRTAYADAVKG,
VH-CDR3 of
(SEQ ID NO: 57)
GDIVATGTSY,
VL-CDR1 of
(SEQ ID NO: 58)
SGETLNRFYTQ,
VL-CDR2 of
(SEQ ID NO: 59)
KDTERPS,
VL-CDR3 of
(SEQ ID NO: 60)
KSAVSIDVGV;
9) antibody ZIL171:
VH-CDR1 of
(SEQ ID NO: 61)
TYVMN,
VH-CDR2 of
(SEQ ID NO: 62)
SINGGGSSPTYADAVRG,
VH-CDR3 of
(SEQ ID NO: 63)
SMVGPFDY,
VL-CDR1 of
(SEQ ID NO: 64)
SGKSLSYYYAQ,
VL-CDR2 of
(SEQ ID NO: 65)
KDTERPS,
VL-CDR3 of
(SEQ ID NO: 66)
ESAVSSDTIV;
10) antibody 04H07:
VH-CDR1 of
(SEQ ID NO: 200)
SYWMN,
VH-CDR2 of
(SEQ ID NO: 201)
MIDPSDSEIHYNQVFKD,
VH-CDR3 of
(SEQ ID NO: 202)
QDIVTTVDY,
VL-CDR1 of
(SEQ ID NO: 203)
KSSQSLLYSINQKNHLA,
VL-CDR2 of
(SEQ ID NO: 204)
WASTRES,
VL-CDR3 of
(SEQ ID NO: 205)
QQGYTYPFT;
11) antibody 06A09:
VH-CDR1 of
(SEQ ID NO: 206)
SYWMN,
VH-CDR2 of
(SEQ ID NO: 207)
MIDPSDSETHYNQIFRD,
VH-CDR3 of
(SEQ ID NO: 208)
QDIVTTVDY,
VL-CDR1 of
(SEQ ID NO: 209)
KSSQSLLYSINQKNFLA,
VL-CDR2 of
(SEQ ID NO: 210)
WASTRES,
VL-CDR3 of
(SEQ ID NO: 211)
QQHYGYPFT;
or
12) a variant of 1) to 11) that differs from
respective parent antibody ZIL1, ZIL8, ZIL9,
ZIL11, ZIL69, ZIL94, ZIL154, ZIL159, ZIL171,
04H07, or 06A09 by addition, deletion, and/or
substitution of one or more amino acid residues
in at least oneof VH or VL CDR1, CDR2, or CDR3.

In some embodiments of the present invention,
1) antibody ZIL1 includes at least one of the following:

a) a variable light chain comprising
CAN-ZIL1_VL:
(SEQ ID NO: 77)
QSVLTQPTSVSGSLGQRVTISCSGSTNNIGILAATWYQQLPGKAPKVLVY
SDGNRPSGVPDRFSGSKSGNSATLTITGLQAEDEADYYCQSFDTTLDAYV
FGSGTQLTVL,
and
b) a variable heavy chain comprising
CAN-ZIL1_VH:
(SEQ ID NO: 75)
EVQLVESGGDLVKPGGSLRLSCVASGFTFSSYGMSWVRQAPGKGLQWVAH
INSGGSSTYYADAVKGRFTISRDNAKNTLYLQMNSLRAEDTAVYYCVEVY
TTLAAFWTDNFDYWGQGTLVTVSS;

2) antibody ZIL8 includes at least one of the following:

a) a variable light chain comprising CAN-ZIL8_VL:
(SEQ ID NO: 81)
QSVLTQPASVSGSLGQKVTISCTGSSSNIGSGYVGWYQQLPGTGPRTLI
YYNSDRPSGVPDRFSGSRSGTTATLTISGLQAEDEADYYCSVYDRTFNA
VFGGGT,
and
b) a variable heavy chain comprising CAN-ZIL8_VH:
(SEQ ID NO: 79)
EVQLVESGGDLVKPAGSLRLSCVASGFTFSDYAMSWVRQAPGRGLOWVA
GIDSVGSGTSYADAVKGRFTISRDDAKNTLYLQMFNLRAEDTAIYYCAS
GFPGSFEHWGQGTLVTVSS;
or
antibody ZIL8 includes at least one of the
following:
c) a variable light chain comprising ZTS_5864_VL:
(SEQ ID NO: 230)
QSVLTQPSSVSGTLGQRITISCTGSSSNIGSGYVGWYQQVPGMGPKTVI
YYNSDRPSGVPDRFSGSKSGSSGTLTITGLQAEDEADYYCSVYDRTFNA
VFGGGTHLTVLGQPKSAPPRSHSSRPISYAVFCL,
and
d) a variable heavy chain comprising ZTS_5864_VH:
(SEQ ID NO: 228)
DVQLVESGGDLVKPGGSLRLTCVASGFTFSDYAMSWVRQAPGKGLQWVA
GIDSVGSGTSYADSVKGRFTISRDNAKNTLYLQMNSLKTEDTATYYCAS
GFPGSFEHWGQGALVTVSS;
or
antibody ZIL8 includes at least one of the
following:
e) a variable light chain comprising ZTS_5865_VL:
(SEQ ID NO: 234)
SVLTQPSSVSGTLGQRITISCTGSSSNIGSGYVGWYQQVPGMGPKTVIY
YNSDRPSGVPDRFSGSKSGSSGTLTITGLQAEDEADYYCSVYDRTFNAV
FGGGTHLTVLGQPKSAPPRSHSSRPISYAVFCL,
and
f) a variable heavy chain comprising ZTS_5865_VH:
(SEQ ID NO: 232)
DVQLVESGGDLVKPGGSLRLTCVASGFTFSDYAMNWVRQAPGKGLQWVA
GIDSVGSGTSYADSVKGRFTISRDNAKNTLYLQMSGLKTEDTATYYCAS
GFPGSFEHWGQGTLVTVSS;

3) antibody ZIL9 includes at least one of the following:

a) a variable light chain comprising CAN-ZIL9_VL:
(SEQ ID NO: 85)
SSVLTQPPSVSVSLGQTATISCSGESLNEYYTQWFQQKAGQAPVLVIYR
DTERPSGIPDRFSGSSSGNTHTLTISGARAEDEADYYCESAVDTGTLVF
GGGTHLAVL,
and
b) a variable heavy chain comprising CAN-ZIL9_VH:
(SEQ ID NO: 83)
EVQLVESGGDLVKPPGSLRLSCVASGFTFSSYDMTWVRQAPGKGLQWVA
DVNSGGTGTAYAVAVKGRFTISRDNAKKTLYLQMNSLRAEDTAVYYCAK
LGVRDGLSVWGQGTLVTVSS;

4) antibody ZIL11 includes at least one of the following:

a) a variable light chain comprising CAN-ZIL11_VL:
(SEQ ID NO: 89)
SSVLTQPPSVSVSLGQTATISCSGESLSNYYAQWFQQKAGQAPVLVIYK
DTERPSGIPDRFSGSSSGNTHTLTISGARAEDEADYYCESAVSSDTIVF
GGGT,
and
b) a variable heavy chain comprising CAN-ZIL11_VH:
(SEQ ID NO: 87)
EVQLVESGGDLVKPAGSLRLSCVASGFTFRTYVMNWVRQAPGKGLQWVA
SINGGGSSPTYADAVRGRFTVSRDNAQNSLFLQMNSLRAEDTAVYFCVV
SMVGPFDYWGQGTLVTVSS;

5) antibody ZIL69 includes at least one of the following:

a) a variable light chain comprising CAN-ZIL69_VL:
(SEQ ID NO: 93)
SSVLTQPPSVSVSLGQTATISCSGESLNKYYAQWFQQKAGQAPVLVIYK
DTERPSGIPDRFSGSSAGNTHTLTISGARAEDEADYYCESAVSSETNVF
GSGTQLTVL,
and
b) a variable heavy chain comprising CAN-ZIL69_VH:
(SEQ ID NO: 91)
EVQLVESGGDLVKPAGSLRLSCVASGFTFSSYAMKWVRQAPGKGLQWVA
TINNDGTRTGYADAVRGRFTISKDNAKNTLYLQMDSLRADDTAVYYCTK
GNAESGCTGDHCPPYWGQGTLVTVSS;

6) antibody ZIL94 includes at least one of the following:

a) a variable light chain comprising CAN-ZIL94_VL:
(SEQ ID NO: 97)
QTVVIQEPSLSVSPGGTVTLTCGLNSGSVSTSNYPGWYQQTRGRTPRTI
IYDTGSRPSGVPNRFSGSISGNKAALTITGAQPEDEADYYCSLYTDSDI
LVFGGGTHLTVL,
and
b) a variable heavy chain comprising CAN-ZIL94_VH:
(SEQ ID NO: 95)
EVQLVDSGGDLVKPGGSLRLSCVASGFTFSTYFMSWVRQAPGRGLQWVA
LISSDGSGTYYADAVKGRFTISRDNAKNTLYLQMNSLRAEDTAMYYCAI
FWRAFNDWGQGTLVTVSS;

7) antibody ZIL154 includes at least one of the following:

a) a variable light chain comprising CAN-
ZIL154_VL:
(SEQ ID NO: 101)
DIVVTQTPLSLSVSPGETASFSCKASQSLLHSDGNTYLDWFRQKPGQSP
QRLIYKVSNRDPGVPDRFSGSGSGTDFTLRISGVEADDAGLYYCMQAIH
FPLTFGAGTKVELK,
and
b) a variable heavy chain comprising CAN-
ZIL154_VH:
(SEQ ID NO: 99)
EVHLVESGGDLVKPWGSLRLSCVASGFTFSDRGMSWVRQSPGKGLQWVA
YIRYDGSRTDYADAVEGRFTISRDNAKNTLYLQMNSLRAEDTAVYYCAR
WDGSSFDYWGQGTLVTVSS;

8) antibody ZIL159 includes at least one of the following:

a) a variable light chain comprising CAN-
ZIL159_VL:
(SEQ ID NO: 105)
SNVLTQPPSVSVSLGQTATISCSGETLNRFYTQWFQQKAGQAPVLVIYK
DTERPSGIPDRFSGSSSGNIHTLTISGARAEDEAAYYCKSAVSIDVGVF
GGGTHLTVF,
and
b) a variable heavy chain comprising CAN-
ZIL159_VH:
(SEQ ID NO: 103)
EVQLVESGGDLVKPAGSLRLSCVASGFTFSSYVMTWVRQAPGKGLQWVA
GINSEGSRTAYADAVKGRFTISRDNAKNTLYLQIDSLRAEDTAIYYCAT
GDIVATGTSYWGQGTLVTVSS;

9) antibody ZIL171 includes at least one of the following:

a) a variable light chain comprising CAN-
ZIL171_VL:
(SEQ ID NO: 109)
SSVLTQPPSVSVSLGQTATISCSGKSLSYYYAQWFQQKAGQAPVLVIYK
DTERPSGIPDRFSGSSSGNTHTLTISGARAEDEADYYCESAVSSDTIVF
GGGTHLTVL,
and
b) a variable heavy chain comprising CAN-
ZIL171_VH:
(SEQ ID NO: 107)
EVQLVESGGDLVKPAGSLRLSCVASGFTFRTYVMNWVRQAPGKGLQWVA
SINGGGSSPTYADAVRGRFTVSRDNAQNSLFLQMNSLRAEDTAIYFCVV
SMVGPFDYWGHGTLVTVSS;
and

10) antibody 04H07 includes at least one of the following:

a) a variable light chain comprising Mu_04H07_VL:
(SEQ ID NO: 214)
DIVMSQSPSSLAVSVGEKVTMSCKSSQSLLYSINQKNHLAWFQQKPGQS
PKLLIYWASTRESGVPARFTGSGSGTDFTLTISSVKTEDLAVYYCQQGY
TYPFTFGSGTKLEIK,
and
b) a variable heavy chain comprising Mu_04H07_VH:
(SEQ ID NO: 212)
QVQLQQPGAELVRPGASVKLSCKASGYTFTSYWMNWAKQRPGQGLEWIG
MIDPSDSEIHYNQVFKDKATLTVDKSSSTAYMQLSSLTSEDSAVYYCAR
QDIVTTVDYWGQGTTLTVSS;
and

11) antibody 06A09 includes at least one of the following:

a) a variable light chain comprising Mu_06A09_VL:
(SEQ ID NO: 218)
DIVMSQSPSSLAVSVGEKVTMSCKSSQSLLYSINQKNFLAWYQQKPGQS
PKLLIYWASTRESGVPDRFTGSGSGTDFTLTISSVKSEDLAVYYCQQHY
GYPFTFGSGTKLEIK,
and
b) a variable heavy chain comprising Mu_06A09_VH:
(SEQ ID NO: 216)
QVQLQQPGAELVRPGASVKLSCKAYGYTFTSYWMNWVKQRPGQGLEWIG
MIDPSDSETHYNQIFRDKATLTIDKSSSTAYMQLSSLTSEDSAVYFCAR
QDIVTTVDYWGQGTTLTVSS.

In other embodiments, the invention provides a host cell that produces an antibody described above.

The present invention also includes, within its scope, nucleotide sequences encoding the variable regions of the light and heavy chains of the anti-IL-31 antibody of the present invention. Included also within the scope of the invention is any nucleotide sequence that encodes the amino acid sequence of 15H05, ZIL1, ZIL8, ZIL9, ZIL11, ZIL69, ZIL94, ZIL154, ZIL159, ZIL171, 04H07, 06A09, or IL-31-specific polypeptides or peptides thereof.

In some embodiments, the invention provides an isolated nucleic acid including a nucleic acid sequence encoding at least one of the following combinations of variable heavy complementary determining region (CDR) sequences:

1)
15H05: variable heavy
(VH)-CDR1 of
(SEQ ID NO: 1)
SYTIH,
VH-CDR2 of
(SEQ ID NO: 2)
NINPTSGYTENNQRFKD,
and
VH-CDR3 of
(SEQ ID NO: 3)
WGFKYDGEWSFDV;
2) ZIL1:
VH-CDR1 of
(SEQ ID NO: 13)
SYGMS,
VH-CDR2 of
(SEQ ID NO: 14)
HINSGGSSTYYADAVKG,
and
VH-CDR3 of
(SEQ ID NO: 15)
VYTTLAAFWTDNFDY;
3) ZIL8:
VH-CDR1 of
(SEQ ID NO: 19)
DYAMS,
VH-CDR2 of
(SEQ ID NO: 20)
GIDSVGSGTSYADAVKG,
and
VH-CDR3 of
(SEQ ID NO: 21)
GFPGSFEH;
4) ZIL9:
VH-CDR1 of
(SEQ ID NO: 25)
SYDMT,
VH-CDR2 of
(SEQ ID NO: 26)
DVNSGGTGTAYAVAVKG,
and
VH-CDR3 of
(SEQ ID NO: 27)
LGVRDGLSV;
5) ZIL11:
VH-CDR1 of
(SEQ ID NO: 31)
TYVMN,
VH-CDR2 of
(SEQ ID NO: 32)
SINGGGSSPTYADAVRG,
and
VH-CDR3 of
(SEQ ID NO: 33)
SMVGPFDY;
6) ZIL69:
VH-CDR1 of
(SEQ ID NO: 37)
SYAMK,
VH-CDR2 of
(SEQ ID NO: 38)
TINNDGTRTGYADAVRG,
and
VH-CDR3 of
(SEQ ID NO: 39)
GNAESGCTGDHCPPY;
7) ZIL94:
VH-CDR1 of
(SEQ ID NO: 43)
TYFMS,
VH-CDR2 of
(SEQ ID NO: 44)
LISSDGSGTYYADAVKG,
and
VH-CDR3 of
(SEQ ID NO: 45)
FWRAFND
8) ZIL154:
VH-CDR1 of
(SEQ ID NO: 49)
DRGMS,
VH-CDR2 of
(SEQ ID NO: 50)
YIRYDGSRTDYADAVEG,
and
VH-CDR3 of
(SEQ ID NO: 51)
WDGSSFDY;
9) ZIL159:
VH-CDR1 of
(SEQ ID NO: 55)
SYVMT,
VH-CDR2 of
(SEQ ID NO: 56)
GINSEGSRTAYADAVKG,
and
VH-CDR3 of
(SEQ ID NO: 57)
GDIVATGTSY;
10) ZIL171:
VH-CDR1 of
(SEQ ID NO: 61)
TYVMN,
VH-CDR2 of
(SEQ ID NO: 62)
SINGGGSSPTYADAVRG,
and
VH-CDR3 of
(SEQ ID NO: 63)
SMVGPFDY,
11) 04H07:
VH-CDR1 of
(SEQ ID NO: 200)
SYWMN,
VH-CDR2 of
(SEQ ID NO: 201)
MIDPSDSEIHYNQVFKD,
and
VH-CDR3 of
(SEQ ID NO: 202)
QDIVTTVDY;
12) 06A09:
VH-CDR1 of
(SEQ ID NO: 206)
SYWMN,
VH-CDR2 of
(SEQ ID NO: 207)
MIDPSDSETHYNQIFRD,
and
VH-CDR3 of
(SEQ ID NO: 208)
QDIVTTVDY;
or
13) a variant of 1) to 12) that differs from the
CDRs of respective parent antibody 15H05, ZIL1,
ZIL8, ZIL9, ZIL11, ZIL69, ZIL94, ZIL154, ZIL159,
ZIL171, 04H07, or 06A09 by addition, deletion,
and/or substitution of one or more amino acid
residues in at least one of VH CDR1, CDR2, or
CDR3.

In one embodiment, the isolated nucleic acid described above may further include a nucleic acid sequence encoding at least one of the following combinations of variable light complementary determining region (CDR) sequences:

1) 15H05: variable light
(VL)-CDR1 of
(SEQ ID NO: 4)
RASQGISIWLS,
VL-CDR2 of
(SEQ ID NO: 5)
KASNLHI,
and
VL-CDR3 of
(SEQ ID NO: 6)
LQSQTYPLT;
2) ZIL1:
VL-CDR1 of
(SEQ ID NO: 16)
SGSTNNIGILAAT,
VL-CDR2 of
(SEQ ID NO: 17
SDGNRPS,
and
VL-CDR3 of
(SEQ ID NO: 18)
QSFDTTLDAYV;
3) ZIL8:
VL-CDR1 of
(SEQ ID NO: 22)
TGSSSNIGSGYVG,
VL-CDR2 of
(SEQ ID NO: 23)
YNSDRPS,
and
VL-CDR3 of
(SEQ ID NO: 24)
SVYDRTFNAV;
4) ZIL9:
VL-CDR1 of
(SEQ ID NO: 28)
SGESLNEYYTQ,
VL-CDR2 of
(SEQ ID NO: 29)
RDTERPS,
and
VL-CDR3 of
(SEQ ID NO: 30)
ESAVDTGTLV;
5) ZIL11:
VL-CDR1 of
(SEQ ID NO: 34)
SGESLSNYYAQ,
VL-CDR2 of
(SEQ ID NO: 35)
KDTERPS,
and
VL-CDR3 of
(SEQ ID NO: 36)
ESAVSSDTIV;
6) ZIL69:
VL-CDR1 of
(SEQ ID NO: 40)
SGESLNKYYAQ,
VL-CDR2 of
(SEQ ID NO: 41)
KDTERPS,
and
VL-CDR3 of
(SEQ ID NO: 42)
ESAVSSETNV;
7) ZIL94:
VL-CDR1 of
(SEQ ID NO: 46)
GLNSGSVSTSNYPG,
VL-CDR2 of
(SEQ ID NO: 47)
DTGSRPS,
and
VL-CDR3 of
(SEQ ID NO: 48)
SLYTDSDILV;
8) ZIL154:
VL-CDR1 of
(SEQ ID NO: 52)
KASQSLLHSDGNTYLD,
VL-CDR2 of
(SEQ ID NO: 53)
KVSNRDP,
and
VL-CDR3 of
(SEQ ID NO: 54)
MQAIHFPLT;
9) ZIL159:
VL-CDR1 of
(SEQ ID NO: 58)
SGETLNRFYTQ,
VL-CDR2 of
(SEQ ID NO: 59)
KDTERPS,
and
VL-CDR3 of
(SEQ ID NO: 60)
KSAVSIDVGV;
10) ZIL171:
VL-CDR1 of
(SEQ ID NO: 64)
SGKSLSYYYAQ,
VL-CDR2 of
(SEQ ID NO: 65)
KDTERPS,
and
VL-CDR3 of
(SEQ ID NO: 66)
ESAVSSDTIV;
11) 04H07:
VL-CDR1 of
(SEQ ID NO: 203)
KSSQSLLYSINQKNHLA,
VL-CDR2 of
(SEQ ID NO: 204)
WASTRES,
VL-CDR3 of
(SEQ ID NO: 205)
QQGYTYPFT;
12) 06A09:
VL-CDR1 of
(SEQ ID NO: 209)
KSSQSLLYSINQKNFLA,
VL-CDR2 of
(SEQ ID NO: 210)
WASTRES,
VL-CDR3 of
(SEQ ID NO: 211)
QQHYGYPFT;
or
13) a variant of 1) to 12) that differs from the
CDRs of respective parent antibody 15H05, ZIL1,
ZIL8, ZIL9, ZIL11, ZIL69, ZIL94, ZIL154, ZIL159,
ZIL171, 04H07, or 06A09 by addition, deletion,
and/or substitution of one or more amino acid
residues in at least one of VH or VL CDR1, CDR2,
or CDR3.

In one embodiment, the invention provides an isolated nucleic acid comprising a nucleic acid sequence encoding at least one of the following combinations of variable light complementary determining region (CDR) sequences:

1) 15H05: variable light
(VL)-CDR1 of
(SEQ ID NO: 4)
RASQGISIWLS,
VL-CDR2 of
(SEQ ID NO: 5)
KASNLHI,
and
VL-CDR3 of
(SEQ ID NO: 6)
LQSQTYPLT;
2) ZIL1:
VL-CDR1 of
(SEQ ID NO: 16)
SGSTNNIGILAAT,
VL-CDR2 of
(SEQ ID NO: 17
SDGNRPS,
and
VL-CDR3 of
(SEQ ID NO: 18)
QSFDTTLDAYV;
3) ZIL8:
VL-CDR1 of
(SEQ ID NO: 22)
TGSSSNIGSGYVG,
VL-CDR2 of
(SEQ ID NO: 23)
YNSDRPS,
and
VL-CDR3 of
(SEQ ID NO: 24)
SVYDRTFNAV;
4) ZIL9:
VL-CDR1 of
(SEQ ID NO: 28)
SGESLNEYYTQ,
VL-CDR2 of
(SEQ ID NO: 29)
RDTERPS,
and
VL-CDR3 of
(SEQ ID NO: 30)
ESAVDTGTLV;
5) ZIL11:
VL-CDR1 of
(SEQ ID NO: 34)
SGESLSNYYAQ,
VL-CDR2 of
(SEQ ID NO: 35)
KDTERPS,
and
VL-CDR3 of
(SEQ ID NO: 36)
ESAVSSDTIV;
6) ZIL69:
VL-CDR1 of
(SEQ ID NO: 40)
SGESLNKYYAQ,
VL-CDR2 of
(SEQ ID NO: 41)
KDTERPS,
and
VL-CDR3 of
(SEQ ID NO: 42)
ESAVSSETNV;
7) ZIL94:
VL-CDR1 of
(SEQ ID NO: 46)
GLNSGSVSTSNYPG,
VL-CDR2 of
(SEQ ID NO: 47)
DTGSRPS,
and
VL-CDR3 of
(SEQ ID NO: 48)
SLYTDSDILV;
8) ZIL154:
VL-CDR1 of
(SEQ ID NO: 52)
KASQSLLHSDGNTYLD,
VL-CDR2 of
(SEQ ID NO: 53)
KVSNRDP,
and
VL-CDR3 of
(SEQ ID NO: 54)
MQAIHFPLT;
9) ZIL159:
VL-CDR1 of
(SEQ ID NO: 58)
SGETLNRFYTQ,
VL-CDR2 of
(SEQ ID NO: 59)
KDTERPS,
and
VL-CDR3 of
(SEQ ID NO: 60)
KSAVSIDVGV;
10) ZIL171:
VL-CDR1 of
(SEQ ID NO: 64)
SGKSLSYYYAQ,
VL-CDR2 of
(SEQ ID NO: 65)
KDTERPS,
and
VL-CDR3 of
(SEQ ID NO: 66)
ESAVSSDTIV;
11) 04H07:
VL-CDR1 of
(SEQ ID NO: 203)
KSSQSLLYSINQKNHLA,
VL-CDR2 of
(SEQ ID NO: 204)
WASTRES,
VL-CDR3 of
(SEQ ID NO: 205)
QQGYTYPFT;
12) 06A09:
VL-CDR1 of
(SEQ ID NO: 209)
KSSQSLLYSINQKNFLA,
VL-CDR2 of
(SEQ ID NO: 210)
WASTRES,
VL-CDR3 of
(SEQ ID NO: 211)
QQHYGYPFT;
or
13) a variant of 1) to 12) that differs from the
CDRs of respective parent antibody 15H05, ZIL1,
ZIL8, ZIL9, ZIL11, ZIL69, ZIL94, ZIL154, ZIL159,
ZIL171, 04H07, or 06A09 by addition, deletion,
and/or substitution of one or more amino acid
residues in at least one of VH or VL CDR1, CDR2,
or CDR3.

The present invention further provides a vector including at least one of the nucleic acids described above.

As will be described in further detail below, the nucleic acid sequence encoding at least one of the above-described combinations of variable heavy complementary determining region (CDR) sequences may be contained on the same vector together with the nucleic acid sequence encoding at least one of the above-described combinations of variable light CDR sequences. Alternatively, the nucleic acid sequence encoding at least one of the above-described combinations of variable light CDR sequences and the nucleic acid sequence encoding at least one of the above-described combinations of variable heavy CDR sequences may each be contained on separate vectors.

Because the genetic code is degenerate, more than one codon can be used to encode a particular amino acid. Using the genetic code, one or more different nucleotide sequences can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an anti-IL-31 antibody or IL-31-specific portion thereof. Such “codon usage rules” are disclosed by Lathe, et al., 183 J. Molec. Biol. 1-12 (1985). Using the “codon usage rules” of Lathe, a single nucleotide sequence, or a set of nucleotide sequences that contains a theoretical “most probable” nucleotide sequence capable of encoding anti-IL-31 sequences can be identified. It is also intended that the antibody coding regions for use in the present invention could also be provided by altering existing antibody genes using standard molecular biological techniques that result in variants (agonists) of the antibodies and peptides described herein. Such variants include, but are not limited to deletions, additions and substitutions in the amino acid sequence of the anti-IL-31 antibodies or IL-31-specific polypeptides or peptides, including in the CDR regions of the antibody. For example, residues which are found to be non-critical for antigen binding within the CDR regions or other regions of the antibody can be substituted. Examples of the types of experimentation used to assess whether particular residues are non-critical for antigen binding are described in section 1.21 of the example section below. In one embodiment, one or more of the substitutions are conservative amino acid substitutions, which are described in further detail herein. However, antibody variants according to the present invention, including CDR variants are not limited to conservative amino acid substitutions.

For example, one class of substitutions is conservative amino acid substitutions. Such substitutions are those that substitute a given amino acid in an anti-IL-31 antibody peptide by another amino acid of like characteristics. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, replacements among the aromatic residues Phe, Tyr, and the like. Guidance concerning which amino acid changes are likely to be phenotypically silent is found in Bowie et al., 247 Science 1306-10 (1990).

Variant or agonist anti-IL-31 antibodies or IL-31-specific polypeptides, or peptides may be fully functional or may lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis. Cunningham et al., 244 Science 1081-85 (1989). The latter procedure introduces single alanine mutations at every residue in the molecule. The resulting mutant molecules are then tested for biological activity such as epitope binding or in vitro ADCC activity. Sites that are critical for ligand-receptor binding can also be determined by structural analysis such as crystallography, nuclear magnetic resonance, or photoaffinity labeling. Smith et al., 224 J. Mol. Biol. 899-904 (1992); de Vos et al., 255 Science 306-12 (1992).

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties (2nd ed., T. E. Creighton, W.H. Freeman & Co., NY, 1993). Many detailed reviews are available on this subject, such as by Wold, Posttranslational Covalent Modification of proteins, 1-12 (Johnson, ed., Academic Press, NY, 1983); Seifter et al. 182 Meth. Enzymol. 626-46 (1990); and Rattan et al. 663 Ann. NY Acad. Sci. 48-62 (1992).

Accordingly, the IL-31-specific antibodies, polypeptides, and peptides of the present invention also encompass derivatives or analogs in which a substituted amino acid residue is not one encoded by the genetic code.

Similarly, the additions and substitutions in the amino acid sequence as well as variations, and modifications just described may be equally applicable to the amino acid sequence of the IL-31 antigen and/or epitope or peptides thereof, and are thus encompassed by the present invention. As mentioned above, the genes encoding a monoclonal antibody according to the present invention is specifically effective in the recognition of IL-31.

### Antibody Derivatives

Included within the scope of this invention are antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

Derivatives also include radioactively labeled monoclonal antibodies that are labeled. For example, with radioactive iodine (¹²⁵I, ¹³¹I), carbon (¹⁴C), sulfur (³⁵S), indium (¹¹¹In), tritium (³H) or the like; conjugates of monoclonal antibodies with biotin or avidin, with enzymes, such as horseradish peroxidase, alkaline phosphatase, beta-D-galactosidase, glucose oxidase, glucoamylase, carboxylic acid anhydrase, acetylcholine esterase, lysozyme, malate dehydrogenase or glucose 6-phosphate dehydrogenase; and also conjugates of monoclonal antibodies with bioluminescent agents (such as luciferase), chemoluminescent agents (such as acridine esters) or fluorescent agents (such as phycobiliproteins).

Another derivative bifunctional antibody of the present invention is a bispecific antibody, generated by combining parts of two separate antibodies that recognize two different antigenic groups. This may be achieved by crosslinking or recombinant techniques. Additionally, moieties may be added to the antibody or a portion thereof to increase half-life in vivo (e.g., by lengthening the time to clearance from the blood stream. Such techniques include, for example, adding PEG moieties (also termed PEGylation), and are well-known in the art. See U.S. Patent. Appl. Pub. No. 20030031671.

### Recombinant Expression of Antibodies

In some embodiments, the nucleic acids encoding a subject monoclonal antibody are introduced directly into a host cell, and the cell is incubated under conditions sufficient to induce expression of the encoded antibody. After the subject nucleic acids have been introduced into a cell, the cell is typically incubated, normally at 37° C., sometimes under selection, for a period of about 1-24 hours in order to allow for the expression of the antibody. In one embodiment, the antibody is secreted into the supernatant of the media in which the cell is growing.

Traditionally, monoclonal antibodies have been produced as native molecules in murine hybridoma lines. In addition to that technology, the present invention provides for recombinant DNA expression of monoclonal antibodies. This allows the production of caninized, felinized, equinized, fully canine, fully feline, and fully equine antibodies, as well as a spectrum of antibody derivatives and fusion proteins in a host species of choice.

A nucleic acid sequence encoding at least one anti-IL-31 antibody, portion or polypeptide of the present invention may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., MOLECULAR CLONING, LAB. MANUAL, (Cold Spring Harbor Lab. Press, NY, 1982 and 1989), and Ausubel et al. 1993 supra, may be used to construct nucleic acid sequences which encode a monoclonal antibody molecule or antigen binding region thereof.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as anti-IL-31 peptides or antibody portions in recoverable amounts. The precise nature of the regulatory regions needed for gene expression may vary from organism to organism, as is well known in the analogous art. See, e.g., Sambrook et al., 2001 supra; Ausubel et al., 1993 supra.

The present invention accordingly encompasses the expression of an anti-IL-31 antibody or IL-31-specific polypeptide or peptide, in either prokaryotic or eukaryotic cells. Suitable hosts include bacterial or eukaryotic hosts including bacteria, yeast, insects, fungi, bird and mammalian cells either in vivo, or in situ, or host cells of mammalian, insect, bird or yeast origin. The mammalian cell or tissue may be of human, primate, hamster, rabbit, rodent, cow, pig, sheep, horse, goat, dog or cat origin, but any other mammalian cell may be used.

In one embodiment, the introduced nucleotide sequence will be incorporated into a plasmid or viral vector capable of autonomous replication in the recipient host. Any of a wide variety of vectors may be employed for this purpose. See, e.g., Ausubel et al., 1993 supra. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli(such as, for example, pBR322, ColE1, pSC101, pACYC 184, .pi.VX). Such plasmids are, for example, disclosed by Maniatis et al., 1989 supra; Ausubel et al, 1993 supra. Bacillus plasmids include pC194, pC221, pT127, etc. Such plasmids are disclosed by Gryczan, in THE MOLEC. BIO. OF THE BACILLI 307-329 (Academic Press, NY, 1982). Suitable Streptomyces plasmids include pIJ101 (Kendall et al., 169 J. Bacteriol. 4177-83 (1987)), and Streptomyces bacteriophages such as .phi.C31 (Chater et al., in SIXTH INT'L SYMPOSIUM ON ACTINOMYCETALES BIO. 45-54 (Akademiai Kaido, Budapest, Hungary 1986). Pseudomonas plasmids are reviewed in John et al., 8 Rev. Infect. Dis. 693-704 (1986); Izaki, 33 Jpn. J. Bacteriol. 729-42 (1978); and Ausubel et al., 1993 supra.

Alternatively, gene expression elements useful for the expression of cDNA encoding anti-IL-31 antibodies or peptides include, but are not limited to (a) viral transcription promoters and their enhancer elements, such as the SV40 early promoter (Okayama et al., 3 Mol. Cell. Biol. 280 (1983)), Rous sarcoma virus LTR (Gorman et al., 79 Proc. Natl. Acad. Sci., USA 6777 (1982)), and Moloney murine leukemia virus LTR (Grosschedl et al., 41 Cell 885 (1985)); (b) splice regions and polyadenylation sites such as those derived from the SV40 late region (Okayarea et al., MCB, 3: 280 (1983), and (c) polyadenylation sites such as in SV40 (Okayama et al., 1983, supra).

Immunoglobulin cDNA genes can be expressed as described by Weidle et al., 51(1) Gene 21-29 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., 1 Protein Engin. 499-505 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In one embodiment, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene can be assembled in, or inserted into, an expression vector. Recipient cells capable of expressing the chimeric immunoglobulin chain gene product are then transfected singly with an anti-IL-31 peptide or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In one embodiment, the fused genes encoding the anti-IL-31 peptide or chimeric H and L chains, or portions thereof are assembled in separate expression vectors that are then used to co-transfect a recipient cell. Alternatively the fused genes encoding the chimeric H and L chains can be assembled on the same expression vector.

For transfection of the expression vectors and production of the chimeric antibody, the recipient cell line may be a myeloma cell. Myeloma cells can synthesize, assemble and secrete immunoglobulins encoded by transfected immunoglobulin genes and possess the mechanism for glycosylation of the immunoglobulin. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

The expression vector carrying a chimeric, caninized, felinized, equinized, fully canine, fully feline, or fully equine anti-IL-31 antibody construct or IL-31-specific polypeptide or peptide (e.g., antigen-binding portion of the antibodies described herein) of the present invention can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538-1541 (1988).

Yeast can provide substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Int'l Conference on Yeast, Genetics & Molec. Biol. (Montpelier, France, 1982).

Yeast gene expression systems can be routinely evaluated for the levels of production, secretion and the stability of anti-IL-31 peptides, antibody and assembled murine and chimeric, heterochimeric, caninized, felinized, equinized, fully canine, fully feline, or fully equine antibodies, fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See Vol. II DNA Cloning, 45-66, (Glover, ed.,) IRL Press, Oxford, U K 1985).

Bacterial strains can also be utilized as hosts for the production of antibody molecules or peptides described by this invention. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of murine, chimeric, heterochimeric, caninized, felinized, equinized, fully canine, fully feline, or fully equine antibodies, fragments and regions or antibody chains encoded by the cloned immunoglobulin cDNAs in bacteria (see Glover, 1985 supra; Ausubel, 1993 supra; Sambrook, 2001 supra; Colligan et al., eds. Current Protocols in Immunology, John Wiley & Sons, NY, NY (1994-2001); Colligan et al., eds. Current Protocols in Protein Science, John Wiley & Sons, NY, NY (1997-2001).

Host mammalian cells may be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein.

Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells.

Many vector systems are available for the expression of cloned anti-IL-31 peptides H and L chain genes in mammalian cells (see Glover, 1985 supra). Different approaches can be followed to obtain complete H₂L₂ antibodies. It is possible to co-express H and L chains in the same cells to achieve intracellular association and linkage of H and L chains into complete tetrameric H₂L₂ antibodies and/or anti-IL-31 peptides. The co-expression can occur by using either the same or different plasmids in the same host. Genes for both H and L chains and/or anti-IL-31 peptides can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing anti-IL-31 peptides and/or H₂L₂ molecules via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds/components that interact directly or indirectly with the antibody molecule.

Once an antibody of the invention has been produced, it may be purified by any method known in the art for purification of an immunoglobulin molecule, for example, by chromatography (e.g., ion exchange, affinity, particularly affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

### Pharmaceutical Applications

The anti-IL-31 antibodies or IL-31-specific polypeptides or peptides of the present invention can be used for example in the treatment of pruritic and/or allergic conditions in companion animals, such as dogs, cats, and horses. In one embodiment, such polypeptides or peptides comprise the antigen-binding portion of the anti-IL-31 antibodies described herein. More specifically, the invention further provides for a pharmaceutical composition comprising a pharmaceutically acceptable carrier or diluent and, as active ingredient, an antibody or polypeptide or peptide according to the invention. The antibody can be a chimeric, heterochimeric, caninized, felinized, equinized, fully canine, fully feline, or fully equine antibody according to the present invention. Intact immunoglobulins or their binding fragments, such as Fab, are also envisioned. The antibody and pharmaceutical compositions thereof of this invention are useful for parenteral administration, e.g., subcutaneously, intramuscularly or intravenously.

Anti-IL-31 antibodies and/or IL-31-specific polypeptides and/or IL-31-specific peptides of the present invention can be administered either as individual therapeutic agents or in combination with other therapeutic agents. They can be administered alone, but are generally administered with a pharmaceutical carrier selected on the basis of the chosen route of administration and standard pharmaceutical practice.

Administration of the antibodies disclosed herein may be carried out by any suitable means, including parenteral injection (such as intraperitoneal, subcutaneous, or intramuscular injection), orally, or by topical administration of the antibodies (typically carried in a pharmaceutical formulation) to an airway surface. Topical administration to an airway surface can be carried out by intranasal administration (e.g., by use of dropper, swab, or inhaler). Topical administration of the antibodies to an airway surface can also be carried out by inhalation administration, such as by creating respirable particles of a pharmaceutical formulation (including both solid and liquid particles) containing the antibodies as an aerosol suspension, and then causing the subject to inhale the respirable particles. Methods and apparatus for administering respirable particles of pharmaceutical formulations are well known, and any conventional technique can be employed.

Oral administration may be, for example, in the form of an ingestable liquid or solid formulation.

In some desired embodiments, the antibodies are administered by parenteral injection. For parenteral administration, anti-IL-31 antibodies or polypeptides or peptides can be formulated as a solution, suspension, emulsion or lyophilized powder in association with a pharmaceutically acceptable parenteral vehicle. For example the vehicle may be a solution of the antibody or a cocktail thereof dissolved in an acceptable carrier, such as an aqueous carrier such vehicles are water, saline, Ringer's solution, dextrose solution, trehalose or sucrose solution, or 5% serum albumin, 0.4% saline, 0.3% glycine and the like. Liposomes and nonaqueous vehicles such as fixed oils can also be used. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjustment agents and the like, for example sodium acetate, sodium chloride, potassium chloride, calcium chloride, sodium lactate, etc. The concentration of antibody in these formulations can vary widely, for example from less than about 0.5%, usually at or at least about 1% to as much as 15% or 20% by weight and will be selected primarily based on fluid volumes, viscosities, etc., in accordance with the particular mode of administration selected. The vehicle or lyophilized powder can contain additives that maintain isotonicity (e.g., sodium chloride, mannitol) and chemical stability (e.g., buffers and preservatives). The formulation is sterilized by commonly used techniques.

Actual methods for preparing parenterally administrable compositions will be known or apparent to those skilled in the art and are described in more detail in, for example, REMINGTON'S PHARMA. SCI. (15th ed., Mack Pub. Co., Easton, Pa., 1980).

The antibodies of this invention can be lyophilized for storage and reconstituted in a suitable carrier prior to use. This technique has been shown to be effective with conventional immune globulins. Any suitable lyophilization and reconstitution techniques can be employed. It will be appreciated by those skilled in the art that lyophilization and reconstitution can lead to varying degrees of antibody activity loss and that use levels may have to be adjusted to compensate.

The compositions containing the present antibodies or a cocktail thereof can be administered for prevention of recurrence and/or therapeutic treatments for existing disease. Suitable pharmaceutical carriers are described in the most recent edition of REMINGTON'S PHARMACEUTICAL SCIENCES, a standard reference text in this field of art.

In therapeutic application, compositions are administered to a subject already suffering from a disease, in an amount sufficient to cure or at least partially arrest or alleviate the disease and its complications. An amount adequate to accomplish this is defined as a “therapeutically effective dose” or a “therapeutically effective amount”. Amounts effective for this use will depend upon the severity of the disease and the general state of the subject's own immune system, but generally range from about 0.1 mg antibody per kg body weight to about 15 mg antibody per kg body weight, preferably about 0.3 mg antibody per kg of body weight to about 12 mg of antibody per kg of body weight. In one embodiment, the therapeutically effective amount will provide at least one month duration of efficacy with a dose up to 12 mg/kg of body weight. In view of the minimization of extraneous substances and the lower probability of “foreign substance” rejections which are achieved by the present canine-like, feline-like, and equine-like antibodies of this invention, it may be possible to administer substantial excesses of these antibodies.

The dosage administered will, of course, vary depending upon known factors such as the pharmacodynamic characteristics of the particular agent, and its mode and route of administration; age, health, and weight of the recipient; nature and extent of symptoms kind of concurrent treatment, frequency of treatment, and the effect desired.

As a non-limiting example, treatment of IL-31-related pathologies in dogs, cats, or horses can be provided as a biweekly or monthly dosage of anti-IL-31 antibodies of the present invention in the dosage range described above.

Example antibodies for canine, feline, or equine therapeutic use are high affinity (these may also be high avidity) antibodies, and fragments, regions and derivatives thereof having potent in vivo anti-IL-31 activity, according to the present invention. The antibody fragments and regions may be alternatively referred to herein as polypeptides or peptides of the present invention which include the antigen-binding portion of the anti-II-31 antibodies.

Single or multiple administrations of the compositions can be carried out with dose levels and pattern being selected by the treating veterinarian. In any event, the pharmaceutical formulations should provide a quantity of the antibody or antibodies of this invention sufficient to effectively treat the subject.

### Diagnostic Applications

The present invention also provides the above anti-IL-31 antibodies, polypeptides, and/or peptides for use in diagnostic methods for detecting IL-31 in companion animals known to be or suspected of having a puritic and/or allergic condition.

Anti-IL-31 antibodies, polypeptides, and/or peptides of the present invention are useful for immunoassays which detect or quantitate IL-31, or anti-IL-31 antibodies, in a sample. An immunoassay for IL-31 typically comprises incubating a clinical or biological sample in the presence of a detectably labeled high affinity (or high avidity) anti-IL-31 antibody, polypeptide, or peptide of the present invention capable of selectively binding to IL-31, and detecting the labeled polypeptide, peptide or antibody which is bound in a sample. Various clinical assay procedures are well known in the art. See, e.g., IMMUNOASSAYS FOR THE 80'S (Voller et al., eds., Univ. Park, 1981). Such samples include tissue biopsy, blood, serum, and fecal samples, or liquids collected from animal subjects and subjected to ELISA analysis as described below.

In some embodiments, the binding of antigen to antibody is detected without the use of a solid support. For example, the binding of antigen to antibody can be detected in a liquid format.

In other embodiments, an anti-IL-31 antibody, polypeptide, or peptide can, for example, be fixed to nitrocellulose, or another solid support which is capable of immobilizing cells, cell particles or soluble proteins. The support can then be washed with suitable buffers followed by treatment with the detectably labeled IL-31-specific polypeptide, peptide or antibody. The solid phase support can then be washed with the buffer a second time to remove unbound polypeptide, peptide or antibody. The amount of bound label on the solid support can then be detected by known method steps.

“Solid phase support” or “carrier” refers to any support capable of binding polypeptide, peptide, antigen, or antibody. Well-known supports or carriers, include glass, polystyrene, polypropylene, polyethylene, polyvinylidenefluoride (PVDF), dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to IL-31 or an anti-IL-31 antibody. Thus, the support configuration can be spherical, as in a bead, or cylindrical, as in the inside surface of a test tube, or the external surface of a rod. Alternatively, the surface can be flat, such as a sheet, culture dish, test strip, etc. For example, supports may include polystyrene beads. Those skilled in the art will know many other suitable carriers for binding antibody, polypeptide, peptide or antigen, or can ascertain the same by routine experimentation.

Well known method steps can determine binding activity of a given lot of anti-IL-31 polypeptide, peptide and/or antibody. Those skilled in the art can determine operative and optimal assay conditions by routine experimentation.

Detectably labeling an IL-31-specific polypeptide, peptide and/or antibody can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the IL-31-specific antibodies of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

By radioactively labeling the IL-31-specific antibodies, it is possible to detect IL-31 through the use of a radioimmunoassay (RIA). See Work et al., LAB. TECHNIQUES & BIOCHEM. 1N MOLEC. Bio. (No. Holland Pub. Co., NY, 1978). The radioactive isotope can be detected by such means as the use of a gamma counter or a scintillation counter or by autoradiography. Isotopes which are particularly useful for the purpose of the present invention include: ³H, ¹²⁵I, ¹³¹I, ³⁵S, ¹⁴C, and ¹²⁵I.

It is also possible to label the IL-31-specific antibodies with a fluorescent compound. When the fluorescent labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The IL-31-specific antibodies can also be delectably labeled using fluorescence-emitting metals such a ¹²⁵Eu, or others of the lanthanide series. These metals can be attached to the IL-31-specific antibody using such metal chelating groups as diethylenetriaminepentaacetic acid (DTPA) or ethylenediamine-tetraacetic acid (EDTA).

The IL-31-specific antibodies also can be detectably labeled by coupling to a chemiluminescent compound. The presence of the chemiluminescently labeled antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound can be used to label the IL-31-specific antibody, portion, fragment, polypeptide, or derivative of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the IL-31-specific antibody, portion, fragment, polypeptide, or derivative can be accomplished by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

For the purposes of the present invention, the IL-31 which is detected by the above assays can be present in a biological sample. Any sample containing IL-31 may be used. For example, the sample is a biological fluid such as, for example, blood, serum, lymph, urine, feces, inflammatory exudate, cerebrospinal fluid, amniotic fluid, a tissue extract or homogenate, and the like. The invention is not limited to assays using only these samples, however, it being possible for one of ordinary skill in the art, in light of the present specification, to determine suitable conditions which allow the use of other samples.

In situ detection can be accomplished by removing a histological specimen from an animal subject, and providing the combination of labeled antibodies of the present invention to such a specimen. The antibody (or portion thereof) may be provided by applying or by overlaying the labeled antibody (or portion) to a biological sample. Through the use of such a procedure, it is possible to determine not only the presence of IL-31 but also the distribution of IL-31 in the examined tissue. Using the present invention, those of ordinary skill will readily perceive that any of a wide variety of histological methods (such as staining procedures) can be modified in order to achieve such in situ detection.

The antibody, fragment or derivative of the present invention can be adapted for utilization in an immunometric assay, also known as a “two-site” or “sandwich” assay. In a typical immunometric assay, a quantity of unlabeled antibody (or fragment of antibody) is bound to a solid support that is insoluble in the fluid being tested and a quantity of detectably labeled soluble antibody is added to permit detection and/or quantification of the ternary complex formed between solid-phase antibody, antigen, and labeled antibody.

The antibodies may be used to quantitatively or qualitatively detect the IL-31 in a sample or to detect presence of cells that express the IL-31. This can be accomplished by immunofluorescence techniques employing a fluorescently labeled antibody (see below) coupled with fluorescence microscopy, flow cytometric, or fluorometric detection. For diagnostic purposes, the antibodies may either be labeled or unlabeled. Unlabeled antibodies can be used in combination with other labeled antibodies (second antibodies) that are reactive with the antibody, such as antibodies specific for canine or feline immunoglobulin constant regions. Alternatively, the antibodies can be directly labeled. A wide variety of labels may be employed, such as radionuclides, fluors, enzymes, enzyme substrates, enzyme cofactors, enzyme inhibitors, ligands (particularly haptens), etc. Numerous types of immunoassays, such as those discussed previously are available and are well known to those skilled in the art.

In one embodiment, the diagnostic method for detecting IL-31 is a lateral flow immunoassay test. This is also known as the immunochromatographic assay, Rapid ImmunoMigration (RIM™) or strip test. Lateral flow immunoassays are essentially immunoassays adapted to operate along a single axis to suit the test strip format. A number of variations of the technology have been developed into commercial products, but they all operate according to the same basic principle. A typical test strip consists of the following components: (1) sample pad—an absorbent pad onto which the test sample is applied; (2) conjugate or reagent pad—this contains antibodies specific to the target analyte conjugated to colored particles (usually colloidal gold particles, or latex microspheres); (3) reaction membrane—typically a hydrophobic nitrocellulose or cellulose acetate membrane onto which anti-target analyte antibodies are immobilized in a line across the membrane as a capture zone or test line (a control zone may also be present, containing antibodies specific for the conjugate antibodies); and (4) wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones.

There are two main types of lateral flow immunoassay used in microbiological testing: double antibody sandwich assays and competitive assays. In the double antibody sandwich format, the sample migrates from the sample pad through the conjugate pad where any target analyte present will bind to the conjugate. The sample then continues to migrate across the membrane until it reaches the capture zone where the target/conjugate complex will bind to the immobilized antibodies producing a visible line on the membrane. The sample then migrates further along the strip until it reaches the control zone, where excess conjugate will bind and produce a second visible line on the membrane. This control line indicates that the sample has migrated across the membrane as intended. Two clear lines on the membrane is a positive result. A single line in the control zone is a negative result. Competitive assays differ from the double antibody sandwich format in that the conjugate pad contains antibodies that are already bound to the target analyte, or to an analogue of it. If the target analyte is present in the sample it will therefore not bind with the conjugate and will remain unlabelled. As the sample migrates along the membrane and reaches the capture zone an excess of unlabelled analyte will bind to the immobilized antibodies and block the capture of the conjugate, so that no visible line is produced. The unbound conjugate will then bind to the antibodies in the control zone producing a visible control line. A single control line on the membrane is a positive result. Two visible lines in the capture and control zones is a negative result. However, if an excess of unlabelled target analyte is not present, a weak line may be produced in the capture zone, indicating an inconclusive result. There are a number of variations on lateral flow technology. The capture zone on the membrane may contain immobilized antigens or enzymes—depending on the target analyte—rather than antibodies. It is also possible to apply multiple capture zones to create a multiplex test. For example, commercial test strips able to detect both EHEC Shiga toxins ST1 and ST2 separately in the same sample have been developed.

Importantly, the antibodies of the present invention may be helpful in diagnosing a pruritic and/or allergic in dogs, cats, or horses. More specifically, the antibody of the present invention may identify the overexpression of IL-31 in companion animals. Thus, the antibody of the present invention may provide an important immunohistochemistry tool. In one embodiment, an assay design is conceived here whereby an IL-31 mimotope (peptide) is used to capture an antibody of the present invention that is labeled for detection in an assay. This captured antibody would have an affinity to the attached mimotope that is lower that the affinity of native circulating IL-31 in a host species. In this embodiment, incubation of the fluid derived from the host species is incubated with the labeled antibody: mimotope complex that is tethered to a solid surface. The presence of IL-31 in the test fluid derived from the host species will have a higher affinity to the antibody, thus liberating the labeled antibody from the solid surface where it can be removed during wash steps. The level of IL-31 in the test fluid can thus be correlated to the lack of signal that appears on the mimotope-bound surface. It is conceived that such an assay would have utility to measure IL-31 in a research or clinical setting for use as a diagnostic test.

The antibodies of the present invention may be used on antibody arrays, highly suitable for measuring gene expression profiles.

### Kits

Also included within the scope of the present invention are kits for practicing the subject methods. The kits at least include one or more of the antibodies of the present invention, a nucleic acid encoding the same, or a cell containing the same. In one embodiment, an antibody of the present invention may be provided, usually in a lyophilized form, in a container. The antibodies, which may be conjugated to a label or toxin, or unconjugated, are typically included in the kits with buffers, such as Tris, phosphate, carbonate, etc., stabilizers, biocides, inert proteins, e.g., serum albumin, or the like. Generally, these materials will be present in less than 5% wt. based on the amount of active antibody, and usually present in total amount of at least about 0.001% wt. based again on the antibody concentration. Frequently, it will be desirable to include an inert extender or excipient to dilute the active ingredients, where the excipient may be present in from about 1% to 99% wt. of the total composition. Where a second antibody capable of binding to the primary antibody is employed in an assay, this will usually be present in a separate vial. The second antibody is typically conjugated to a label and formulated in an analogous manner with the antibody formulations described above. The kit will generally also include a set of instructions for use.

In one embodiment, a kit according to the present invention is a test strip kit (lateral flow immunoassay kit) useful for detecting canine, feline, or equine IL-31 protein in a sample. Such a test strip will typically include a sample pad onto which the test sample is applied; a conjugate or reagent pad containing an antibody specific to canine, feline, or equine IL-31, wherein the antibody is conjugated to colored particles (usually colloidal gold particles); a reaction membrane onto which anti-IL-31 antibodies are immobilized in a line across the membrane as a capture zone or test line (a control zone may also be present, containing antibodies specific for the conjugate antibodies); and a further absorbent pad designed to draw the sample across the reaction membrane by capillary action and collect it. The test strip kit will generally also include directions for use.

Methods of Improving the Consistency and/or Quality of a Feline or Canine Antibody

Such methods are described above in the Summary of the Invention section, as well as in the Examples and figures of the present application.

The present inventors have surprisingly discovered that the removal, or modification of the c-terminal end of the kappa light chain constant from those animal species whose native germline encodes additional residues beyond the terminal light chain Cysteine is beneficial to both the production of homogeneous recombinant antibody for these species and beneficial to the amount of antibody produced from a stable cell line (e.g., yield improvement). The results described herein support that additional amino acid residues beyond the terminal cysteine in the kappa light chain of feline (and canine) is detrimental to efficient pairing with the heavy chain leading to mispairing and poor production of the antibody.

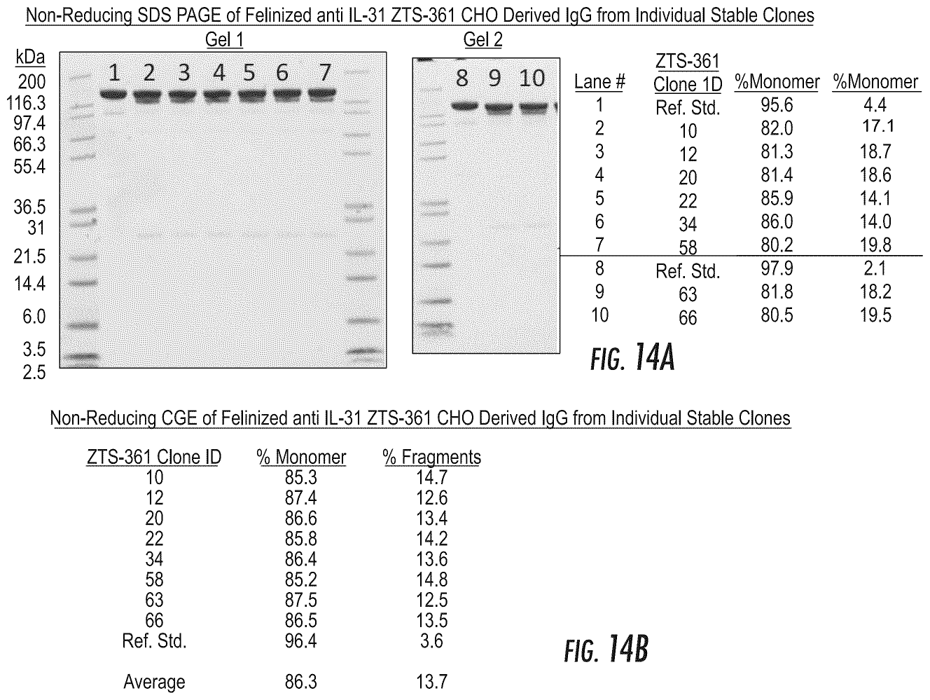

In one embodiment, the present invention provides a method of improving the consistency and/or quality of a feline antibody. This method includes expressing nucleotide sequence encoding a feline IgG kappa light chain and nucleotide sequence encoding a feline IgG heavy chain in a host cell to produce a feline antibody, wherein the nucleotide sequence encoding the feline IgG kappa light chain comprises a kappa light chain constant nucleotide sequence in which sequence encoding a C-terminal QRE sequence otherwise present in a wild-type feline IgG kappa light chain constant region has been modified and/or deleted. With reference to the C-terminal amino acid residues of the feline Ig Kappa light chain constant domain shown in FIG. 15, in one embodiment, the present invention provides for removal of the c-terminal “QRE” that immediately follows CYS107 of the feline light chain kappa sequence of SEQ ID NO: 175. This modification was found to improve the production of monomeric recombinant feline IgG. However, the present invention is not limited in this regard. For example, even three additional amino acids added contiguously to the c-terminal end of the Cysteine at position 107 in place of the native QRE may be tolerated if the amino acids have a minimal electrostatic charge influence.

The present invention further provides a method of improving the consistency and/or quality of a canine antibody. This method includes expressing nucleotide sequence encoding a canine IgG kappa light chain and nucleotide sequence encoding a canine IgG heavy chain in a host cell to produce a canine antibody, wherein the nucleotide sequence encoding the canine IgG kappa light chain comprises a kappa light chain constant nucleotide sequence in which sequence encoding a C-terminal QRVD sequence otherwise present in a wild-type canine IgG kappa light chain constant region has been modified and/or deleted. With reference to the C-terminal amino acid residues of the canine Ig Kappa light chain constant domain shown in FIG. 15, in one embodiment, the present invention provides for removal of the c-terminal “QRVD” that immediately follows CYS105 of the canine light chain kappa sequence of SEQ ID NO: 194. However, the present invention is not limited in this regard. For example, even three additional amino acids added contiguously to the c-terminal end of the Cysteine at position 105 in place of the native QRVD may be tolerated if the amino acids have a minimal electrostatic charge influence.

The results herein clearly demonstrate that the above methods apply to structurally disparate antibodies which recognize completely distinct targets and therefore these modifications will likely be applicable to the broad genus of feline antibodies, including but not limited to anti-IL31 and anti-NGF antibodies, as well as other mammalian antibodies possessing additional C-terminal amino acids on the kappa light chain constant region. While not wishing to be bound by any one theory, this light chain modification appears to result in a higher fidelity of immunoglobulin chain pairing during the induced production from stable CHO cell lines resulting in a higher amount of monomeric IgG and potentially a higher overall antibody yield. Both of these attributes are highly desirable from the standpoint of manufacturing commercial grade antibody therapeutics.

In one embodiment, any of the anti-IL-31 antibodies disclosed herein can comprise the kappa light chain constant deletions and/or modifications disclosed herein. For example, in one embodiment, a feline antibody according to the present invention comprises a kappa light chain constant region wherein the “QRE” normally present at the c-terminal of the kappa light chain constant region has been removed and optionally replaced with up to three additional amino acids which have a minimal electrostatic charge influence. In a particular embodiment, a feline antibody according to the present invention comprises a feline kappa light chain having the sequence:

(SEQ ID NO: 186)
RSDAQPSVFLFQPSLDELHTGSASIVCILNDFYPKEVNVKWKVDGVVQN
KGIQESTTEQNSKDSTYSLSSTLTMSSTEYQSHEKFSCEVTHKSLASTL
VKSFQRSEC
or
a variant thereof.

The invention will now be described further by the non-limiting examples below. In the example section below and in the Figures, any data presented for antibodies containing “11E12” in their designation is for purposes of comparison with the antibodies of the present invention.

### Examples

1.1. Production of Canine Interleukin 31 (cIL-31) from Chinese Hamster Ovary (CHO) Cells

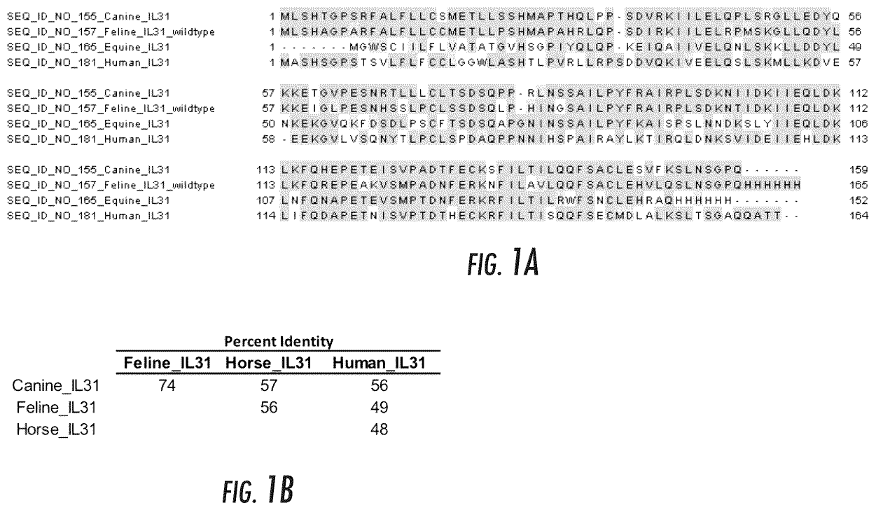

The Interleukin 31 protein varies in amino acid sequence conservation among homologous species (FIGS. 1A and 1B) but is believed to have common structural architecture with other members of the type I cytokine family (Boulay et al. 2003, Immunity. August; 19(2):159-632003; Dillon et al. 2004 Nat Immunol. July; 5(7):752-60). This up-down bundle topology is significant to the mode of receptor recognition shared by these cytokines (Dillon et al. supra, Cornelissen et al. 2012 Eur J Cell Biol. June-July; 91(6-7):552-66). With variation in IL-31 protein sequence identities between different species, it is not possible to predict if antibodies raised against one species will cross-react with others given different epitope propensities and local amino acid compositions. As a consequence, multiple forms of IL-31 protein were considered for this work representing multiple species and expression systems. The canine IL-31 protein (cIL-31) was produced to use as an immunogen and a reagent to test affinity and potency of antibody hits.

Recombinant cIL-31 was created in CHO cells using the CHROMOS ACE (Artificial Chromosome Expression) system (Chromos Molecular Systems, Inc., Burnaby, British Columbia) to generate the secreted canine IL-31 protein having the sequence of (SEQ ID NO: 155; Canine_IL31), the corresponding nucleotide sequence for which is (SEQ ID NO: 156; Canine_IL31). Conditioned medium from 400 ml of cell culture (CHO cell line) was obtained and dialyzed against 10 volumes of QA buffer (20 mM Tris pH 8.0, 20 mM NaCl) for 4.5 hours. Dialyzed medium was 0.2 μm filtered and loaded at 1 ml/min onto a SOURCE™ Q column (GE Healthcare, Uppsala, Sweden) pre-equilibrated with QA buffer. Protein was eluted using a multi-step linear gradient. The majority of cIL-31 remained in the flow through (FT) fraction, a small amount of cIL-31 eluted early in the gradient. Identity of the protein was previously confirmed by Western immunoblotting, and Mass-Spectrometry (MS) analysis of a tryptic digest. Protein in the FT fraction was concentrated 4-5 fold and dialyzed overnight against Phosphate Buffered Saline (PBS) at 4° C. Stability of the protein was checked following dialysis into PBS. No precipitation was observed, and no proteolysis was observed after several days at 4° C. De-glycosylation experiments using N-glycosidase F resulted in the protein condensing down to a single band of ˜15 kDa on SDS-PAGE. Protein concentration was determined using a bicinchoninic assay (BCA assay) with Bovine Serum Albumin (BSA) as a standard (ThermoFisher Scientific, Inc., Rockford, IL). The protein solution was split into aliquots, snap frozen (liquid N₂) and stored at −80° C.

1.2. Transient Expression of Wildtype and Mutant Feline Interleukin 31 (fIL-31) from CHO Cells

To aid in the identification of antibodies with the appropriate epitope binding property, wildtype and mutant feline IL-31 proteins were expressed in a mammalian expression system for production, purification, and assessment in affinity and cell-based assays. The binding site of antibody 11E12 on IL-31 was described previously (U.S. Pat. No. 8,790,651 to Bammert, et al.). Characterization of the novel binding site on IL-31 recognized by antibody 15H05 is described herein. The wildtype designation is full length feline IL-31 protein with no changes to the native amino acid residues. Mutant proteins were designated by their corresponding antibodies name (11E12 and 15H05) referring to mutations in amino acids in the IL-31 protein that (when altered) affect the binding to each respective antibody. Identification of the appropriate mutations required for the feline IL-31 15H05 protein are described below in section 1.10. The objective was to change amino acids in the IL-31 epitope and observe a loss of binding phenotype to each respective antibody. A comparison can then be made during screening to see if new candidate antibodies bind to the wildtype protein and not to the mutant. New antibody hits can then be binned according to binding to the same or similar epitope as antibody 11E12 or 15H05.

Expression constructs were codon optimized and synthesized for expression in Chinese Hamster Ovarian (CHO) cells. The synthesized genes were cloned into pD2529 (ATUM vector) for transient expression use. Wildtype feline IL-31 protein is represented by (SEQ ID NO: 157; Feline_IL31_wildtype), the corresponding nucleotide sequence for which is (SEQ ID NO: 158; Feline_IL31_wildtype). Mutant feline IL-31 11E12 protein is represented by (SEQ ID NO: 161; Feline_IL31_11E12_mutant), the corresponding nucleotide sequence for which is (SEQ ID NO: 162; Feline_IL31_11E12_mutant). Mutant feline IL-31 15H05 protein is represented by (SEQ ID NO: 163; Feline_IL31_15H05_mutant), the corresponding nucleotide sequence for which is (SEQ ID NO: 164; Feline_IL31_15H05_mutant). Recombinant feline IL-31 proteins were expressed in ExpiCHO-S™ cells (ThermoFisher Scientific, Inc., Rockford, IL) by following the manufacturer's maximum titer protocol for transient CHO expression. Twelve days post transfection the cells were centrifuged and filtered to capture the secreted protein in the conditioned media. For each construct (wild type and mutants), 120 mL of conditioned media (from CHO cell culture, 0.2 μm filtered) was adjusted to 30 mS/cm with the addition of NaCl, 5 mM imidazole, and pH 7.4. Each media sample was combined with 5 mL of HisPur Cobalt resin (ThermoFisher Scientific, Inc., Rockford, IL) which had been equilibrated with 5 mM imidazole, 20 mM sodium phosphate, 300 mM NaCl, pH 7.4. Each sample and resin was allowed to mix at 4° C., overnight. Resins were collected (and separated from the unbound fraction) by pouring through BioRad Econcolumns (Bio-Rad, Hercules, CA). Resins were washed with 5×5 mL of buffer (as above) and then eluted with 5×5 mL of 500 mM imidazole in the same buffer. Fractions were evaluated by SDS-PAGE. Concentration was measured by a BCA protein assay using standard methods.

1.3. Production of Feline Interleukin 31 (fIL-31) from E. coli

Recombinant feline IL-31 protein was generated in an E. coli expression host to use as an assay reagent and for in vivo challenge studies to induce a pruritic response in cats. The gene representing feline IL-31 was synthesized for optimal expression in E. coli. Expression constructs were created with the full length feline IL-31 gene containing an N-terminal 6-His tag for detection and purification. This feline IL-31 protein is represented by (SEQ ID NO: 159; Feline_IL-31_E_coli), the corresponding nucleotide sequence for which is (SEQ ID NO: 160; Feline_IL-31_E_coli). Sequence confirmed plasmids were used to transform E. coli BL21 DE3 (Invitrogen Corp., Carlsbad, CA) and subsequent protein expression carried out.

Cell paste (262.3 g) from E. coli was lysed as follows: The cell paste was resuspended in 500 mL of 50 mM Tris pH 8, filtered through a stainless mesh filter to remove particles, and then lysed via two passes through a microfluidizer at 1300 psi. The lysate (˜1200 mL volume) was divided into four bottles, centrifuged at 12,000 g for 20 minutes at 10° C. The supernatant was decanted and discarded. Each pellet was washed by suspension in 300 mL of 5 mM EDTA, 0.5% Triton X-100, pH 9.0, and then centrifugation at 12,000 g, 50 minutes, at 10° C. The supernatant was decanted and discarded. The washed pellets were stored at −20° C. until folding and isolation.

Prior to isolation, one of the pellets was washed with water to remove the residual detergent, and then centrifuged at 10,000 g, 20 minutes, at 4° C. Again, the supernatant was decanted. Finally, the washed pellet was solubilized in 60 mL of 50 mM sodium phosphate, 300 mM NaCl, 6 M guanidine-HCl, 5 mM imidazole, pH 7.4. The pellet was allowed to mix at room temperature for approximately 25 minutes before centrifuging again at 10,000 g, 20 minutes, and 4° C. This time, the supernatant was decanted and kept for further processing. The pellet (resuspended in water to original volume) was set aside for SDS-PAGE only. Crude IMAC (immobilized metal affinity chromatography) was performed to increase purity prior to folding. In this case, 15 mL of Ni-NTA Superflow (Qiagen Inc, Germantown, MD, Cat #30450, pre-equilibrated in the same buffer) was added to the clarified supernatant and allowed to mix at room temperature for approximately 90 minutes. The unbound fraction was decanted and set aside for SDS-PAGE. The IMAC resin was washed with 5 mM imidazole, 50 mM sodium phosphate, 300 mM NaCl, 6 M guanidine-HCl, pH 7.4 (same as solubilization buffer). The resin was eluted with (first 7.5 mL and then multiples of 15 mL, monitoring protein elution by Bradford assay) 200 mM imidazole, 50 mM sodium phosphate, 300 mM NaCl, 6 M guanidine-HCl, pH 7.4. Elution fractions containing protein (as per Bradford) were pooled (125 mL) for further processing.

The IL-31 protein was folded as follows. The IL-31 was reduced by the addition of dithiothreitol to a final of 10 mM, and allowed to mix at room temperature for 2 hours. The diluted sample was then diluted drop-wise into 2500 mL (20× volume) of PBS+1 M NaCl with rapid stirring.

The theoretical concentration of urea should have been approximately 0.4 M at this point. The remainder of the urea was removed slowly by dialysis against 3 exchanges (4 L each) of PBS, at 4° C. overnight. Following dialysis, the sample was 0.2 μm filtered to remove any unfolded/precipitated protein.

The sample was further purified by a second round of IMAC, this time with a linear gradient elution. Fifteen mL of Ni-NTA Superflow resin was added to the sample and allowed to bind batch-wise by stirring (with a suspended stir bar) overnight at 4° C. Again, the unbound fraction was decanted and set aside. The Ni-NTA Superflow resin was packed in an XK16 column (GE Healthcare Lifesciences, Marlborough, MA) and hooked up to an AKTA brand chromatography system (GE Healthcare Lifesciences, Marlborough, MA). The column was then washed with 50 mM Tris, 300 mM NaCl, pH 8.2 and the eluted via a 150 mL linear gradient from 0 to 500 mM imidazole, each in wash buffer. Fractions were analyzed by SDS-PAGE. Fractions having sufficient purity of IL-31 were pooled and buffer exchanged again by dialysis against 3 exchanges (2 L each) of PBS, at 4° C., overnight. Finally, the folded and purified sample was collected from dialysis, sterile filtered, concentration measured aliquoted, snap-froze in a dry-ice/isopropanol bath, and stored at −80° C.

### 1.4. Method to Determine Affinity of Anti-IL-31 Antibodies for IL-31 Using Surface Plasmon Resonance

The affinity with which candidate mAbs bind canine and feline IL-31 was determined using surface plasmon resonance (SPR) on a Biacore system (Biocore Life Sciences (GE Healthcare), Uppsala, Sweden). To avoid affinity differences associated with differential surface preparation that can occur when immobilizing antibodies to surfaces; a strategy was employed where IL-31 was directly conjugated to the surface. Immobilization was obtained by amine coupling 5 μg/mL IL-31 using N-hydroxysuccinimide (NHS)/1-Ethyl-3-(3-dimethylaminopropyl) carbodiimide (EDC) chemistry. Chips were quenched with ethanolamine and the affinity with which all candidate mAbs bound to the immobilized IL-31 was evaluated. All curves were fit to a 1:1 model. Affinity constants (KD) less than 1×10⁻¹¹ M (1E-11 M) are below the lower limit of quantitation of detection for the instrument. Results for affinity measurements are described herein.

1.5. Method to Determine Potency of Anti-IL-31 Antibodies Assessed by Inhibition of Canine and Feline IL-31 Induced pSTAT3 Signaling in Canine and Feline Macrophage Cells

To identify candidates with inhibitory activity, antibodies were assessed for their ability to affect IL-31-mediated STAT3 phosphorylation in either a canine or feline cell-based assay. STAT3 phosphorylation was determined in canine DH-82 (ATCC© CRL-10389™) or feline FCWF4 macrophage-like cells (ATCC CRL-2787). DH82 and FCWF4 cells were primed with canine interferon gamma (R&D Systems, Minneapolis, MN) at 10 ng/mL for 24 hours or feline interferon gamma (R&D Systems, Minneapolis, MN) at 125 ng/mL for 96 hours, respectively, to increase receptor expression. Both cell types were serum starved for 2 hours prior to IL-31 and mAb treatment. Using two independent methods, all candidate mAbs were evaluated for their ability to inhibit either 1 μg/mL canine or 0.2 μg/mL feline IL-31 induced STAT3 phosphorylation. Assays were also run to demonstrate cross-reactivity of canine and feline cytokines and cross-functionality of the antibodies ability to inhibit signaling in both species. To ensure complex formation, a one hour co-incubation of mAb and IL-31 cytokine prior to cell stimulation was completed. IL-31 cell stimulation was carried out for five minutes. STAT3 phosphorylation was measured using AlphaLISA SureFire ULTRA™ technology (Perkin Elmer, Waltham, MA). In the case where antibody concentration and purity are unknown, hybridoma supernatants were qualitatively measured for their ability to inhibit STAT3 phosphorylation following a 1 hour co-incubation with 1 mg/ml canine or 0.2 mg/ml feline IL-31. The potency of individual monoclonal antibodies defined by their ability to inhibit IL-31 mediated STAT3 phosphorylation in these assays was considered the key selection criteria for further advancement of select antibodies. The term potency refers to the IC50 value calculated from these assays and is the concentration of the antibody where signaling induced by IL-31 is reduced to one half its maximal value. Increased potency described herein correlates to a lower IC50 value.

### 1.6. Identification of Mouse and Canine Monoclonal Antibodies Recognizing Canine and Feline Interleukin 31

Mice and dogs were immunized with recombinant canine IL-31 (SEQ ID No. 155) for the purpose of identifying antibodies. Serum antibody titers from immunized animals were determined using an enzyme linked immunosorbent assay (ELISA). Canine or feline IL-31 (50 ng/well) was immobilized to polystyrene microplates and used as a capture antigen. Serum from immunized animals was diluted in phosphate buffered saline with 0.05% tween-20 (PBST). The presence of anti-IL-31 antibodies was detected with an appropriate secondary HRP labeled antibody. Following addition of a chromogenic substrate (SureBlue Reserve TMB 1-Component Microwell Peroxidase Substrate, KPL, Inc., Gaithersburg, MD) and a ten minute incubation at room temperature (RT) the reaction was stopped with the addition of 100 μL of 0.1 N HCl. The absorbance of each well was determined at an optical density (OD) of 450 nm. Antibodies were selected for their ability to bind canine and feline IL-31 using an ELISA. In some cases, further characterization was performed at the time of selection using an ELISA with a mutant form of the feline IL-31 protein as a capture antigen. Cells producing antibodies with desired binding and inhibitory properties were chosen for sequence analysis of RNA transcripts representing the variable heavy (VH) and variable light (VL) IgG chains.

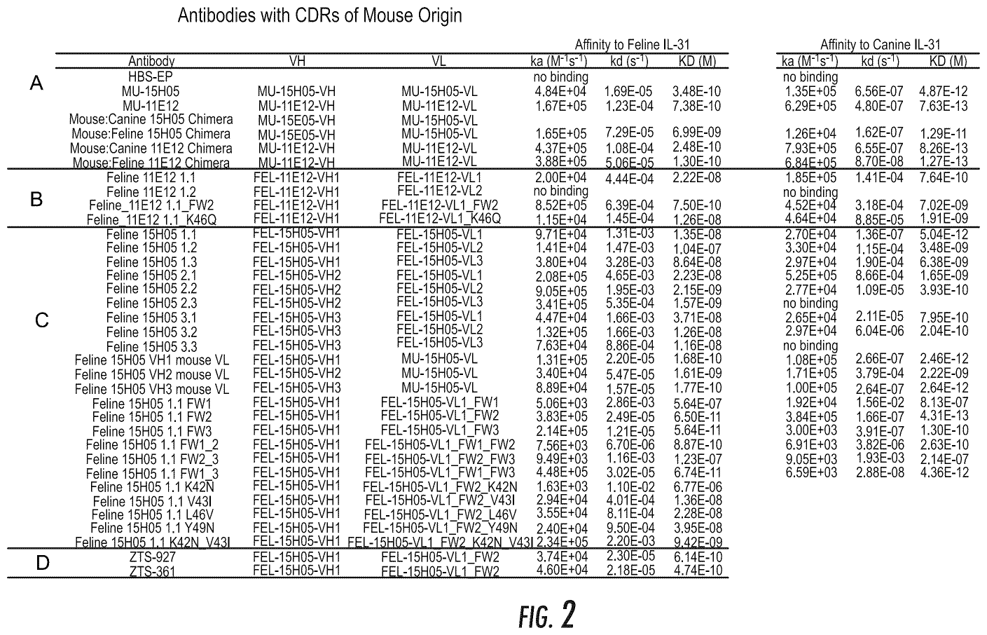

In the case of mouse antibodies, donor splenocytes from a single responsive CF-1 mouse were used for fusion and hybridoma supernatants were screened for antibodies that bind to either canine or feline IL-31 proteins by ELISA. This resulted in the identification of a single mouse antibody, Mu-15H05, having a sub-nanomolar affinity to both species of IL-31 (FIG. 2, Section A). Mouse anti IL-31 15H05 was further subcloned to generate a hybridoma producing homogeneous antibody and for sequencing of the variable heavy and light chains. The mouse anti IL-31 variable sequences determined for antibody 15H05 are as follows, 15H05 variable heavy chain (SEQ ID NO: 67; MU-15H05-VH), the corresponding nucleotide sequence for which is (SEQ ID NO: 68; MU-15H05-VH), 15H05 variable light chain (SEQ ID NO: 69; MU-15H05-VL), the corresponding nucleotide sequence for which is (SEQ ID NO: 70; MU-15H05-VL). In addition to mouse antibody 15H05, further consideration was given to mouse-derived antibody 11E12 that was previously described in U.S. Pat. No. 8,790,651 to Bammert, et al. Described herein are data showing the ability of antibody 11E12 to bind both canine and feline IL-31 proteins with high affinity. The ability of 11E12 to bind feline IL-31 made this antibody a suitable candidate for felinization and potential therapeutic use in cats. The mouse anti IL-31 variable sequences previously determined for antibody 11E12 are as follows, 11E12 variable heavy chain (SEQ ID NO: 71; MU-11E12-VH), the corresponding nucleotide sequence for which is (SEQ ID NO: 72; MU-11E12-VH), 11E12 variable light chain (SEQ ID NO: 73; MU-11E12-VL), the corresponding nucleotide sequence for which is (SEQ ID NO: 74; MU-11E12-VL).

Dogs having elevated anti IL-31 titers following vaccination were selected for analysis of B-cell populations producing antibodies with desired phenotypes. B-cells were derived from PBMCs, bone marrow, spleen, or lymph nodes for further analysis. Single B-cells were segregated into individual wells and assayed for presence of secreted IgGs capable of binding wildtype, 11E12 mutant, and 15H05 mutant forms of canine IL-31 (AbCellera, Vancouver, BC) using methods described in US2012/0009671A1, US2016/0252495A1, U.S. Pat. No. 9,188,593, WO 2015/176162 A9, and WO 2016/123692 A1.

This screening strategy is based on known regions of the IL-31 protein that are critical for binding and signal transduction through its co-receptor complex. Selection of these mutant proteins for screening is described in section 1.2 of this application. Sequencing of the variable heavy and light IgG domains was carried following an RT-PCR reaction from individual candidate B-cells. These screens resulted in the identification of nine canine antibodies selected for further evaluation. These canine anti IL-31 variable sequences are as follows, ZIL1 variable heavy chain (SEQ ID NO:75; CAN-ZIL1 VH), the corresponding nucleotide sequence for which is (SEQ ID NO: 76; CAN-ZIL1_VH), ZIL1 variable light chain (SEQ ID NO: 77; CAN-ZIL1_VL), the corresponding nucleotide sequence for which is (SEQ ID NO: 78; CAN-ZIL1_VL); ZIL8 variable heavy chain (SEQ ID NO:79; CAN-ZIL8_VH), the corresponding nucleotide sequence for which is (SEQ ID NO: 80; CAN-ZIL8_VH), ZIL8 variable light chain (SEQ ID NO: 81; CAN-ZIL8_VL), the corresponding nucleotide sequence for which is (SEQ ID NO: 82; CAN-ZIL8_VL); ZIL9 variable heavy chain (SEQ ID NO:83; CAN-ZIL9_VH), the corresponding nucleotide sequence for which is (SEQ ID NO: 84; CAN-ZIL9_VH), ZIL9 variable light chain (SEQ ID NO: 85; CAN-ZIL9_VL), the corresponding nucleotide sequence for which is (SEQ ID NO: 86; CAN-ZIL9_VL); ZIL111 variable heavy chain (SEQ ID NO:87; CAN-ZIL11_VH), the corresponding nucleotide sequence for which is (SEQ ID NO: 88; CAN-ZIL11_VH), ZIL11 variable light chain (SEQ ID NO: 89; CAN-ZIL11_VL), the corresponding nucleotide sequence for which is (SEQ ID NO: 90; CAN-ZIL11_VL); ZIL69 variable heavy chain (SEQ ID NO:91; CAN-ZIL69_VH), the corresponding nucleotide sequence for which is (SEQ ID NO: 92; CAN-ZIL69_VH), ZIL69 variable light chain (SEQ ID NO: 93; CAN-ZIL69_VL), the corresponding nucleotide sequence for which is (SEQ ID NO: 94; CAN-ZIL69_VL); ZIL94 variable heavy chain (SEQ ID NO:95; CAN-ZIL94_VH), the corresponding nucleotide sequence for which is (SEQ ID NO: 96; CAN-ZIL94_VH), ZIL94 variable light chain (SEQ ID NO: 97; CAN-ZIL94_VL), the corresponding nucleotide sequence for which is (SEQ ID NO: 98; CAN-ZIL94_VL); ZIL154 variable heavy chain (SEQ ID NO:99; CAN-ZIL154_VH), the corresponding nucleotide sequence for which is (SEQ ID NO: 100; CAN-ZIL154_VH), ZIL154 variable light chain (SEQ ID NO: 101; CAN-ZIL154_VL), the corresponding nucleotide sequence for which is (SEQ ID NO: 102; CAN-ZIL154_VL); ZIL159 variable heavy chain (SEQ ID NO:103; CAN-ZIL159_VH), the corresponding nucleotide sequence for which is (SEQ ID NO: 104; CAN-ZIL159_VH), ZIL159 variable light chain (SEQ ID NO: 105; CAN-ZIL159_VL), the corresponding nucleotide sequence for which is (SEQ ID NO: 106; CAN-ZIL159_VL); ZIL171 variable heavy chain (SEQ ID NO:107; CAN-ZIL171_VH), the corresponding nucleotide sequence for which is (SEQ ID NO: 108; CAN-ZIL171_VH), ZIL171 variable light chain (SEQ ID NO: 109; CAN-ZIL171_VL), the corresponding nucleotide sequence for which is (SEQ ID NO: 110; CAN-ZIL171_VL).

The aforementioned nine monoclonal antibodies which were selected for further characterization may be referred to elsewhere in the specification, Figures, or claims as ZIL1, ZIL8, ZIL8, ZIL11, ZIL69, ZIL94, ZIL154, ZIL159, and ZIL171.

### 1.7. Construction of Recombinant Chimeric and Fully Canine Antibodies

Antibody variable domains are responsible for antigen binding. Grafting of the full variable domain onto respective constant region is expected to have little or no impact on the antibody's ability to bind the IL-31 immunogen. To simultaneously confirm that the correct sequence of the heavy and light chain variable regions were identified and to produce homogenous material, expression vectors were designed to produce recombinant chimeric or fully canine antibodies in mammalian expression systems. Chimeric antibodies described here consist of the variable sequence (both CDR and framework) from the host species antibody grafted onto the respective heavy and light constant regions of a feline or canine IgG molecule (for example; mouse variable: canine constant is referred to as mouse:canine chimera). Fully canine antibodies described here consist of the variable sequence (both CDR and framework) from the host species antibody (canine) grafted on to the respective heavy and light chain constant regions of the canine IgG molecule. Synthetic DNA sequences were constructed for the variable heavy (VH) and variable light (VL) sequences of selected antibodies. These sequences contain unique restriction endonuclease sites, a Kozak consensus sequence and, an N-terminal secretion leader to facilitate expression and secretion of the recombinant antibody from a mammalian cell line.

For mouse: feline chimeras, each respective variable region was cloned into a mammalian expression plasmid containing either the feline IgG heavy (SEQ ID NO: 173; Feline_HC_AlleleA_1) the corresponding nucleotide sequence for which is (SEQ ID NO: 174; Feline_HC_AlleleA_1) or light chain (SEQ ID NO: 175; Feline_LC_Kappa_G_minus) the corresponding nucleotide sequence for which is (SEQ ID NO: 176; Feline_LC_Kappa_G_minus) constant regions. For mouse: canine chimeras or fully canine antibodies, each mouse or canine variable region was cloned into a mammalian expression plasmid containing either the canine IgG heavy (SEQ ID NO: 177; Canine_HC_65_1) the corresponding nucleotide sequence for which is (SEQ ID NO: 178; Canine_HC_65_1) or light chain (SEQ ID NO: 179; Canine_LC_Kappa) the corresponding nucleotide sequence for which is (SEQ ID NO: 180; Canine_LC_Kappa) constant regions. The plasmids encoding each heavy and light chain, under the control of the CMV promoter, were co-transfected into HEK 293 cells using standard methods. Following six days of expression, chimeric mAbs were purified from 50 ml of transiently transfected HEK293FS cell supernatants using MabSelect Sure protein A resin (GE Healthcare, Uppsala, Sweden) according to standard methods for protein purification. Eluted fractions were pooled, concentrated to ˜500 μl using a 10,000 nominal MW cutoff Nanosep Omega centrifugal device (Pall Corp., Port Washington, NY), dialyzed overnight at 4° C. in 1×PBS, pH7.2 and stored at 4° C. for further use. Affinity and cell based potency of select recombinant antibodies are described below.

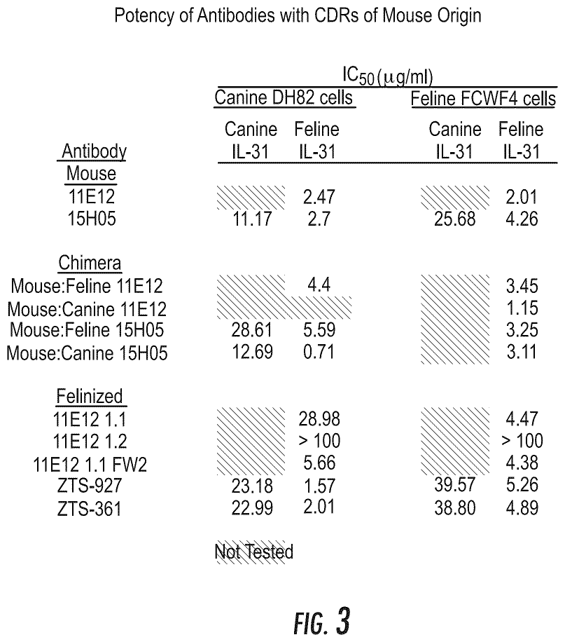

FIG. 2 details the affinity of antibodies with CDRs derived from mouse origin using biacore. FIG. 2, Section A shows the affinity of mouse anti IL-31 antibodies 11E12 and 15H05 and the corresponding affinities of the feline and canine chimeric forms to both feline and canine IL-31 surfaces. These observations confirm the correct sequence for both mouse antibodies and indicate conversion to the chimeric form results in antibodies with equivalent or higher affinity when compared to the mouse parent with the exception of the mouse:feline 15H05 chimera which lost some affinity to both IL-31 species as a result of its conversion to the chimeric form. Fully mouse and chimeric forms of antibodies 11E12 and 15H05 were also tested for activity in the canine and feline cellular assays described in section 1.5. FIG. 3 shows the results for these assays. Mouse antibodies 11E12 and 15H05 were tested for activity against canine and feline cell types using both canine and feline IL-31 to stimulate signaling. The potency of both mouse antibodies was comparable against both canine and feline cells using the feline cytokine with the exception of 15H05 against feline IL-31 in feline FCWF4 cells that shows a slight increase in IC50. Mouse 15H05 was capable of blocking canine IL-31 signaling in both feline and canine cells with the potency in the canine assay being slightly higher. These results indicate that the respective epitopes recognized by these antibodies exists on both canine and feline IL-31 and binding of these antibodies is capable of neutralizing receptor-mediated cellular signaling in a relevant cell line from both species.

FIG. 3 also describes the potency of select chimeras in both cellular assays. Conversion of mouse antibodies to feline and canine chimeras had minimal impact on the potency against feline IL-31 in the feline potency assay (IC50 range 1.15-3.45 μg/ml). Similar results were observed when these chimeras were tested against feline IL-31 signaling on the canine DH82 cell line with a slight increase in potency (IC50=0.71 μg/ml) observed for the 15H05 mouse: canine chimera. In general there was an increase in IC50 values against canine IL-31 in both canine and feline cell types. The mouse: feline 15H05 chimera was slightly less potent in this assay format compared to the mouse: canine form (IC50 28.61 vs. 12.49 μg/ml). Consistent with observations for the mouse antibodies, conversion to canine and feline chimeric forms resulted in minimal changes in potency.

Antibodies described above that were identified from single B cells of immunized dogs were constructed as recombinant IgG proteins following identification of their variable domain sequences. Grafting of these variable domains onto the canine heavy chain Fc (65_1 isotype) resulted in the generation of recombinant fully canine antibodies. It was of interest to identify additional canine antibodies that bound wildtype feline IL-31 and who's binding was decreased to the feline IL-31 15H05 mutant (i.e. are directed at the 15H05 epitope). These antibodies obtained from this alternate source (canine vs. mouse) provide additional paratopes (the portion of the antibody which recognizes the IL-31 protein, includes CDRs) recognizing the 15H05 epitope thus increasing the diversity of antibodies with different physical properties to select from.

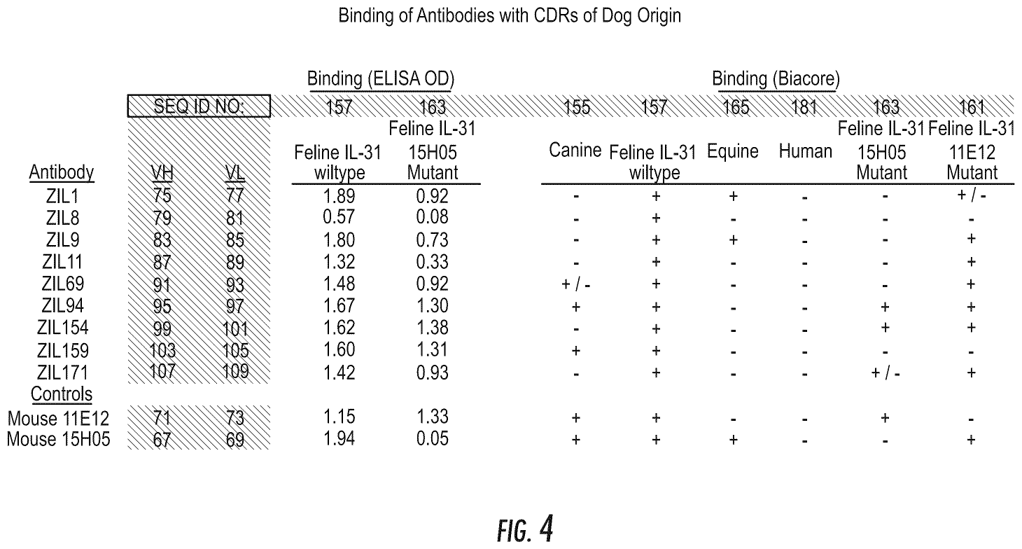

FIG. 4 shows the results obtained for binding of these recombinant canine antibodies to various proteins using both ELISA and Biacore methods. For the indirect ELISA method, antibody binding to wildtype and feline IL-31 15H05 mutant proteins was assessed. All nine canine monoclonal antibodies (ZIL1, ZIL8, ZIL8, ZIL11, ZIL69, ZIL94, ZIL154, ZIL159, and ZIL171) were capable of binding to wildtype feline IL-31 and binding was impacted by mutations in the mAb 15H05 epitope region confirming the correct binding phenotype determined during the initial screening used to identify them. In comparison, the 11E12 antibody bound to the wild-type feline IL-31 and its binding was not impacted by the mutations in the 15H05 epitope region as evidenced by the data in FIG. 4. To confirm binding, biacore analysis was performed using canine, feline, equine, human, feline 15H05 mutant, and feline 11E12 mutant IL-31 proteins as surfaces and a single test concentration of antibody. Similar to ELISA observations, all antibodies tested bound to wildtype feline IL-31. In agreement with the data described above in this section, mouse antibodies 11E12 and 15H05 both bound to canine and feline IL-31 surfaces. Three additional antibodies where shown to have this dual binding property, ZIL69 (partial canine binding), ZIL94, and ZIL159. From this group of nine fully canine antibodies, only ZIL1 and ZIL9 cross-reacted with equine IL-31. Of note, antibody 15H05 was the only one of all assayed herein that bound to canine, feline, and equine IL-31 indicating some level of epitope conservation across the three species. In contrast, none of the antibodies described herein bound to human IL-31. Additional biacore surfaces were used to verify ELISA observations showing differential binding of antibodies to wildtype feline IL-31 and two proteins with mutations in the 15H05 (15H05 mutant) or 11E12 (11E12 mutant) epitopes. As expected, control mouse antibody 11E12 bound to the 15H05 IL-31 mutant and did not bind to the 11E12 IL-31 mutant due to mutations in the epitope. Likewise mouse 15H05 did not bind to the 15H05 mutant and retained binding to the 11E12 IL-31 mutant further distinguishing the separate binding epitopes recognized by these two antibodies. In agreement with the ELISA results, all fully canine antibodies were impacted by the 15H05 mutation with the exception of ZIL94, ZIL154, and ZIL171 (partially affected). Differing results can be attributed to differences in the two assay methodologies. In addition, binding of three antibodies was also shown to be impacted by the 11E12 mutation; ZIL1 (partially effected), ZIL8, and ZIL159. These results indicate the epitope recognized by these antibodies is impacted by changes in both regions of the IL-31 protein. Taken together these results support the characterization nine antibodies derived from canine B cells sharing binding to a region on the feline IL-31 protein that is recognized by antibody 15H05.

### 1.8. Felinization of the Murine 11E12 and 15H05 Antibodies and Optimization of Binding Affinities

The generation of anti-drug antibodies (ADAs) can been associated with loss of efficacy for any biotherapeutic protein including monoclonal antibodies. Comprehensive evaluation of the literature has shown that speciation of monoclonal antibodies can reduce the propensity for mAbs to be immunogenic although examples of immunogenic fully human mAbs and non-immunogenic chimeric mAbs can be found. To help mitigate risks associated with ADA formation for the anti-IL-31 monoclonal antibodies provided herein, a felinization strategy was employed. This felinization strategy is based on identifying the most appropriate feline germline antibody sequence for CDR grafting. Following extensive analysis of all available feline germline sequences for both the variable heavy and light chain, germline candidates were selected based on their homology to the mouse mAbs, and the CDRs from the mouse progenitor mAbs were used to replace native feline CDRs. The objective was to retain high affinity and cell-based activity using feline antibody frameworks to minimize the potential of immunogenicity in vivo. Felinized mAbs were expressed and characterized for their affinity to feline IL-31 and their potency in cell-based assays. In the event that a felinized antibody loses its ability to bind IL-31, a systematic dissection was undertaken to identify; 1) the chain responsible for the loss of function, 2) the framework responsible for the loss of function and 3) the amino acid(s) responsible for the loss function.

Synthetic nucleotide constructs representing the felinized variable heavy and light chains of mAbs 11E12 and 15H05 were made. Following subcloning of each variable chain into plasmids containing the respective feline heavy or kappa constant region, plasmids were co-transfected for antibody expression in HEK 293 cells. Initial attempts at felinization of antibody 11E12 focused on utilization of a single feline VH framework (SEQ ID NO: 111; FEL_11E12_VH1) the corresponding nucleotide sequence for which is (SEQ ID NO: 112; FEL_11E12_VH1) paired independently with VL frameworks (SEQ ID NO: 113; FEL_11E12_VL1) the corresponding nucleotide sequence for which is (SEQ ID NO: 114; FEL_11E12_VL1) and (SEQ ID NO: 115; FEL_11E12_VL2) the corresponding nucleotide sequence for which is (SEQ ID NO: 116; FEL_11E12_VL2) to form Feline 11E12 1.1 and Feline 11E12 1.2 respectively. This attempt at speciation resulted in a loss of affinity with Feline 11E12 1.1 to both the feline and canine IL-31 proteins and a total loss of binding with the Feline 11E12 1.2 mAb when compared to the mouse form of the antibody (FIG. 2, Section B). Potency of these speciated antibodies was tested in the canine DH82 and Feline FCWF4 cell assays using the feline IL-31 cytokine. Felinized 11E12 1.1 had approximately a two-fold decrease in potency against feline IL-31 in the feline FCWF assay when compared to the mouse version of the antibody. In agreement with the loss of affinity for felinized 11E12 1.2, a complete loss of cellular potency was observed for this antibody (FIG. 3). Based on previous experience during caninization of the mAb 11E12 ortholog, a similar strategy was undertaken in attempt to restore the affinity loss to felinization (U.S. Pat. No. 8,790,651 to Bammert, et al.). Substitution of the felinized framework 2 (FW2) region of Feline 11E12 VL1 with the mouse FW2 from (SEQ ID NO: 73; Mu_11E12_VL) the corresponding nucleotide sequence for which is (SEQ ID NO: 74; Mu_11E12_VL) was done to generate Feline 11E12 VL1 FW2. In addition, a single substitution at position 46 of the feline VL (K46Q) was performed to generate (SEQ ID NO: 119; FEL_11E12_VL1_K46Q) the corresponding nucleotide sequence for which is (SEQ ID NO: 120; FEL_11E12_VL1_K46Q). Pairing of the above VLs with Fel_11E12_VH1 resulted in Feline 11E12 1.1 FW2 and Feline 11E12 1.1 K46Q respectively. Changing FW2 resulted in a restoration of affinity for Feline 11E12 1.1 FW2 to the feline IL-31 protein resulting in a KD equivalent to that of the mouse and chimeric form (FIG. 2, Sections A and B). These changes however had a detrimental effect on Feline 11E12 1.1 FW2s affinity to the canine IL-31 protein indicating a clear distinction in the nature of antibody 11E12s ability to bind this epitope on the feline and canine cytokine. The single amino acid substitution in Feline 11E12 1.1 K46Q was unable to influence affinity of this antibody. Increased affinity of antibody 11E12 1.1 FW2 for the feline IL-31 protein resulted in increased potency against the feline cytokine in the canine DH82 assay (FIG. 3).

Felinization efforts with mouse antibody 15H05 focused on the combinations of three feline VH frameworks with three feline VL frameworks for a total of 9 felinized mAbs. FEL_15H05_VH1 (SEQ ID NO: 121; FEL_15H05_VH1), the corresponding nucleotide sequence for which is (SEQ ID NO: 122; FEL_15H05_VH1) was combined with (SEQ ID NO: 127; FEL_15H05_VL1), the corresponding nucleotide sequence for which is (SEQ ID NO: 128; FEL_15H05_VL1), (SEQ ID NO: 129; FEL_15H05_VL2), the corresponding nucleotide sequence for which is (SEQ ID NO: 130; FEL_15H05_VL2), and (SEQ ID NO: 131; FEL_15H05_VL3), the corresponding nucleotide sequence for which is (SEQ ID NO: 132; FEL_15H05_VL3) were used to create Feline 15H05 1.1, Feline 15H05 1.2, and Feline 15H05 1.3 respectively. FEL_15H05_VH2 (SEQ ID NO: 123; FEL_15H05_VH2), the corresponding nucleotide sequence for which is (SEQ ID NO: 124; FEL_15H05_VH2) was combined with (SEQ ID NO: 127; FEL_15H05_VL1), the corresponding nucleotide sequence for which is (SEQ ID NO: 128; FEL_15H05_VL1), (SEQ ID NO: 129; FEL_15H05_VL2), the corresponding nucleotide sequence for which is (SEQ ID NO: 130; FEL_15H05_VL2), and (SEQ ID NO: 131; FEL_15H05_VL3), the corresponding nucleotide sequence for which is (SEQ ID NO: 132; FEL_15H05_VL3) were used to create Feline 15H05 2.1, Feline 15H05 2.2, and Feline 15H05 2.3 respectively. FEL_15H05_VH3 (SEQ ID NO: 125; FEL_15H05_VH3), the corresponding nucleotide sequence for which is (SEQ ID NO: 126; FEL_15H05_VH3) was combined with (SEQ ID NO: 127; FEL_15H05_VL1), the corresponding nucleotide sequence for which is (SEQ ID NO: 128; FEL_15H05_VL1), (SEQ ID NO: 129; FEL_15H05_VL2), the corresponding nucleotide sequence for which is (SEQ ID NO: 130; FEL_15H05_VL2), and (SEQ ID NO: 131; FEL_15H05_VL3), the corresponding nucleotide sequence for which is (SEQ ID NO: 132; FEL_15H05_VL3) were used to create Feline 15H05 3.1, Feline 15H05 3.2, and Feline 15H05 3.3 respectively. Similar to observations with antibody 11E12, the first attempt at felinization of antibody 15H05 resulted in a loss of affinity to the feline IL-31 protein when compared to mouse 15H05 and a neutral affect when compared to the 15H05 mouse feline chimera (FIG. 2, Sections A and C). Similar to observations with felinized antibody 11E12 binding to canine IL-31, certain combinations of feline 15H05 VH and VL frameworks had a neutral to positive impact on affinity to canine IL-31 (See FIG. 2, Section C: Feline 15H05 1.1, 2.2, and 3.2).

In an effort to restore the affinity of felinized antibody 15H05, each felinized 15H05 VH was paired with the mouse 15H05 VL to generate heterochimeric antibodies. FEL_15H05_VH1 (SEQ ID NO: 121; FEL_15H05_VH1), the corresponding nucleotide sequence for which is (SEQ ID NO: 122; FEL_15H05_VH1) was combined with MU_15H05_VL (SEQ ID NO: 69; MU_15H05_VL), the corresponding nucleotide sequence for which is (SEQ ID NO: 70; MU_15H05_VL) to generate Feline 15H05 VH1 mouse VL. FEL_15H05_VH2 (SEQ ID NO: 123; FEL_15H05_VH2), the corresponding nucleotide sequence for which is (SEQ ID NO: 124; FEL_15H05_VH2) was combined with MU_15H05_VL (SEQ ID NO: 69; MU_15H05_VL), the corresponding nucleotide sequence for which is (SEQ ID NO: 70; MU_15H05_VL) to generate Feline 15H05 VH2 mouse VL. FEL_15H05_VH3 (SEQ ID NO: 125; FEL_15H05_VH3), the corresponding nucleotide sequence for which is (SEQ ID NO: 126; FEL_15H05_VH3) was combined with MU_15H05_VL (SEQ ID NO: 69; MU_15H05_VL), the corresponding nucleotide sequence for which is (SEQ ID NO: 70; MU_15H05_VL) to generate Feline 15H05 VH3 mouse VL. These felinized VH mouse VL heterochimeras were analyzed for their affinity to canine and feline IL-31. Pairing of felinized 15H05 VH1 and VH3 with mouse 15H05 VL restored the affinity to feline IL-31 to equivalent or better than the mouse and chimeric forms. This trend in improved affinity was also observed to the canine IL-31 protein (FIG. 2, Sections A and C).

To further dissect the positions in the 15H05 frameworks responsible for affinity loss, a single felinized VH of 15H05 (FEL_15H05_VH1) was used to pair with individual framework substitutions from mouse 15H05 VL. FEL_15H05_VH1 (SEQ ID NO: 122; FEL_15H05_VH1), the corresponding nucleotide sequence for which is (SEQ ID NO: 123; FEL_15H05_VH1) was combined independently with FEL_15H05_VL1_FW1 (SEQ ID NO: 133; FEL_15H05_VL1_FW1), the corresponding nucleotide sequence for which is (SEQ ID NO: 134; FEL_15H05_VL1_FW1), FEL_15H05_VL1_FW2 (SEQ ID NO: 135; FEL_15H05_VL1_FW2), the corresponding nucleotide sequence for which is (SEQ ID NO: 136; FEL_15H05_VL1_FW2), and FEL_15H05_VL1_FW3 (SEQ ID NO: 137; FEL_15H05_VL1_FW3), the corresponding nucleotide sequence for which is (SEQ ID NO: 138; FEL_15H05_VL1_FW3) to create Feline 15H05 1.1 FW1, Feline 15H05 1.1 FW2, and Feline 15H05 1.1 FW3 respectively. Substitution of mouse 15H05 FW1 onto Feline 15H05 1.1 was detrimental to the affinity to both feline and canine IL-31, however, when mouse FW2 or FW3 were substituted on Feline 15H05 1.1, excellent affinity was achieved to canine and feline IL-31 with the FW2 being superior for both species (FIG. 2, Section C). Additional pairwise framework substitutions were performed to determine the extent of affinity modulation by this approach. FEL_15H05_VH1 (SEQ ID NO: 121; FEL_15H05_VH1), the corresponding nucleotide sequence for which is (SEQ ID NO: 122; FEL_15H05_VH1) was combined independently with FEL_15H05_VL1_FW1_2 (SEQ ID NO: 139; FEL_15H05_VL1_FW1_FW2), the corresponding nucleotide sequence for which is (SEQ ID NO: 140; FEL_15H05_VL1_FW1_FW2), FEL_15H05_VL1_FW2_3 (SEQ ID NO: 143; FEL_15H05_VL1_FW2_FW3), the corresponding nucleotide sequence for which is (SEQ ID NO: 144; FEL_15H05_VL1_FW2_FW3), and FEL_15H05_VL1_FW1_3 (SEQ ID NO: 141; FEL_15H05_VL1_FW1_FW3), the corresponding nucleotide sequence for which is (SEQ ID NO: 142; FEL_15H05_VL1_FW1_FW3) to give Feline 15H05 1.1 FW1_2, Feline 15H05 1.1 FW2_3, and Feline 15H05 1.1 FW13 respectively. Interestingly, the substitution of mouse FW1 alone was detrimental to affinity while combinations of FW1 with FW2 or FW3 resulted in good affinity to both feline and canine IL-31 (FIG. 2, Section C).

Finally, an attempt was made to minimize the number of backmutations in the feline frameworks beginning with the most promising combinations of felinized VH and VL sequences. For this, FEL_15H05_VH1 (SEQ ID NO: 121; FEL_15H05_VH1), the corresponding nucleotide sequence for which is (SEQ ID NO: 122; FEL_15H05_VH1) was combined independently with FEL_15H05_VL1_FW2_K42N (SEQ ID NO: 145; FEL_15H05_VL1_FW2_K42N), the corresponding nucleotide sequence for which is (SEQ ID NO: 146; FEL_15H05_VL1_FW2_K42N), FEL_15H05_VL1_FW2_V43I (SEQ ID NO: 147; FEL_15H05_VL1_FW2_V43I), the corresponding nucleotide sequence for which is (SEQ ID NO: 148; FEL_15H05_VL1_FW2_V43I), FEL_15H05_VL1_FW2_L46V (SEQ ID NO: 149; FEL_15H05_VL1_FW2_L46V), the corresponding nucleotide sequence for which is (SEQ ID NO: 150; FEL_15H05_VL1_FW2_L46V), FEL_15H05_VL1_FW2_Y49N (SEQ ID NO: 151; FEL_15H05_VL1_FW2_Y49N), the corresponding nucleotide sequence for which is (SEQ ID NO: 152; FEL_15H05_VL1_FW2_Y49N), and FEL_15H05_VL1_FW2_K42N_V43I (SEQ ID NO: 153; FEL_15H05_VL1_FW2_K42N_V43I), the corresponding nucleotide sequence for which is (SEQ ID NO: 154; FEL_15H05_VL1_FW2_K42N_V43I) to give Feline 15H05 1.1 K42N, Feline 15H05 1.1 V43I, Feline 15H05 1.1 L46V, Feline 15H05 1.1 Y49N, and Feline 15H05 1.1 K42N_V43I respectively. While the substitution of the entire mouse FW2 framework onto Felinized 15H05 VL1 resulted in an antibody with excellent affinity to canine and feline IL-31 (FIG. 2, Section C, Feline 15H05 1.1 FW2), the individual backmutations of FW2 amino acid residues had a neutral or detrimental effect indicating all 4 substitutions are necessary to maintain the optimal tertiary structure for positioning of the CDRs on the IL-31 epitope.

Increased affinity of felinized 15H05 1.1 FW2 to feline and canine IL-31 lead to the selection of this antibody for further work.

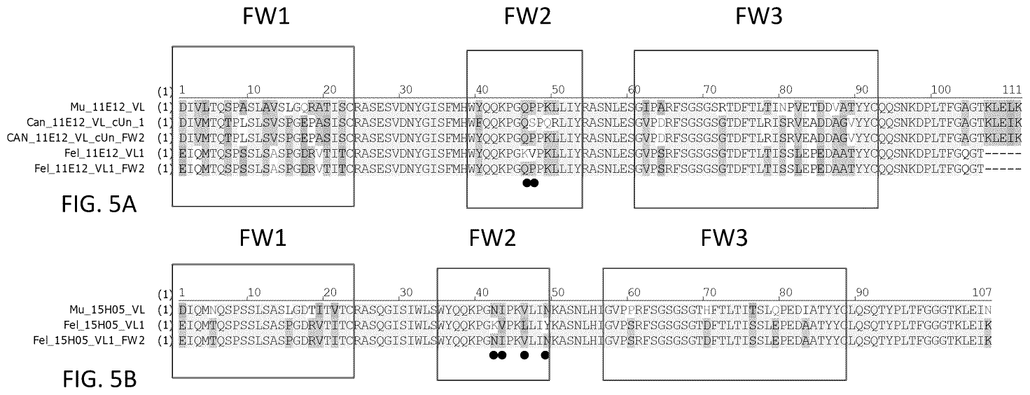

FIG. 5A shows an alignment of mouse antibody 11E12 VL sequence comparing previously referenced caninized 11E12 sequence to the felinized versions. Noted below the alignment are dots showing the positons of relevant changes to Fel_11E12_VL1 that were necessary to restore affinity of this antibody to the IL-31 protein. Likewise FIG. 5B shows the necessary changes to the felinized 15H05 VL (Fel_15H05_VL1) that were required to not only restore, but improve, its affinity to canine and feline IL-31 when compared to the mouse and chimeric forms of this antibody.

1.9. Generation of Cell Lines Expressing Felinized Anti IL-31 Antibodies from Glutamine Synthetase (GS) Plasmids

Felinized 15H05 1.1 FW2 was chosen as a candidate for the generation of stable cell lines that will produce a homogenous supply of the antibody for further characterization. The genes encoding the felinized heavy and light chains for cell line production were cloned into GS plasmids pEE 6.4 and pEE 12.4 respectively (Lonza, Basel, Switzerland). The resulting plasmids were digested according to the manufacturer's protocol and ligated together to form a single mammalian expression plasmid. For ZTS-927, the heavy chain is (SEQ ID NO: 121; FEL_15H05_VH1), the corresponding nucleotide sequence for which is (SEQ ID NO: 122; FEL_15H05_VH1) combined with feline IgG heavy chain constant (SEQ ID NO: 171; Feline_HC_AlleleA_wt), the corresponding nucleotide sequence for which is (SEQ ID NO: 172; Feline_HC_AlleleA_wt). For ZTS-927, the light chain is (SEQ ID NO: 135; FEL-15H05-VL1_FW2), the corresponding nucleotide sequence for which is (SEQ ID NO: 136; FEL-15H05-VL1_FW2) combined with feline IgG light chain constant (SEQ ID NO: 175; Feline_LC_Kappa_G_minus), the corresponding nucleotide sequence for which is (SEQ ID NO: 176; Feline_LC_Kappa_G_minus). For ZTS-361, the heavy chain is (SEQ ID NO: 121; FEL_15H05_VH1), the corresponding nucleotide sequence for which is (SEQ ID NO: 122; FEL_15H05_VH1) combined with feline IgG heavy chain constant (SEQ ID NO: 173; Feline_HC_AlleleA_1), the corresponding nucleotide sequence for which is (SEQ ID NO: 174; Feline_HC_AlleleA_1). For ZTS-361, the light chain is (SEQ ID NO: 135; FEL-15H05-VL1_FW2), the corresponding nucleotide sequence for which is (SEQ ID NO: 136; FEL-15H05-VL1_FW2) combined with feline IgG light chain constant (SEQ ID NO: 175; Feline_LC_Kappa_G_minus), the corresponding nucleotide sequence for which is (SEQ ID NO: 176; Feline_LC_Kappa_G_minus). For ZTS-1505, the heavy chain is (SEQ ID NO: 121; FEL_15H05_VH1), the corresponding nucleotide sequence for which is (SEQ ID NO: 122; FEL_15H05_VH1) combined with feline IgG heavy chain constant (SEQ ID NO: 173; Feline_HC_AlleleA_1), the corresponding nucleotide sequence for which is (SEQ ID NO: 174; Feline_HC_AlleleA_1). For ZTS-1505, the light chain is (SEQ ID NO: 135; FEL-15H05-VL1_FW2), the corresponding nucleotide sequence for which is (SEQ ID NO: 136; FEL-15H05-VL1_FW2) combined with feline IgG light chain constant (SEQ ID NO: 186; Feline_LC_Kappa_G_minus_QRE_minus), the corresponding nucleotide sequence for which is (SEQ ID NO: 187; Feline_LC_Kappa_G_minus_QRE_minus). The affinity and potency data for ZTS-1505 is described below in section 1.18 of this example section.

To demonstrate transient production of antibody, each plasmid was used to transfect HEK 293 cells and expression was carried out in various size cultures. Protein was isolated from conditioned HEK medium using Protein A affinity chromatography according to standard protein purification methods. Medium was loaded onto chromatographic resin and eluted by pH shift. Eluted protein was pH adjusted, dialyzed, and sterile filtered prior to use. ZTS-361 was subsequently used for evaluation in the cat pruritus model to evaluate in vivo efficacy. Antibodies produced from a single GS plasmid, ZTS-927 and ZTS-361, were tested for affinity and potency. FIG. 2, Section D shows the results for the affinity assessment of these antibodies using biacore. The affinity of ZTS-927 and ZTS-361 to feline IL-31 is highly consistent with that of the mouse and chimeric form of the progenitor mouse mAb 15H05. The potency of these two antibodies was determined against canine and feline IL-31 using both canine and feline cell assays (FIG. 3). Consistent with previous observations the IC50 values were proportionally higher when using the canine form of IL-31 with both cell types. The IC50 values for ZTS-927 and ZTS-361 against feline IL-31 were also highly consistent with values derived from the chimeric and mouse form of the antibody indicating the final felinized version of mAb 15H05 produced form a single GS plasmid was suitable for cell line development.

For generation of a stable cell line producing candidate antibodies, the GS plasmid was linearized prior to transfection with the restriction enzyme, Pvul, which cuts at a single site in the plasmid backbone. GS-CHOK1SV (clone 144E12) cells were transfected with linearized plasmid DNA via electroporation. Following transfection, cells were plated in 48-well plates (48WP) in order to generate stable pools. When pools were at least 50% confluent in the 48WPs, 100 μl of supernatant was analyzed for IgG expression using the ForteBio Octet and protein A biosensors (Pall ForteBio, Fremont, CA). The best expressing clones were scaled up into 6 well-plates (6 WP) and then into 125 mL shake flasks (SF). Once cells adapted to suspension culture in 125 mL flasks, 2 vials of each cell line pool were banked for LN storage. Since manufacturing cell lines must be clonal, the top 3 highest expressing pools were subcloned by limiting dilution in 96 well culture plates. In order to prove clonality and avoid a second round of limiting dilution, 96 well plates were imaged using Molecular Devices Clone-Select Imager (CSI) (Molecular Devices LLC, San Jose, CA) which captures images of single-cells and their subsequent growth. Clones were selected based on successful CSI images, growth and production in 96WPs.

In order to assess cell culture growth and productivity, the top expressing pools were further evaluated in a 14-day fed batch in 125 mL SFs. Cells were seeded in platform media and feeds consisting of Life Technologies' CD CHO plus 4 amino acids, proprietary feed CDF v6.2, and 10% glucose. Following the 14-day Fed-Batch, pools were centrifuged and the CD CHO produced mAB was isolated by filtering the supernatant via a 0.20 μm Polyethersulfone (PES) membrane prior to purification.

A typical purification consists of two liters of conditioned medium (from CHO cell culture, 0.2 μm filtered) loaded onto a 235 mL column of MabSelect (GE healthcare, cat #17-5199-02). The column had been pre-equilibrated with PBS. The sample was loaded at a residence time of >2.5 minutes. Following load, the column was washed again with PBS, and then with 25 mM sodium acetate, pH ˜neutral. The column was eluted with 25 mM acetic acid, pH 3.6, and then stripped with 250 mM acetic acid, 250 mM sodium chloride, pH ˜2.2. Fractions (50 mL) were collected during the elution and strip steps. UV absorbance at A280 was monitored throughout. Peak fractions were pooled, pH adjusted to ˜5.5 with the addition of 20 mM sodium acetate, and then dialyzed against three exchanges of buffer. The dialysate was collected, sterile filtered, and stored at 4° C.

### 1.10. Identification of the Epitope on IL-31 Recognized by Antibody 15H05

Knowledge of the epitope on IL-31 that is recognized by an antibody is critical to understanding the mechanism by which it neutralizes the cytokine from binding to the IL-31Ra: OSMR co-receptor. In addition, knowing the epitope enables (but is not limited to) optimization of antibody binding affinity and design of peptide epitope mimetics (mimotopes) which can have great utility as analytical capture reagents and as a subunit vaccines to elicit a relevant focused immune response. A multistep process using CLIPS (Chemical Linkage of Peptides onto Scaffolds) technology (Timmerman et al. J Mol Recognit. 2007; 20(5): 283-299) was used to identify and optimize a peptide capable of binding to the paratope of mAb 15H05 (Pepscan, Lelystad Netherlands). The affinity of mAb 15H05 to both canine and feline IL-31 proteins is high (FIG. 2A, MU-15H05) so the primary sequence of both IL-31 species was considered relevant to this effort. A peptide microarray library representing the canine IL-31 protein was created and used to identify peptides capable of binding mAb 15H05 using an indirect ELISA. Following identification of peptides whose primary amino acid sequences represent the binding region of mAb 15H05 on IL-31, a focused full replacement analysis was performed using peptides representing a segment of IL-31 and replacing each of the 12 amino acids in this mAb 15H05 binding region with the 19 other possible amino acid residues at each position. This analysis was essential to identify the key amino acid residues on IL-31 involved with mAb 15H05 binding and also demonstrated where substitutions on the canine primary sequence lead to an enhancement of antibody binding.

The amino acids on the canine IL-31 protein that are recognized by antibody 11E12 were described previously (U.S. Pat. No. 8,790,651 to Bammert, et al.). Therein were described the mutational analysis of the canine IL-31 protein showing positions on the canine IL-31 protein that affect binding of mAb 11E12 when converted to alanine. Based on the full replacement analysis described for mAb 15H05 above and previous knowledge of the binding epitope of 11E12, mutant forms of the feline IL-31 protein were created by substituting alanine for two key residues on the epitope recognized by each antibody (mutants described in section 1.2 above). Mutations for each epitope were named according to the antibody that recognizes the site of the mutation (mutant 11E12 and 15H05 vs. the native wt protein sequence).

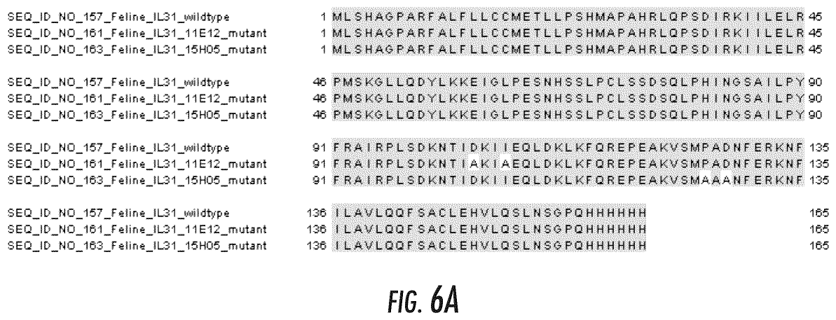

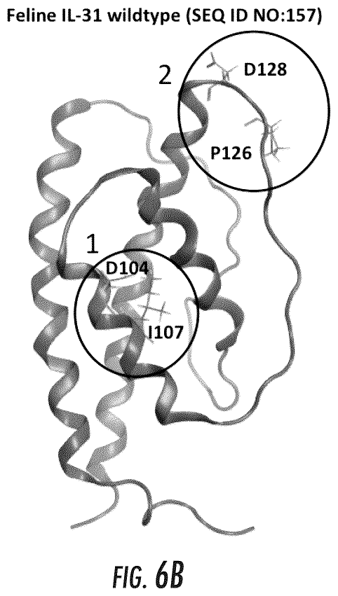

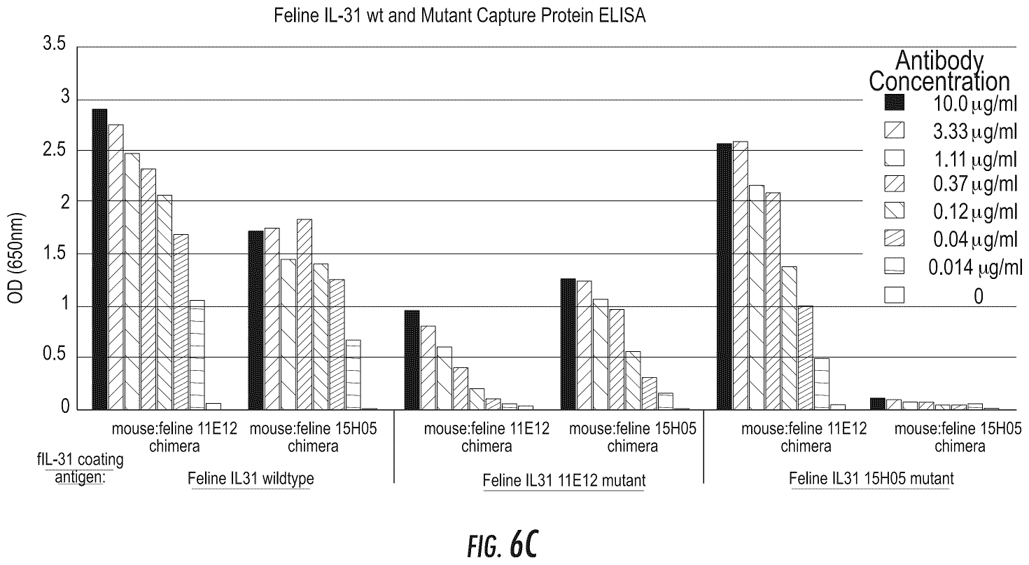

FIG. 6A shows the alignment of wildtype feline IL-31 (SEQ ID NO: 157) with mutants 15H05 (SEQ ID NO: 163) and 11E12 (SEQ ID NO: 161) highlighting the positions where the alanine substitutions occur. IL-31 belongs to the IL-6 family of cytokines with the four helical bundles possessing an up and down architecture (CATH database, Dawson et al. 2017 Nucleic Acids Res. 2017 Jan. 4; 45 (Database issue): D289-D295). A homology model was generated based on the human IL-6 structure 1P9M (Boulanger et al. 2003 Science. June 27; 300(5628):2101-4) using the MOE software (Chemical Computing Group, Montreal, QC, Canada). FIG. 6B shows the feline IL-31 homology model highlighting the positions of the amino acids involved with binding of antibodies 11E12 (site 1) and 15H05 (site 2). The binding sites for each antibody appear to be located at separate positions on the IL-31 protein.

To determine the impact of mAbs 11E12 and 15H05 ability to bind these mutant forms of feline IL-31, an indirect ELISA was run using the mutants directly coated on to an immunoassay plate. FIG. 6C shows the results for this ELISA demonstrating that mAbs 11E12 and 15H05 are capable of binding to wiltype feline IL-31 in this assay format. When mutant 11E12 is used as a capture protein, binding of mAb 11E12 is highly attenuated and binding of mAb 15H05 is partially attenuated. Previous analysis of the 11E12 epitope on canine IL-31 (described in U.S. Pat. No. 8,790,651 to Bammert et al.) indicated that 4 amino acid residues impact binding of the mAb when mutated to alanine so the mutation of 2 residues, in this case, may not be enough to completely eliminate the high affinity binding of mAb 11E12 using this ELISA format. The minor attenuation of mAb 15H05s binding to the 11E12 mutant is likely due to translational effects of the mutations from movement of the two front helices effecting the 15H05 binding site. The mutations designed to affect mAb 15H05s binding (mutant 15H05) show a complete loss of mAb 1505s ability to bind this IL-31 mutant by ELISA. Unlike the 11E12 mutant, the changes in the random coil recognized by mAb 15H05 (mutant 15H05) had no impact on mAb 11E12 binding further supporting the distinction between the two epitopes (FIG. 6C).

1.11. Competition Binding Assessment of mAbs 15H05 and 11E12 Using Biacore

To further characterize the IL-31 epitopes bound by mAbs 15H05 and 11E12, blocking experiments were performed using biacore where the surface containing the IL-31 protein was generated followed by sequential addition of antibodies. FIG. 7A and FIG. 7B show the relative binding of each antibody to IL-31 following capture of 11E12 or 15H05. The columns labeled HBS-EP (assay buffer) indicate the maximum signal obtained from each antibody binding to the IL-31 surface alone without competition. FIG. 7A shows the competition binding data for mouse 15H05 and 11E12 antibodies to canine IL-31. These results clearly indicate that antibodies 15H05 and 11E12 are capable of binding to canine IL-31 in the presence of one another indicating they recognized distinct epitopes on the protein. The sensorgrams related to FIG. 7A show the disassociation kinetics of both antibodies are very slow on this newly formed biacore surface therefore no additional occupation of binding sites can occur with addition of the same antibody (data not shown).

FIG. 7B shows the competition binding data for antibodies 15H05 and 11E12 on a feline IL-31 surface again showing no overlap in the epitope recognized. Binding of additional antibody in the presence of the same antibody is a result of the increased off rate due to the poorer quality of the surface used. Increased off rates can be seen and compared to the KD values from newly formed feline IL-31 surfaces in FIG. 2.

These results further support the epitope mapping data in section 1.10 indicating a distinct epitope is recognized by the CDRs contained in antibody MU-15H05 when compared to MU-11E12. The epitope recognized by antibody 15H05 is distinct from antibody 11E12 described in (U.S. Pat. No. 8,790,651 to Bammert, et al.) and is a novel target on the IL-31 protein for neutralization of this cytokines activity in multiple species. These findings highlight the distinct spatial relationship of binding sites described in the feline IL-31 homology model (FIG. 6B) and support the hypothesis that this face of the cytokine is critical for interaction with the IL-31Ra:OSMR receptor complex.

### 1.12. Synthesis and Characterization of Soluble Feline IL-31 Co-Receptor (IL-31RA and OSMR)

The human IL-31 heteromeric receptor co-complex, consisting of IL31Ra and OSMR subunits, was shown to be required for IL31-mediated intercellular activation of the JAK-STAT pathway and having involvement in atopic skin disease (Dillon et al. 2004 Nat Immunol. July; 5(7):752-60, Dreuw et al. 2004 J Biol Chem. 279:36112-36120; and Diveu et al. 2004 Eur Cytokine Netw. 15:291-302). The human IL-31Ra subunit was later described as the initial binding event that occurs when IL-31 is in contact with cell surface receptors and this event is a pre-requisite for the recruitment of OSMR with subsequent formation of a high affinity co-receptor complex (Le Saux et al. 2010 J Biol Chem. January 29; 285(5):3470-7). We describe here evidence that the feline IL-31 protein is capable of binding to both OSMR and the IL-31Ra independently. This observation is novel and has important implications to understanding how the IL-31 protein interacts with the IL-31Ra:OSMR co-receptor and to the biological role of IL-31 as it interacts independently with individual subunits.

To enable understanding of how IL-31 binds to its co-receptor and to characterize the inhibitory properties of identified antibodies, two receptor forms were synthesized. The individual IL-31 receptor subunit IL-31Ra (SEQ ID NO: 169; Feline_IL_31Ra_HIgG1_Fc_X1_Fn3) the corresponding nucleotide sequence for which is (SEQ ID NO: 170; Feline_IL_31Ra_HIgG1_Fc_X1_Fn3), and OSMR- (SEQ ID NO: 167; Feline_OSMR_hIgG1_Fc) the corresponding nucleotide sequence for which is (SEQ ID NO: 168; Feline_OSMR_hIgG1_Fc) were both constructed as human IgG1 Fc fusions. By homology to the human homologs, the cytokine binding, fibronectin Ill, and Ig-like domains were identified. To evaluate the individual receptor subunits, the extracellular domains of OSMR and the IL-31Ra (with its expected N-terminal proximal fibronectin Ill domain) were generated as human IgG1 Fc fusions, both employing their native signal peptides. All synthetic cassettes were cloned into pcDNA3.1, expressed in the ExpiCHO system and purified as described above.

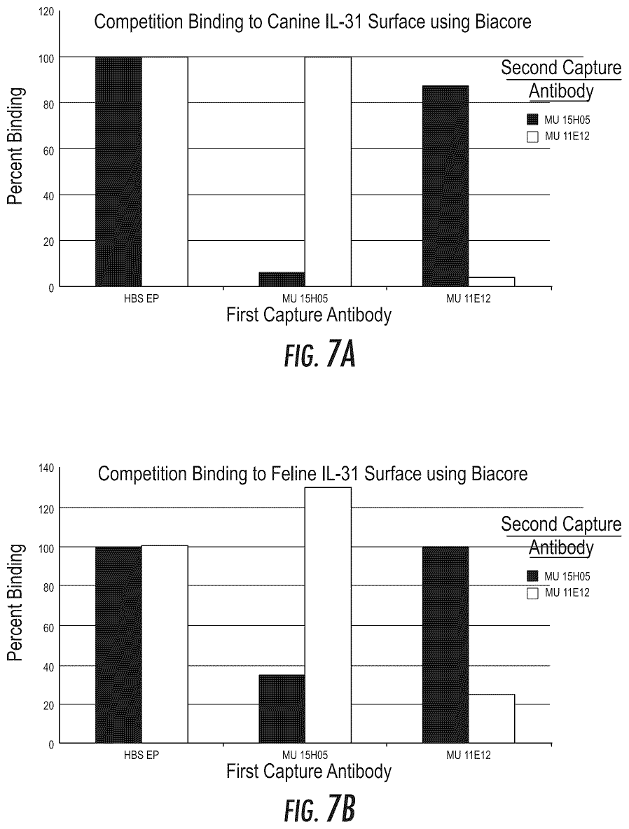

To analyze the ability of these receptor forms to bind wildtype and mutant IL-31 proteins, and indirect ELISA was run by coating 100 ul of each respective protein on an immulon 2HB plate (1 μg/ml) overnight in carb/bicarb buffer (sigma C3041-100CAP) at 4 C. The ELISA plates were then blocked with 5% NFDM blocking buffer in PBST for 1 hour at room temperature followed by binding of multiple concentrations of each receptor construct at room for 1 hour. Following washing with PBST, the presence of the bound receptor (Fc fusion) was identified using mouse anti-human IgG1 (Lifetech A10684, 1:500 dilution) for 1 hour at room temperature. The wells were again washed with PBST and developed with KPL sureblue 3,3′,5,5′-tetra-methylbenzidine (TMB) microwell substrate. FIG. 8 shows the results for this indirect ELISA using wildtype and mutant forms of the feline IL-31 protein as a capture. These data demonstrate the ability of the wildtype feline IL-31 to independently bind to the IL-31Ra and OSMR receptor subunits. These observations are in contrast to previous reports indicating the IL-31 protein initially binds to the IL-31Ra subunit and further recruits OSMR to the site. As the biological role of IL-31 is still being determined, it is of great importance to understand the dynamics of receptor binding and the potential consequences to attenuation of its role in diseases such as atopic dermatitis. For this reason, consideration was further given to these observations when characterizing antibodies the bind to epitopes capable of disrupting the ability of IL-31 to recognize IL-31Ra and OSMR.

In section 1.2 we describe the attenuated binding of antibodies 11E12 and 15H05 to mutants with key amino acids in their binding sites converted to alanine (mutant 11E12 and 15H05 respectively). It was therefore of great interest to understand the impact of these mutations on the ability to bind to the individual IL-31Ra and OSMR receptor subunits. FIG. 8 shows that mutation in either the 11E12 or 15H05 binding site completely disrupts IL-31Ra and OSMRs ability to bind indicating both antibodies bind epitopes that are necessary for interaction of IL-31 with both receptor subunits. Lack of binding could also be due to changes in the confirmation of IL-31 resulting from mutation however these mutants are still capable of binding to antibody which suggests this is not the case. This key finding supports the ability of both antibodies 11E12 and 15H05 (and derivatives) recognizing epitopes on IL-31 that neutralize the cytokines ability to signal through its co-receptor and further block cell association of the cytokine to either receptor during this process. These data support the identification of antibodies that are capable of removing IL-31 from circulation and rendering it unable to bind to cell surface or soluble receptor forms.

### 1.13. In Vivo Evaluation of Chimeric Antibodies in a Feline IL-31 Pruritus Challenge Model

The ability of an antibody to effectively neutralize its target can be assessed in vitro through examination of binding to a relevant epitope on the target protein with the appropriate affinity and potency in a cell based assays that allow extrapolation to in vivo potency. Described above are the steps taken to characterize two series of antibodies generated from the mouse progenitor mAbs 11E12 and 15H05. Section 1.7 describes the generation of mouse: feline chimeric forms of mAbs 11E12 and 15H05 with a resulting affinity to canine and feline IL-31 that are comparable to the original mouse monoclonal antibody (FIG. 2, Section A). The mouse: feline chimeric forms of 11E12 and 15H05 also had comparable IC50 values showing inhibition of feline IL-31 induced pSTAT3 signaling in canine and feline macrophage cells (FIG. 3). During the felinization process in section 1.8, mouse mAb 11E12 was converted to the felinized version (Feline 11E12 1.1) with subsequent loss of affinity to canine and feline IL-31 (FIG. 3) and loss of potency against feline IL-31 signaling in canine and feline cells (FIG. 3). Prior to optimization of the felinized 11E12 and 15H05 antibodies described in section 1.8, it was of interest to understand the ability of these preliminary felinized and chimeric forms to neutralize the pruritic activity of feline IL-31 in a cat challenge model. Of interest was the pharmacodynamic effect of these different antibodies on neutralization of pruritus and to understand any correlation to affinity, cellular potency, or epitope recognition that may influence efficacy. Going forward a range of cellular potency that correlates to in vivo efficacy in the pruritus challenge model could be predictive of further optimization necessary using in vitro assays.

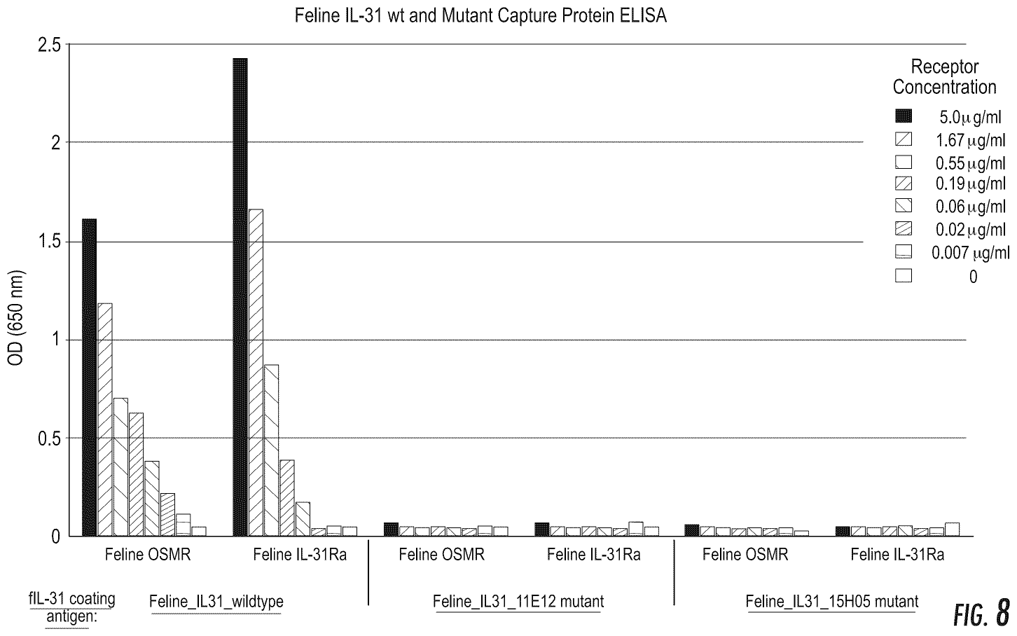

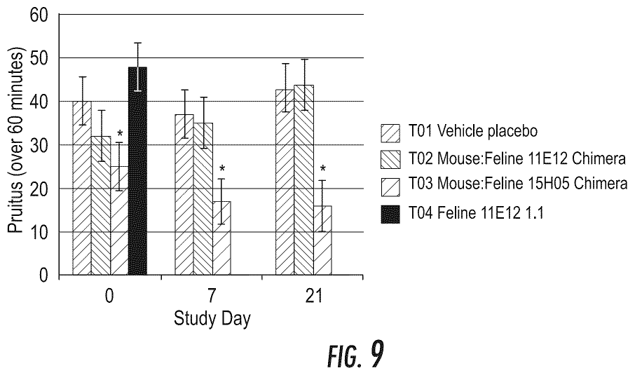

To test the preliminary efficacy of mouse: feline 11E12 chimera, mouse: feline 15H05 chimera, and felinized 11E12 (Feline 11E12 1.1), an IL-31 induced pruritus model in cats was developed. Following an intravenous dose of 0.5 μg/kg feline IL-31 (SEQ ID NO: 159; Feline_IL-31_E_coli), the corresponding nucleotide sequence for which is (SEQ ID NO: 160; Feline_IL-31_E_coli), cats will portray transient pruritic behavior that includes (but is not limited to) licking, chewing, scratching, and head or body shaking. Rubbing up against the cage was not considered a pruritic activity. Pruritic observations take place by a trained investigator for 30 minutes prior to administration of the IL-31 protein and for 1 hour following. For this study, a baseline challenge with feline IL-31 was performed up to 1 month prior to dosing with antibody. On day zero, a 0.5 mg/kg antibody dose was combined with 0.5 μg/kg of feline IL-31 at room temperature for 60 minutes prior to injecting the pre-bound complex into each animal. A “no mAb” control was included for a control. The dose of mAb represents a gross molar excess of antibody to cytokine. Pruritic activity was monitored as described on days 0, 7, and 21. Results in FIG. 9 show significant improvement (p<0.05) in pruritus scores with mAb mouse: feline 15H05 chimera at days 0, 7, and 21 when compared to the placebo control. Although the mouse: feline 11E12 chimera showed an initial trend in efficacy at day zero, it did not achieve a significant reduction in pruritus at any timepoint when compared to vehicle placebo. Feline 11E12 1.1 did not reduce pruritus at day zero and showed no trend in efficacy when compared to vehicle placebo so further IL-31 challenges on days 7 and 21 were not performed.

Taken together these results show a clear delineation between the activities of these antibodies with the lack of efficacy for feline 11E12 1.1 at preventing pruritic behavior in the cat induced by IL-31. The loss of affinity and potency of feline 11E12 1.1 likely resulted in the lack of in vivo efficacy. When comparing the efficacy outcome of mouse: feline 11E12 chimera and mouse: feline 15H05 chimera the distinction is more subtle. The chimeric forms of both mAbs have a comparable KD value to their mouse progenitor with the affinity of mouse: feline 11E12 being slightly superior to both feline and canine IL-31 (FIG. 2, Section A). This increased affinity however does not translate directly to increased potency as the mouse: feline 15H05 chimera has an approximately 2-fold increased IC50 to that of mouse: feline 11E12 chimera against feline IL-31 induced pSTAT3 signaling in feline FCWF4 cells (FIG. 3). These data suggest that the manner in which antibody 15H05 CDRs recognize feline IL-31 is superior at neutralizing the cytokines ability to signal through its co-receptor in turn making it more effective at blocking pruritus in cats. The differences in IC50s observed in these cellular assays offers a promising means to predict in vivo potency and to discriminate subtle differences in epitope recognition both within and between series of antibodies.

### 1.14. In Vivo Evaluation of the Efficacy of Felinized 15H05 Anti IL-31 Antibodies in a Cat Pruritus Challenge Model

Based on the positive efficacy outcome using the mouse: feline 15H05 chimera described above, further work was done to increase the affinity and potency of felinized 15H05 (described above in section 1.8). Systematic substitution of the variable light chain feline frameworks in antibody feline 15H05 1.1 lead to the identification of Feline 15H05 1.1 FW2 having increased affinity to both feline and canine IL-31 compared to mouse 15H05 (FIG. 2). Combination of the heavy and light chains of Feline 15H05 1.1 FW2 into a single plasmid lead to the formation of ZTS-927 and ZTS-361 antibodies following production from HEK and CHO expression systems. The affinity and potency of both antibodies resulting from expression from a single plasmid are also described in FIGS. 2 and 3 respectively.

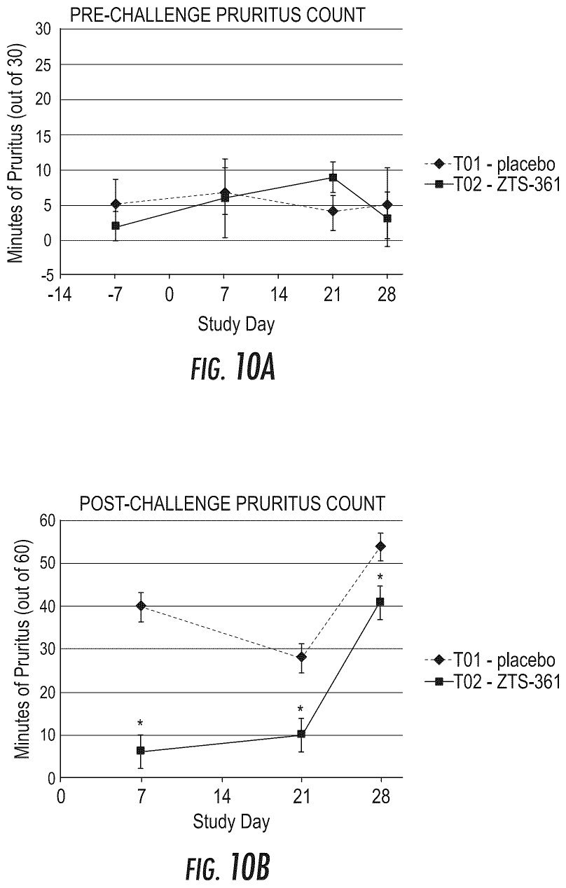

The efficacy of the fully felinized anti feline IL-31 mAb ZTS-361 was assessed for its ability to neutralize pruritic behavior in an IL-31 induced in vivo cat model. FIG. 10A shows the baseline pre-challenge pruritic behavior for the T01 vehicle placebo and T02 antibody ZTS-361 groups from day −7 through day 28 with day zero being the day of antibody administration to group T02. As shown in this graph, the variance of pruritic behavior scored for both T01 and T02 groups prior to IL-31 challenge varied little with the number of pruritic events observed between 0 and 10 within the 30 minute observation period. This study differed from the preliminary feline challenge model described above in section 1.13 in that on day zero cats were dosed with 4 mg/kg ZTS-361 subcutaneously without combination with feline IL-31 to generate a pre-bound complex. This represents a more rigorous assessment of efficacy as antibody ZTS-361 will be in circulation for seven days prior to the first IL-31 challenge requiring the antibody to have sufficient exposure to bind and neutralize circulating IL-31.

For this study, pruritic behavior was assessed on days 7, 21, and 28 for 1 hour following a 0.5 μg/kg intravenous challenge of the IL-31 protein. FIG. 10B shows the efficacy of antibody ZTS-361 demonstrating a significant reduction in pruritus observed on days 7 (p<0.0001), 21 (p<0.0027), and 28 (p<0.0238) following IL-31 challenge when compared to vehicle placebo control. Data from this challenge model support previous observations demonstrating the efficacy of mouse: feline 15H05 chimera and support the cell-based potency and relevance of the epitope on feline IL-31 recognized by the 15H05 CDRs. These data further support the ability of antibody ZTS-361 to neutralize pruritus induced by feline IL-31 in vivo and suggest this antibody may serve as a therapeutic in the treatment of IL-31 mediated disease in cats including atopic dermatitis.

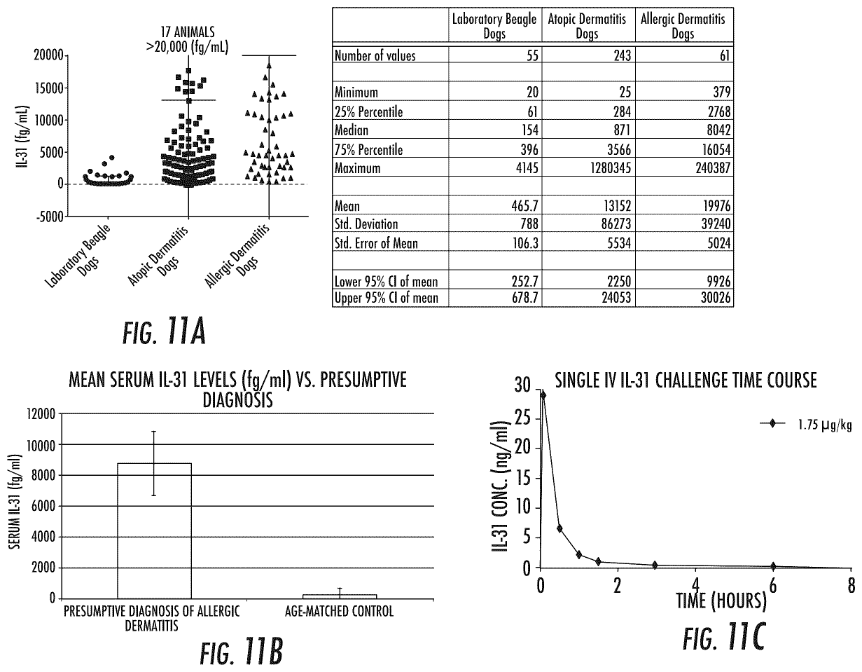

Recent data examining the plasma levels of IL-31 in client owned animals shows an increased amount of the cytokine in circulation among dogs with atopic and allergic dermatitis compared to normal laboratory beagles (FIG. 11A). A recent study was performed to determine serum IL-31 levels in cats with a presumptive diagnosis of allergic dermatitis (AD) from several different geographic regions in the USA. FIG. 11B shows the results from this assessment indicating that, like dogs with atopic and allergic dermatitis, 73 cats surveyed with this presumptive diagnosis had mean circulating IL-31 levels of 8799 fg/ml compared to 205 fg/ml in the 17 age-matched control cats. To understand the levels of canine IL-31 in a previous model development study, the pharmacokinetic profile of canine IL-31 was analyzed in dogs following administration of a subcutaneous dose of 1.75 μg/kg. FIG. 11C shows peak plasma levels within the first hour reaching a maximum of about 30 ng/ml and a maintained level of about 400 pg/ml at three hours. Based on these findings it is reasonable to believe that an intravenous dose of 0.5 μg/kg feline IL-31 used in this feline model will result in a circulating amount that is far excessive to that observed in the naturally occurring disease state for dogs and cats.

### 1.15. Analytical Methods Used for Advancement of Lead Felinized Anti IL-31 Antibodies

During the process of cell line development, various analytical methods are employed to ensure the antibody therapeutic can be manufactured in a consistent manor. Close attention to analytical methods which ensure (but are not limited to) product identity, purity, and potency are critical for consistent production of lead monoclonal antibodies and strong correlation to potency and safety outcomes in the target animal species. As antibodies are homodimers of two heterodimeric units held together through inter-chain disulphide bonds, any disruption in the pairing process can lead to non-uniformity of the protein drug product. Two analytical methods well suited to monitor the disassociation of antibodies are non-reducing (NR) sodium dodecyl sulphate (SDS) polyacrylamide gel electrophoresis (PAGE) and non-reducing (NR) capillary gel electrophoresis (CGE).

NR SDS-PAGE provides a convenient qualitative method for determining the mass of individual proteins species in a test sample. SDS hydrophobically associates with proteins uniformly conferring a net negative charge to the protein thus allowing separation of individual components based on mass. Following electrophoretic separation on a polyacrylamide gel, proteins are stained with a dye like Coomassie blue to allow for detection. Non-linearity of staining prevents absolute quantitation of individual protein bands but does allow for estimates using software capable of densiometric analysis (VersaDoc, Bio-Rad). Capillary gel electrophoresis, commonly known as CGE, subjects proteins to SDS again resulting in a uniform negative charge and dissociates non-covalent protein complexes. In the presence of an electric field, the SDS-coated proteins migrate toward the anode and are detected using ultraviolet light absorbance at a fixed wavelength of 220 nm. Separation is based on the size of the components in the sample within a capillary filled with replaceable SDS-polymer gel sieving matrix. In non-reducing CGE, an alkylating agent iodoacetamide (IAM) is added to minimize disulfide bond shuffling during sample preparation. The intact IgG is separated from any fragmented species, allowing quantitation of purity. Software for CGE analysis (examples are Empower or 32 Karat) utilizes the time corrected area (TCA) which is defined as the individual peak area by the migration time. Total TCA is defined as the sum of the TCAs for all peaks greater than or equal to 0.3%. Percent monomeric intact IgG and subspecies can thus be calculated based on their individual TCAs as a percent of the total TCA.

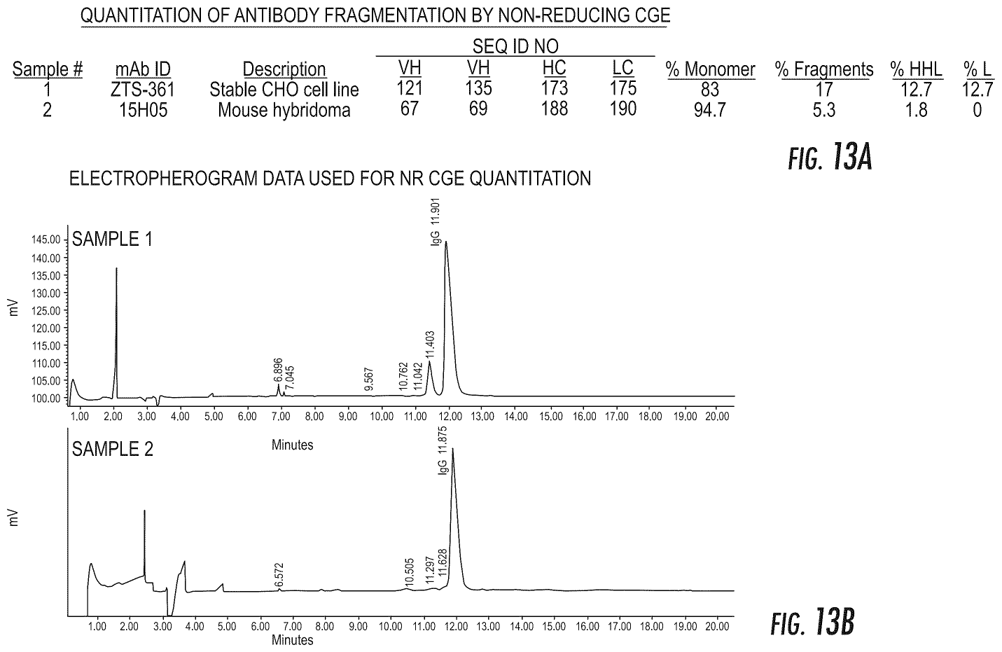

Given the promising in vivo efficacy data for ZTS-361 described for the feline model of pruritus (section 1.14), further characterization of various lots of antibody were determined using these described analytical methods. During this process, it was identified that stable pools expressing felinized anti IL-31 (ZTS-361) antibody had increased levels of lower molecular weight species (including free light chain) visible by Coomassie staining of an SDS-PAGE relative to the mouse progenitor hybridoma 15H05 (FIG. 12). Herein, intact, or intact monomer, refers to an IgG with two heavy chains and two light chains held together by interchain disulfide bonds with an expected molecular weight of ˜150 kDa. HHL refers to herein as “Heavy Heavy Light” an IgG with one light chain missing and an expected molecular weight of ˜125 kDa. HH refers to herein as “Heavy Heavy” an IgG with both light chains missing and an expected molecular weight of ˜100 kDa. HL refers to herein as “Heavy Light” an IgG with one heavy and one light chain and an expected molecular weight of ˜75 kDa. L refers to herein as “Light” an IgG with one light chain and an expected molecular weight of ˜25 kDa, also refered to herein as free light chain. Quantitative assessment of this same material using NR CGE revealed significantly less intact monomeric IgG (83%) for ZTS-361 when compared to the mouse progenitor 15H05 (94.7%) (FIG. 13A). FIG. 13B shows the electropherograms following resolution of samples by NR CGE. Data from these peaks at differing retention times were used to quantitate the percent TCA of total for sample 1 (ZTS-361) and sample 2 (mouse 15H05). The sum of the minor peaks with a lower molecular weight than the major intact IgG peak are used to calculate the percent fragments indicated in FIG. 13A (% fragments). Stable CHO pools producing ZTS-361 resulted in 17% of the final antibody product found as a fragmented form of the felinized IgG compared to only 5.3% with the mouse hybridoma 15H05.

To facilitate the understanding of this phenomenon, single clonal CHO cell isolates were derived from the ZTS-361 clonal pool to see if the percentage of intact IgG monomer varied between the individual clones. FIG. 14A shows a Coomassie-stained NR SDS-PAGE with purified antibody derived from cultures of 8 individual clones stably expressing ZTS-361. For comparison the lanes labeled 1 and 8 show a reference standard antibody known to have a high percentage of intact IgG monomer (˜97%). Qualitative densiometric determinations of band intensity are shown on the right of the Figure. Clonal variation of percent intact monomer ranges from 80.2% to 86% with an average of ˜82%. Likewise the quantitation of percent fragments ranged from 14.0% to 19.5% for the individual clones with an average of 17.5%. FIG. 14B shows the quantitative assessment of these individual clones using NR-CGE. Less variation is observed using this method with an average percent intact monomer of 86.3% and a percentage of lower molecular weight species of 13.7% observed across the 8 clones tested. While a high level of consistency was observed with the percentage of intact IgG monomer for ZTS-361 among the individual clones, it was of interest to understand why the overall level of intact IgG monomer was lower than that observed for the mouse version of the antibody. It is important to note that antibodies, not limited to but including felinized antibodies, produced from transient expression systems (examples are HEK and CHO cells) resulted in production of IgGs with a high level of the percent monomeric form (˜88% to ˜92%) (data not shown). Correspondingly the amount antibody produced from these transient cultures is significantly less than that from a stable CHO line. While not wishing to be bound by any one theory, the occurrence of fragmented antibody species seen with felinized and other species may be observed under conditions where the host cell is producing exceptionally high amounts of antibody and inherent limitations in the culture conditions and/or the antibodies molecular composition are observed.

1.16. Consideration of Primary Amino Acid Sequences of the IgG Kappa Light Chain Constant Domain from Multiple Species of Mammals

Analysis of potential limitations to the felinized antibody ZTS-361 began with consideration of the primary antibody sequence. ZTS-361 is composed of a heavy chain which includes a variable region (SEQ ID NO: 121; FEL_15H05_VH1), the corresponding nucleotide sequence for which is (SEQ ID NO: 122; FEL_15H05_VH1) combined with a feline IgG heavy chain constant region (SEQ ID NO: 173; Feline_HC_AlleleA_1) n the corresponding nucleotide sequence for which is (SEQ ID NO: 174; Feline_HC_AlleleA_1) and a light chain which includes a variable region (SEQ ID NO: 135; FEL-15H05-VL1_FW2), the corresponding nucleotide sequence for which is (SEQ ID NO: 136; FEL-15H05-VL1_FW2) combined with a feline IgG light chain constant region (SEQ ID NO: 175; Feline_LC_Kappa_G_minus), the corresponding nucleotide sequence for which is (SEQ ID NO: 176; Feline_LC_Kappa_G_minus). The functional properties of naturally-occurring feline antibody heavy chain constant regions has been previously described by Strietzel et al. (2014 Veterinary Immunology and Immunopathology April 15; 158(3-4): 214-223). We describe herein the cloning and expression of felinized antibody ZTS-361 using the heavy chain constant region (SEQ ID NO: 171; Feline_HC_AlleleA_1). SEQ ID NO: 171 Feline HC AlleleA 1 corresponds to feline IgG1a in Strietzel et al. 2014 supra and appears to be functionally equivalent to human IgG1. Comparison of these functional attributes and alignment of this heavy chain constant region with other constant regions from a diverse set of species did not reveal any obvious areas of concern that would lead to inefficiencies in formation of the intact IgG monomer (data not shown).

Similar analysis of the kappa constant chain used in ZTS-361 (SEQ ID NO: 175; Feline_LC_Kappa_G_minus) reveals a unique aspect of the kappa constant light chain diversity observed in at least the feline and canine sequences. Different species utilize the kappa light chain in their immunoglobulin repertoire at different frequencies. FIG. 15 shows representative c-terminal amino acids and corresponding nucleotides for several kappa light chain constants from the species indicated. The percentage of kappa light chain utilization by IgGs from the species depicted are according to; Canine, Feline, and Pig (Arun et al. 1996 Zentralbl Veterinarmed. November; 43(9):573-6), Mink (Bovkun et al. 1993 Eur J Immunol. August; 23(8):1929-34), Mouse (Woloschak et al. 1987 Mol Immunol. July; 24(7):751-7), Human (Barandun et al. 1976 Blood. January; 47(1):79-89). Routine generation of monoclonal antibodies is done using mice. As depicted in FIG. 15 the mouse utilizes the kappa light chain ˜95% of the time when compared to the lambda light chain. In contrast dogs and cats primarily use the lambda light chain in their immunoglobulin repertoire (9% and 8% respectively). By comparison, two non-human (pig and mink) and human mammals show a balanced utilization kappa and lambda light chain usage (50%, 46%, and ˜50% respectively). The position of the most c-terminal cysteine is annotated in this Figure and aligned across the different species. This cysteine is critical in the formation of the quaternary complex generated to form the intact IgG structure as it participates in the formation of the inter-chain covalent disulphide bond with the heavy constant chain. While not wishing to be bound to any one theory, it is of great interest to note that at least canine and feline kappa light chains contain multiple amino acids following the terminal cysteine. These additional amino acid residues have both polarity (glutamine) and charge (arginine, aspartic and glutamic acids). These residues are typically found in environments where they participate with hydrogen bond interactions which include, but are not limited to, interactions with the external aqueous environment. The nature and position of these additional residues beyond the terminal cysteine may interfere with the formation of an inter-chain disulphide bond that is necessary to form the IgG heterodimer. Two mammals (pig and mink) having fewer additional residues beyond the c-terminal cysteine and use kappa and lambda light chains at approximately equivalent ratios (50% and 46% respectively). Generation of recombinant forms of ZTS-361 with variations and deletions of amino acids at the c-terminus of the kappa light chain constant immediately following the cysteine at amino acid position 107 of SEQ ID NO: 175 were created using transient expression from HEK cells and tested for percent monomeric IgG by NR-CGE (data not shown). As mentioned previously, the production of unpaired light chain and lower molecular weight IgG complexes are more apparent under conditions whereby the antibody is being overproduced from a stable clonal cell. NR-CGE is however capable of detecting a differential in the amount of intact monomeric IgG versus lower molecular weight contaminants from cultures produced by transient transfection of cells (example HEK and CHO). This allows for a qualitative assessment of the percent monomeric IgG produced from transient cultures. This qualitative use of NR-CGE from transient cultures allowed for assay of multiple modifications and deletions to the c-terminus of the kappa constant chain of ZTS-361 for presence of percent monomeric IgG (data not shown). While not wishing to be bound by any one theory, deletion of the residues QRE from the c-terminus of the feline kappa constant chain appears to be the most optimal for production of monomeric recombinant feline IgG. Other additions to the c-terminus appear to be allowed. In general, one or two additional amino acids added beyond the cysteine at position 107 of Feline LC Kappa G minus (SEQ ID NO: 175) appear to be tolerated using qualitative assessment of percent monomer from transient produced IgG. Using these same qualitative assays it appears that even three additional amino acids added contiguously to the c-terminal end of the cysteine at position 107 in place of the native QRE amino acid residues may be tolerated if the amino acids have a minimal electrostatic charge influence. It is noted herein that the number and chemical properties of additional amino acids following the cysteine at position 107 of Feline LC Kappa G minus (SEQ ID NO: 175) will impact the efficient formation of the inter-chain disulphide bond between a kappa light chain constant and the corresponding Ig heavy chain constant. It is conceivable that modifications and/or deletions to this region may have advantageous effects of producing uniform intact IgG from a stable recombinant cell line.

The human and mouse kappa light chain c-terminal amino acid is the terminal cysteine therefore no additional amino acid residues are available to interact with the external environment during formation of the disulphide bond with the heavy chain constant. Therefore, it is hypothesized herein that these additional c-terminal residues in at least feline and canine kappa light chain constants present limitations to the formation of this disulphide bond that may not be ordinarily observed in nature due to the low abundance of these antibody species but may be highly relevant to the overproduction of these speciated forms in a laboratory setting. Such limitations may include the lack of formation of the disulphide bond between the c-terminus of the kappa light and the heavy chain constant chain leading to the presence of HHL, HH, HL, and L species previously described when recombinant antibody is produced from a stable cell line. These lower molecular weight species are undesirable in the production of a uniform drug product and their presence could be problematic from a quality and safety perspective.

Further to this observation are the codons which encode the amino acid residues in this region that appear below each amino acid letter in FIG. 15. In mammals there are three stop codons that signal termination polypeptide translation in the ribosome (TAA, TGA, and TAG). While not wishing to be bound by any one specific theory, it was observed that the majority of codons which encode for the amino acids following (and including) the c-terminal cysteine are one nucleotide away from a stop codon (FIG. 15, light grey letters in the codons). We hypothesize herein that somatic mutation at these various nucleotide positions may have led to the selection of an optimal immunoglobulin kappa constant chain length and amino acid composition that allows for efficient expression and correct chain pairing of an IgG kappa antibody. Subtle differences cannot be overlooked when assessing the allowance of appropriate amino acid which may exist at the c-terminus contiguous to the terminal cysteine. Noted herein is the observation that addition of residues EA on the pig light chain or Q on the mink light chain may have little to no deleterious impact on the formation of the inter-chain disulphide and expression of the intact IgG molecule. These species show equivalent use of kappa and lambda light chains and are described in the allowed region for acceptability of these additions. Pertaining to, but not limited to, canine and feline c-terminal kappa light chain residues, the distance from the c-terminal cysteine and the charged nature of the arginine and (aspartic and glutamic acid) residues is believed to have significant impact on the ability of a feline and canine antibody to efficiently and correctly form the covalent disulphide bond with its respective heavy chain.

### 1.17. Quaternary Structural Observations of the Feline Heavy Chain and Kappa Light Chain Interface

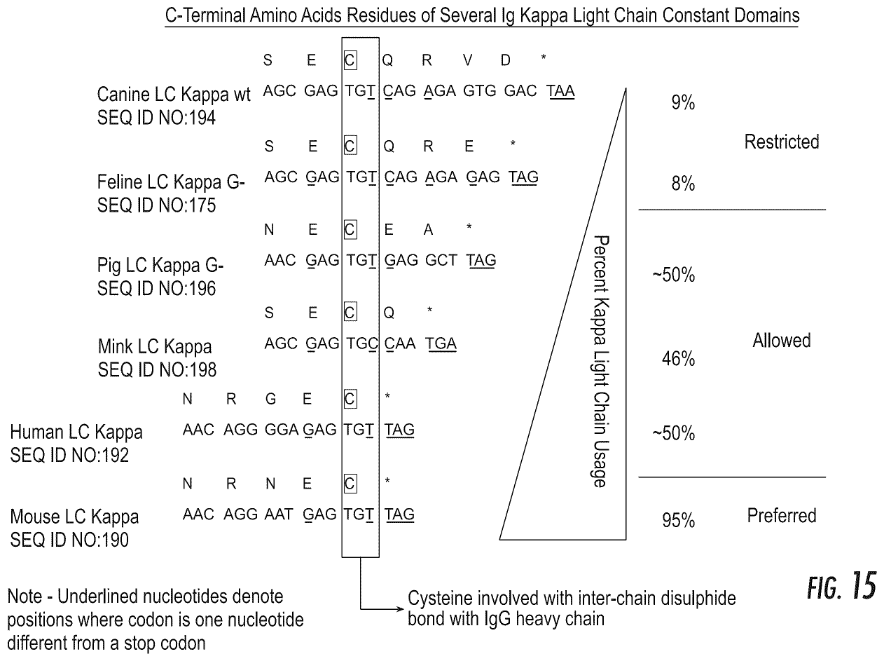

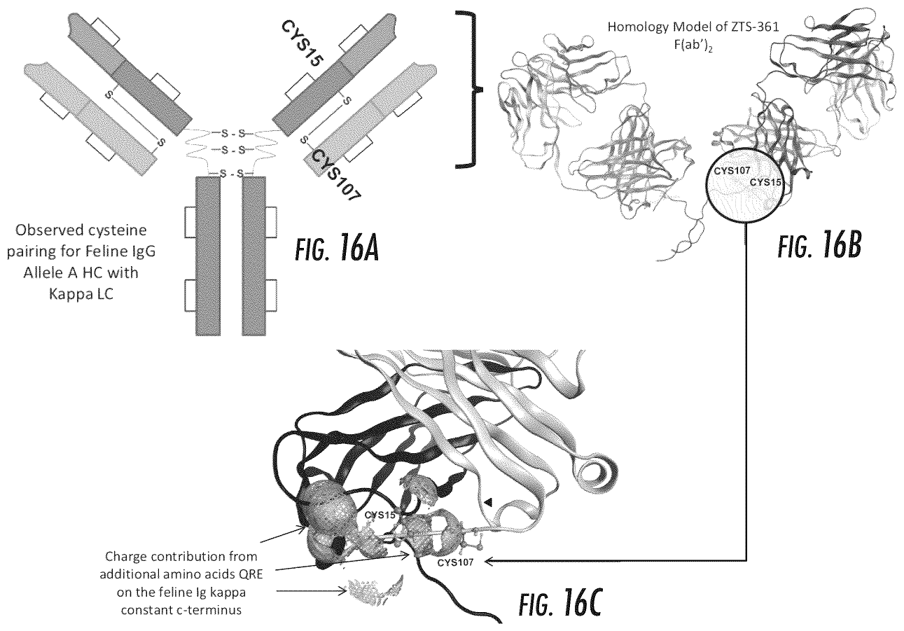

FIG. 16A is a pictoral representation of the expected structure of a feline IgG with a heavy and light chain equivalent to ZTS-361 (supportive analytical data not shown). The positions of intra and inter-chain disulphide bonds are shown highlighting the heavy chain cysteine (CYS15) and light chain cysteine (CYS107) on only one arm of the structure for simplification. CYS15, the depicted amino acid residue is position 15 of Feline HC AlleleA wt (SEQ ID NO: 171) and Feline HC AlleleA 1 (SEQ ID NO: 173), with the depicted nucleotide residue nos. being residue nos. 43-45 of Feline HC AlleleA wt (SEQ ID NO: 172) and 43-45 of Feline HC AlleleA 1 (SEQ ID NO: 174) respectively. CYS107, the depicted amino acid residue is position 107 of Feline LC Kappa G minus (SEQ ID NO: 175) and Feline LC Kappa G minus QRE minus (SEQ ID NO: 186), with the depicted nucleotide residue nos. being residue nos. 319-321 of Feline LC Kappa G minus (SEQ ID NO: 176) and 319-321 of Feline LC Kappa G minus QRE minus (SEQ ID NO: 187). As mentioned previously, the composition of feline ZTS-361 is functionally equivalent to human IgG1; however, the nature of the disulphide bonding pattern is more similar to that of a human IgG2 (data not shown). FIG. 16B shows a homology model representing the two F(ab′)₂ arms of ZTS-361 highlighting the approximate position of CYS15 and CYS107 for clarity using MOE software (Chemical Computing Group, Montreal, QC, Canada). FIG. 16C shows the contribution of additional electrostatic charges to the local environment that are contributed by the kappa light chain constant residues QRE that immediately follow CYS107. The charge from these three amino acids is represented as wire surface shells in this Figure. Homology models represent regions comprised of random coil structure with less accuracy than those found in ordered secondary structural elements like alpha helices and ordered beta sheets. This, however, is less of a consideration for CYS15 of the heavy chain as this region of the IgG constant domain is well defined by a wealth of structural data and the ordered nature of this region with the conserved IgG structure. Likewise, the CYS107 of the light chain has been resolved in numerous antibody structures and its proximity to CYS15 of the heavy chain can be appreciated. The addition of residues in the model beyond the terminal light chain cysteine can only be defined based on the geometry of adjacent residues and calculations of local energy minimums. This representation of the feline kappa light chain of ZTS-361 represents a best effort at producing a working model to generate a hypothesis for experimental design. Taken together, these results suggest that additional amino acid residues beyond the terminal cysteine in the kappa light chain of feline (and likely other species) is detrimental to efficient pairing with the heavy chain likely leading to mispairing and poor production of antibody.

1.18. Generation of Felinized Anti IL-31 Antibody ZTS-1505 with a Modified Kappa Constant C-Terminus

Given the potential limitations of consistently producing a homogeneous antibody preparation with ZTS-361 it was deemed necessary to find a solution to the lack of percent monomer produced. Towards, but not limited to, this effort was the generation of ZTS-1505, the heavy chain including a variable region (SEQ ID NO: 121; FEL_15H05_VH1), the corresponding nucleotide sequence for which is (SEQ ID NO: 122; FEL_15H05_VH1) combined with a feline IgG heavy chain constant region (SEQ ID NO: 173; Feline_HC_AlleleA_1), the corresponding nucleotide sequence for which is (SEQ ID NO: 174; Feline_HC_AlleleA_1). For ZTS-1505, the light chain includes a variable region (SEQ ID NO: 135; FEL-15H05-VL1_FW2), the corresponding nucleotide sequence for which is (SEQ ID NO: 136; FEL-15H05-VL1_FW2) combined with a feline IgG light chain constant region (SEQ ID NO: 186; Feline_LC_Kappa_G_minus_QRE_minus), the corresponding nucleotide sequence for which is (SEQ ID NO: 187; Feline_LC_Kappa_G_minus_QRE_minus). ZTS-1505 is identical to ZTS-361, except with the removal of the three additional residues QRE off the c-terminal most end of the light chain kappa constant in an effort to avoid the undesired effects of producing antibody with lower molecular weight species resulting from inefficient pairing with the kappa light chain. It is envisioned that removal of these residues is beneficial to the process of producing monomeric IgG. However, the present invention is not necessarily limited to this one modification in the kappa light chain since other changes to the kappa light chain have been made and appear to have a neutral or even beneficial effect based on preliminary experimental data in transient expression (data not shown).

FIG. 17A describes the variable and constant sequences used to construct antibodies ZTS-361 and ZTS-1505 highlighting the different light chain used for ZTS-1505. Creation of a stable CHO cell line expressing ZTS-1505 is described in section 1.9 of this application. Assessment of antibody quality was of high interest so individual clones from a stable CHO transfection were surveyed for their ability to produce monomeric antibody. FIG. 17B shows the results for quantitative assessment of these individual clones using NR-CGE. 23 individual stable CHO clones were assessed and a high level of consistency was observed in the percent monomer produced with an average of 90.3%. This represents a 4% increase from the average percent monomer produced with individual clonal isolates of ZTS-361 (FIG. 14B) and a 7.3% increase observed with the stable pool (FIG. 13A). FIG. 17C shows a direct comparison of an individual stable CHO cell line expressing antibody ZTS-361 and ZTS-1505 under different culture conditions. Of note, antibodies described in FIGS. 12, 13A and 13B, and 14A and 14B were grown in culture conditions equivalent to culture condition A in FIG. 17C and these conditions are described in section 1.9 of this application. Both cell lines were grown for 14 days and the percent viability, titer (antibody yield in g/L), and percent monomer by NR-CGE were determined. The percent viability was consistent across all the conditions tested with both cell lines. The percent monomer by NR-CGE showed a consistent 4-5% improvement in percent monomer comparing ZT-1505 to ZTS-361 indicating again that the kappa light chain constant in ZTS-1505 lacking the additional QRE residues had a positive effect on chain association between the feline heavy chain constant and kappa light chain. Even more striking was the improvement in the amount of ZTS-1505 antibody produced compared to ZTS-361. ZTS-1505 produced, on average, a 3.5 fold improvement in the production of antibody from a stable CHO cell when compared to ZTS-361 possessing the wildtype kappa light chain constant.

To insure the kappa light chain constant modification did not affect the affinity and potency of ZTS-1505, comparative analysis was performed using biacore and inhibition of IL-31 mediated pSTAT signaling in canine and feline cells. Table 1 below describes the results for biacore analysis using multiple species of IL-31 protein as a surface capture. These results show nearly identical affinity to canine, feline, and equine IL-31 protein was observed for ZTS-1505 possessing the light chain kappa constant modification when compared to ZTS-361 having the wildtype feline kappa constant c-terminus. Also described here is the expected binding phenotype for ZTS-1505 to the feline 15H05 and feline 11E12 mutants described section 1.2 of this application. Modification of the c-terminus of the kappa constant chain did not alter the selectivity for the epitope as demonstrated by the lack of ZTS-1505 binding to the feline 15H05 mutant protein and the retention of binding to the feline 11E12 mutant, equivalent to ZTS-361.

TABLE 1

| | | | | | | Biacore KD (M) IL-31 Species | | | | |
|---|---|---|---|---|---|---|---|---|---|---|
| | | | | | | Capture Protein | | | | |
| | | | | | | SEQ ID NO | | | | |
| | | | | | | | | | 163 | 161 |
| | | | | | | | | | Feline | Feline |
| | | Antibody SEQ ID NO | | | | 157 | 155 | 165 | 15H05 | 11E12 |
| mAb ID | Description | VH | VL | HC | LC | Feline | Canine | Equine | mutant | mutant |
| 15H05 | Mouse hybridoma | 67 | 69 | 188 | 190 | 3.06E−10 | 1.91E−12 | 3.06E−10 | 2.39E−08 | 1.15E−08 |
| ZTS-361 | Stable CHO cell line | 121 | 135 | 173 | 175 | 6.25E−10 | 2.41E−12 | 6.25E−10 | no binding | 1.72E−08 |
| ZTS-1505 | Stable CHO cell line | 121 | 135 | 173 | 186 | 5.75E−10 | 1.83E−12 | 5.75E−10 | no binding | 1.95E−08 |

Table 2 below shows the cellular potency data comparing ZTS-1505 with ZTS-361. The light chain kappa constant modification for ZTS-1505 did not impact the ability of this antibody to inhibit cellular pSTAT signaling induced by canine and feline IL-31 on canine DH82 and feline FCWF-4 cells respectively as indicated by the comparable IC₅₀ values.

TABLE 2

| | Inhibition of IL-31 induced pSTAT3 IC₅₀ (μg/ml) | |
|---|---|---|
| | Canine IL-31 on | Feline IL-31 on |
| mAb ID | Canine DH82 cells | Feline FCWF-4 cells |
| Mouse 15H05 | 11.17 | 6 |
| ZTS-361 | 22.99 | 8.85 |
| ZTS-1505 | 21.81 | 4.53 |

Taken together these results suggest the removal, or modification, of the c-terminal end of the kappa light chain from those species whose native germline encodes these additional residues is beneficial to both the production of homogeneous recombinant antibody for these species and beneficial to the amount of antibody produced from a stable cell line (e.g., yield improvement). Furthermore such modifications, while enhancing the quality and quantity of the antibody produced, have no negative impact on affinity and potency of the antibody to the IL-31 target protein. It is noted herein that these factors may be beneficial to the production of recombinant antibodies for therapeutic use in multiple species.

1.19. Confirmation of NR CGE Results with Anti IL-31 mAb 1505 and Further Demonstration for the Utility of the Modified Kappa Constant C-Terminus with an Anti NGF Antibody

It was of interest to determine if the increased production of percent monomeric IgG was applicable to different antibodies whose protein targets are dissimilar to IL-31. To accomplish this, an additional set of stable CHO cell line pools were generated with felinized antibodies recognizing the feline beta-nerve growth factor (NGF) (ZTS-768 and ZTS-943). The sequence for antibody ZTS-768 heavy chain is (SEQ ID NO: 220; ZTS_768_VH), the corresponding nucleotide sequence for which is (SEQ ID NO: 221; ZTS_768_VH) combined with a feline IgG heavy chain constant region (SEQ ID NO: 171; Feline_HC_AlleleA_wt), the corresponding nucleotide sequence for which is (SEQ ID NO: 172; Feline_HC_AlleleA_wt). The sequence for antibody ZTS-943 heavy chain is (SEQ ID NO: 224; ZTS_943_VH), the corresponding nucleotide sequence for which is (SEQ ID NO: 225; ZTS_943_VH) combined with a feline IgG heavy chain constant region (SEQ ID NO: 171; Feline_HC_AlleleA_wt), the corresponding nucleotide sequence for which is (SEQ ID NO: 172; Feline_HC_AlleleA_wt). For ZTS-768 the light chain variable is (SEQ ID NO: 222; ZTS_768_VL), the corresponding nucleotide sequence for which is (SEQ ID NO: 223; ZTS_768_VL). For ZTS-768, this light chain variable region is combined with a feline IgG light chain constant region (SEQ ID NO: 175; Feline_LC_Kappa_G_minus), the corresponding nucleotide sequence for which is (SEQ ID NO: 176; Feline_LC_Kappa_G_minus). For ZTS-943, the light chain variable region is (SEQ ID NO: 226; ZTS_943_VL), the corresponding nucleotide sequence for which is (SEQ ID NO: 227; ZTS_943_VL) combined with a feline IgG light chain constant region (SEQ ID NO: 186; Feline_LC_Kappa_G_minus_QRE_minus), the corresponding nucleotide sequence for which is (SEQ ID NO: 187; Feline_LC_Kappa_G_minus_QRE_minus). For a direct comparison to anti IL-31 antibodies, stable pools of ZTS-361 and ZTS-1505 were also generated using identical culture and purification conditions to the anti NGF antibodies.

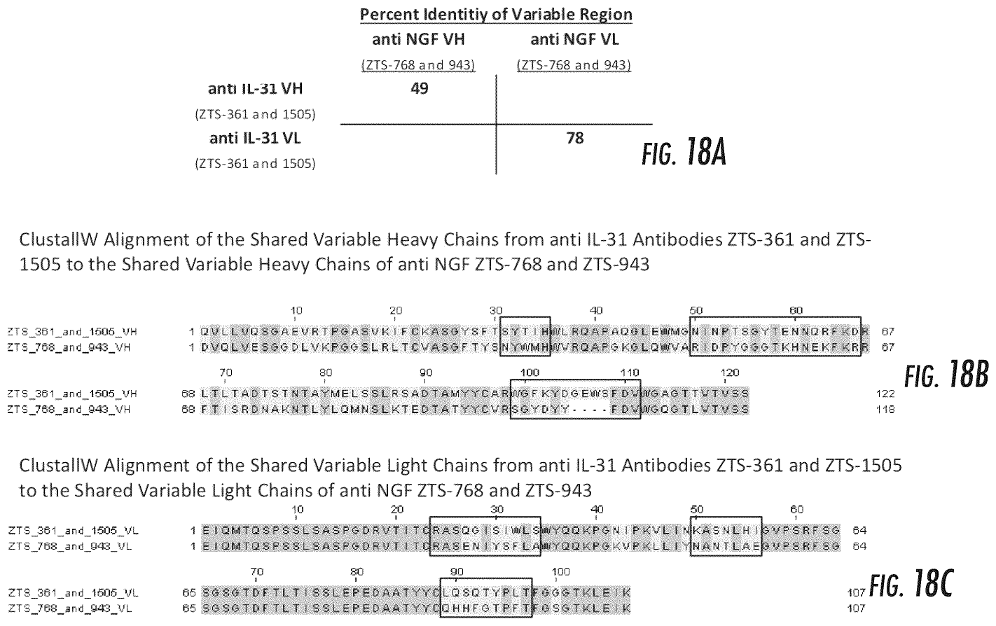

NR CGE was used to determine the percent monomeric intact IgG and subspecies following production and purification of the four antibodies described above. By comparison of these antibodies generated and purified using identical methods it was possible to assess the utility of incorporating the modified kappa constant C-terminus between structurally disparate antibodies recognizing distinct protein targets. FIG. 18A shows the percent identity comparing the variable regions of these anti feline NGF antibodies to anti IL-31 calculated using the ClustallW software. FIGS. 18B and 18C show the alignment of the anti feline IL-31 and NGF antibodies variable heavy and light chains respectively with the CDRs outlined with boxes. Clearly the anti IL-31 and anti NGF antibodies are distinct from one another with a lack of overall identity, especially within the antigen binding regions highlighted as CDRs.

FIG. 19 shows the results from the NR CGE comparing anti feline IL-31 and anti feline NGF antibodies with and without the modified kappa constant C-terminus. Consistent with previous findings, the anti IL-31 antibody containing the wildtype kappa light chain (ZTS-361) was observed to have 80.74% monomeric IgG with a predominant 8.71% of the HHL subspecies. A clear improvement in the percent monomer was again observed following the removal of the three C-terminal residues to generate ZTS-1505 (89.17% monomer with 5.9% HHL). NR CGE analysis of purified IgG from the anti feline NGF mAb containing the wildtype kappa light chain (ZTS-768) resulted in a similar amount of monomer and subspecies being isolated (80.81% monomer with 12.33% HHL) as compared to anti IL-31 ZTS-361. Notably, the same pattern was observed following removal of the C-terminal residues from the anti feline NGF antibody yielding 88.59% monomer with the predominant subspecies being 5.98% HHL.

These results suggest a structural distinction exists between a feline IgG protein which contains the wildtype amino acid residues (QRE) on the C-terminus of the kappa light chain versus an IgG with these residues removed. The experimental results described herein support that the use of this kappa light chain modification results in the production of monomeric feline IgGs with a reduced amount of subspecies contaminants. Further to this, the results herein clearly demonstrate that this method applies to structurally disparate antibodies which recognize completely distinct targets and therefore this modification will likely be applicable to the broad genus of feline antibodies as well as other mammalian antibodies possessing additional C-terminal amino acids on the kappa light chain constant region. While not wishing to be bound by any one theory, this light chain modification appears to result in a higher fidelity of immunoglobulin chain pairing during the induced production from stable CHO cell lines resulting in a higher amount of monomeric IgG and potentially a higher overall antibody yield. Both of these attributes are highly desirable from the standpoint of manufacturing commercial grade antibody therapeutics.

1.20 Identification of Anti Equine IL-31 Antibodies which Bind to an Equivalent Region on the Equine IL-31 Protein Compared to mAb 15H05 Binding to Feline IL-31

Given the promising in vivo efficacy in the feline model of pruritus using antibodies from the mouse 15H05 lineage described herein, it was desirable to identify novel antibody substrates which bind to a similar region on the equine ortholog of the IL-31 protein. Towards this end, mice were immunized with recombinant equine IL-31 (SEQ ID No. 165) for the purpose of identifying antibodies that bind to equine IL-31.

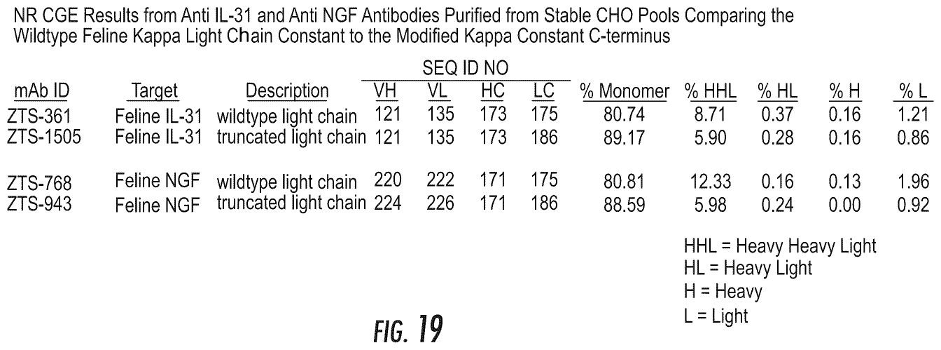

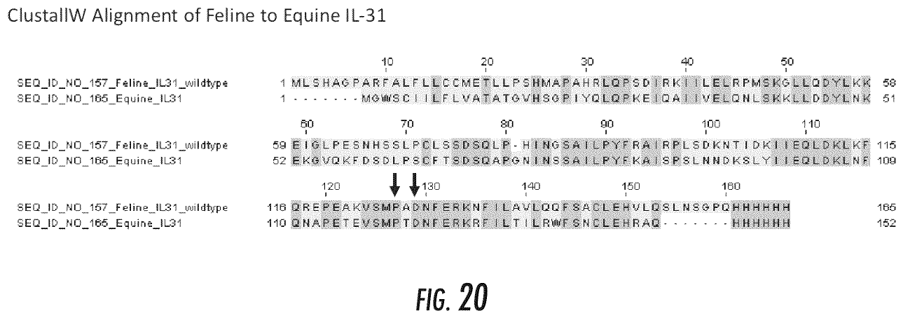

Serum antibody titers from immunized animals were determined using an ELISA as described previously. Donor splenocytes from a single responsive mouse were used for fusion and hybridoma supernatants were screened for antibodies that bind to the equine IL-31 protein by ELISA. This resulted in the identification of two mouse antibodies that bind to a region on the equine IL-31 protein comparable to the binding site of antibody 15H05 on feline IL-31. Section 1.10 of this application describes the characterization of the binding site of antibody 15H05 with illustration of the approximate binding site on a homology model of feline IL-31 in FIG. 6B. FIG. 20 shows an alignment of feline IL-31 wildtype (SEQ ID No. 157) with equine IL-31 (SEQ ID No. 165) using the ClustallW software. The arrows above the alignment indicate residues P126 and D128 which are described in section 1.10 as being contained within the binding region of antibody 15H05 to feline IL-31 at site 2 (FIG. 6B). These two anti equine IL-31 antibodies that share this binding site in the equine IL-31 protein are 04H07 and 06A09.

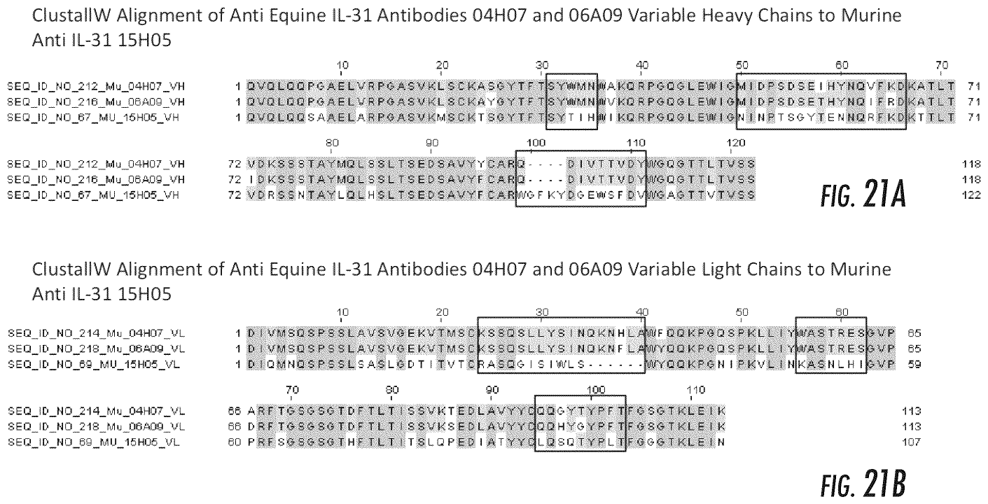

Anti equine IL-31 04H07 and 06A09 were further subcloned to generate a hybridoma producing homogeneous antibody and for sequencing of the variable heavy and light chains. The mouse anti IL-31 variable sequences determined for antibody 04H07 are as follows, 04H07 variable heavy chain (SEQ ID NO: 212; Mu_04H07_VH), the corresponding nucleotide sequence for which is (SEQ ID NO: 213; Mu_04H07_VH), 04H07 variable light chain (SEQ ID NO: 214; Mu_04H07_VL), the corresponding nucleotide sequence for which is (SEQ ID NO: 215; Mu_04H07_VL). 06A09 variable heavy chain (SEQ ID NO: 216; Mu_06A09_VH), the corresponding nucleotide sequence for which is (SEQ ID NO: 217; Mu_06A09_VH), 06A09 variable light chain (SEQ ID NO: 218; Mu_06A09_VL), the corresponding nucleotide sequence for which is (SEQ ID NO: 219; Mu_06A09_VL). FIGS. 21A and 21B show an alignment of the variable heavy (FIG. 21A) and light (FIG. 21B) chains of 04H07 and 06A09 compared to mouse antibody 15H05 using ClustallW. For comparison, the location of each of the six CDRs are outlined with boxes. Anti equine IL-31 antibodies 04H07 and 06A09 are highly similar to one another likely emerging from a common clonal lineage. The anti feline IL-31 antibody 15H05 is clearly distinct in the amino acid sequences of the CDRs and the lengths of CDRH3 and CDRL1 compared to anti equine IL-31 04H07 and 06A09. It is of interest that the binding site of antibody 15H05 to feline IL-31 is conserved when compared to the equine protein (FIG. 20 arrows). These results further exemplify that structurally distinct CDRs are capable of recognizing a common epitope region on two IL-31 orthologs.

### 1.21. Alanine Scanning of the CDRs on Anti IL-31 Antibody ZTS-1505

The region of an antibody responsible for antigen recognition represents the paratope. A paratope is created by a combination of amino acids in the complementarity determining regions (CDRs) of both the heavy and light chain variable regions. The binding between antibody and antigen is often mediated by side chains of CDR residues with side chains or carbohydrate moieties of the antigen. To help define critical side chains involved in antibody recognition alanine scanning mutagenesis was performed on each CDR residue in both the heavy and light chain. These mutants were then individually tested for the ability to bind feline IL-31 using the biacore and evaluated for inhibition of IL-31-mediated signaling in the FCWF-4 cell based assay.

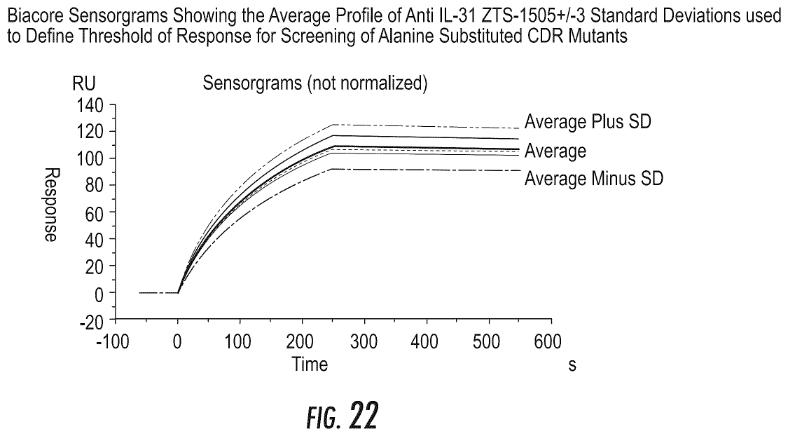

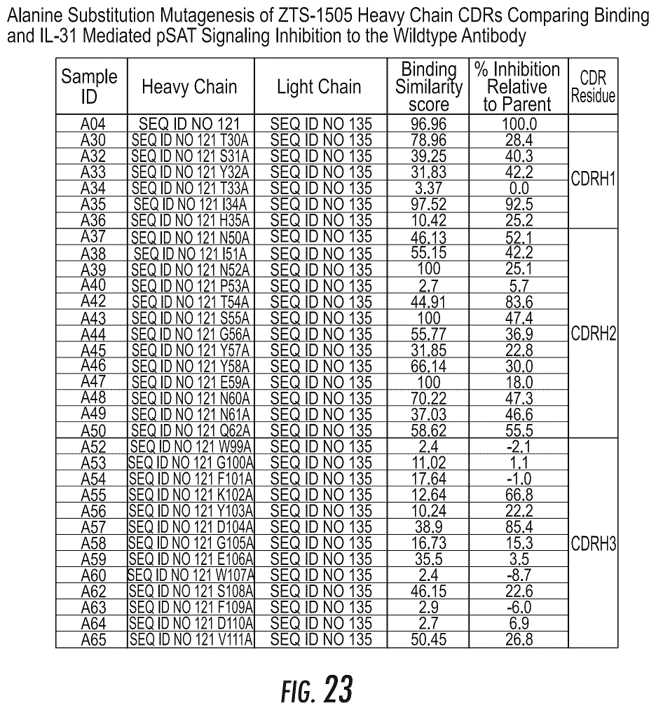

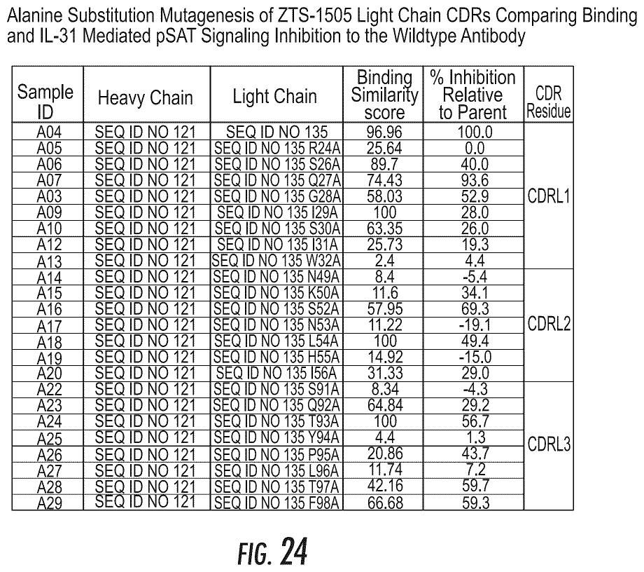

To determine the relative affinity of the alanine scanning mutant mAbs to the parent mAb binding profiles to feline IL-31 coated chips was determined at 100 nM using a Biacore T200. The mean response unit of four replicates of the parent mAb+/−3 standard deviations was used to generate parameters to define a threshold of response units comprising both the on- and off-rates antibody binding (FIG. 22). The percentage of data points for each mutant fell within this threshold was then used to define a “% similarity score” (FIG. 23). The similarity score resulting from the substitution of alanine at each heavy and light chain CDR position in antibody ZTS-1505 is shown in FIGS. 23 and 24 respectively.

To determine the relative activity of the ZTS-1505 mutant antibodies in the cell based assay each antibody was assessed for its ability to inhibit feline-IL-31-mediated STAT phosphorylation in Fcwf-4 cells at 15 ug/mL. Relative inhibition of STAT phosphyrlation was determine by evaluating the % inhibition of stat phosphorylation of each mutant relative to parent. Results from substitiution of alanine at each CDR position of ZTS-1505 for the heavy and light chain are shown as “percent inhibition relative to parent” in FIGS. 23 and 24 respectively. By evaluating the effect that individual alanine substitutions have on binding affinity and cell based activity the side chains of each CDR amino acid residue can be individually assessed for its role in antigen recognition. CDR residues that can be changed to alanine and retain activity are likely to be amenable to a variety of amino acid substitutions and represent residues that are not critical in antigen recognition. Based on these data, at least residue 4 (I) from SEQ ID NO 1; residues 1-3 (NIN), 5-7 (TSG), 9-11 (TEN) and 13 (Q) from SEQ ID NO 2; residues 4 (K), 6 (D) and 13 (V) from SEQ ID NO 3 are non-critical residues for antigen binding in heavy chain CDR 1, 2 and 3, respectively. Light chain residues CDR residues that are not critical for binding include residues 3-7 (SQGIS) from SEQ ID NO 4, residue 3 (S) and 5 (L) from SEQ ID NO 5, and residue 4 (Q), 5 (T) and 9 (T) in SEQ ID NO 6 from CDRL1, 2, and 3, respectively.

### 1.22. Binding Affinity and Cellular Potency of Two Felinized Versions of the ZIL8 Antibody

Section 1.6 above of this example section describes the identification of a canine antibody that recognizes feline IL-31 termed ZIL8. Initial screening revealed that this antibody is capable of binding the feline IL-31 protein, however its binding is affected by the 15H05 mutation. It was therefore of interest to pursue a felinized form of this antibody for use as a therapeutic in cats. Towards this end, a felinization strategy was followed as previously described in section 1.8 of this application. Grafting of the ZIL8 CDRS onto the appropriate feline frameworks yielded antibodies ZTS-5864 and ZTS-5865. The sequence for antibody ZTS-5864 heavy chain is (SEQ ID NO: 228; ZTS_5864_VH), the corresponding nucleotide sequence for which is (SEQ ID NO: 229; ZTS_5864_VH) combined with a feline IgG heavy chain constant region (SEQ ID NO: 173; Feline_HC_AlleleA_1), the corresponding nucleotide sequence for which is (SEQ ID NO: 174; Feline_HC_AlleleA_1). For ZTS-5864 the light chain variable is (SEQ ID NO: 230; ZTS_5864_VL), the corresponding nucleotide sequence for which is (SEQ ID NO: 231; ZTS_5864_VL). For ZTS-5864, this light chain variable region is combined with a feline IgG light chain constant region (SEQ ID NO: 236; Feline_LC_Lambda), the corresponding nucleotide sequence for which is (SEQ ID NO: 237; Feline_LC_Lambda). The sequence for antibody ZTS-5865 heavy chain is (SEQ ID NO: 232; ZTS_5865_VH), the corresponding nucleotide sequence for which is (SEQ ID NO: 233; ZTS_5865_VH) combined with a feline IgG heavy chain constant region (SEQ ID NO: 173; Feline_HC_AlleleA_1), the corresponding nucleotide sequence for which is (SEQ ID NO: 174; Feline_HC_AlleleA_1). For ZTS-5865 the light chain variable is (SEQ ID NO: 234; ZTS_5865_VL), the corresponding nucleotide sequence for which is (SEQ ID NO: 235; ZTS_5865_VL). For ZTS-5865, this light chain variable region is combined with a feline IgG light chain constant region (SEQ ID NO: 236; Feline_LC_Lambda), the corresponding nucleotide sequence for which is (SEQ ID NO: 237; Feline_LC_Lambda).

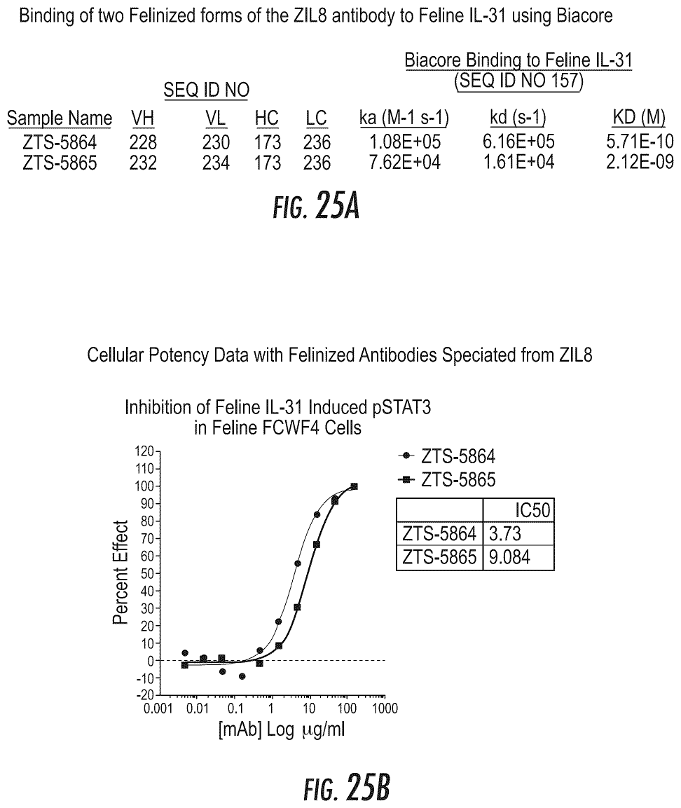

FIGS. 25A and 25B show this affinity and cellular potency of the two felinized antibodies ZTS-5864 and ZTS-5865. Both antibodies have high affinity to feline IL-31 with ZTS-5864 having an approximately 4 fold increase in affinity (KD (M)) when compared to ZTS-5864. Cellular potency was assessed using feline IL-31 to stimulate pSTAT3 signalling in FCFW4 cells. IC₅₀ vlaues were calculated for each antibody as described previously in this application. ZTS-5864 is approximately 3 fold more potent when comparing its IC₅₀ value to that of ZTS-5865. It is worth noting that both antibodies are considered potent with IC₅₀s in the range of those antibodies previously described herein from the 15H05 lineage (FIG. 3). The relevance of these potencies using pSTAT3 singalling from feline FCFW4 cells were previously qualified with positive in vivo efficacy results using chimeric and felinized antibodies in a feline model of pruritus (FIG. 9 and FIGS. 10A and 10B).

### 1.23 In Vivo Evaluation of the Efficacy of Felinized ZTS-5864 Anti IL-31 Antibody in a Cat Pruritus Challenge Model

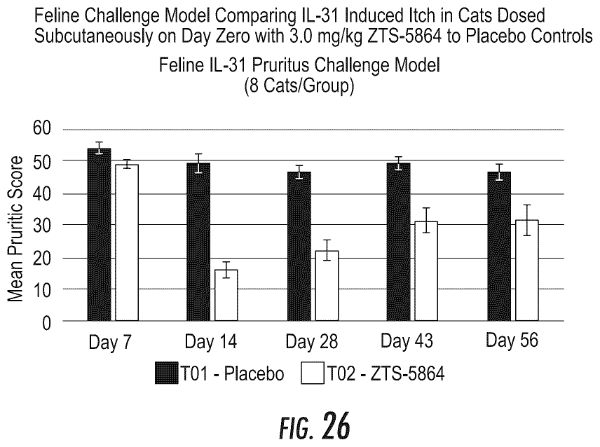

In vivo efficacy of ZTS-5864 was assessed in a feline IL-31 model of induced pruritus as described previously in section 1.14 of this example section. FIG. 26 shows the results from this study. Predose (day −7) indicates that the animals in T01 and T02 groups have an equivalent pruritic response to IL-31 challenge prior to dosing. Very little change was observed in the T01 pruritic response over the course of the study out to 56 days. In contrast ZTS-5864 (3.0 mg/mg) given subcutaneously on day zero attenuated the pruritic effect of the IL-31 challenge throughtout the entire course of study to day 56 (FIG. 26, T02). The continued decrese in mean pruritic score for T02 at day 56 indicates the antibody will likely maintain efficacy beyond this timepoint. These results accentuate the robust criteria used for the selection of anti feline IL-31 antobdies in this application. These results further support the positioning of these speciated antibodies as therapeutics for the treatment of IL-31 mediated disorders in cats.

What is claimed is:

1. A method of improving the consistency and/or quality of a feline antibody, the method comprising:
expressing nucleotide sequence encoding a feline IgG kappa light chain and nucleotide sequence encoding a feline IgG heavy chain in a host cell to produce a feline antibody, wherein the nucleotide sequence encoding the feline IgG kappa light chain comprises a kappa light chain constant nucleotide sequence in which sequence encoding a C-terminal QRE sequence otherwise present in a wild-type feline IgG kappa light chain constant region is absent.

2. The method of claim 1, wherein the method comprises:
a) providing nucleotide sequence encoding a wild-type feline IgG kappa light chain constant region of a feline antibody, wherein said wild-type feline kappa light chain constant region comprises a C-terminal amino acid sequence of QRE;
b) removing sequence encoding the C-terminal QRE in the nucleotide sequence in a) to form a revised kappa light chain constant nucleotide sequence;
c) combining the revised kappa light chain constant nucleotide sequence from b) with nucleotide sequence encoding a feline IgG kappa light chain variable region to form nucleotide sequence encoding a complete feline IgG kappa light chain; and
d) expressing the nucleotide sequence encoding the complete feline IgG kappa light chain from c) and nucleotide sequence encoding a feline IgG heavy chain in a host cell to produce a feline antibody in which the C-terminal QRE sequence which would otherwise be present in the wild-type feline IgG kappa light chain constant region is absent.

3. The method of claim 1, wherein improving the consistency and/or quality of the feline antibody comprises reducing the levels of free IgG kappa light chain, thereby increasing the percentage of intact feline IgG antibody monomer.

4. The method of claim 1, wherein the nucleotide sequence encoding the feline IgG kappa light chain and the nucleotide sequence encoding the feline IgG heavy chain are carried on the same vector used to transform the host cell.

5. The method of claim 1, wherein the nucleotide sequence encoding the feline IgG kappa light chain and the nucleotide sequence encoding the feline IgG heavy chain are carried on separate vectors used to transform the host cell.

6. The method of claim 1, wherein the feline antibody specifically binds to a target involved in a cytokine and/or growth factor-mediated disorder.

7. The method of claim 6, wherein the feline antibody specifically binds to feline IL-31.

8. The method of claim 6, wherein the feline antibody specifically binds to feline NGF.

9. The method of claim 1, wherein the feline antibody comprises a kappa light chain constant region having the sequence
(SEQ ID NO: 186)
RSDAQPSVFLFQPSLDELHTGSASIVCILNDFYPKEVNVKWKVDGVVQN
KGIQESTTEQNSKDSTYSLSSTLTMSSTEYQSHEKFSCEVTHKSLASTL
VKSFQRSEC.

10. The method of claim 1, wherein the nucleotide sequence encoding the feline IgG kappa light chain comprises a kappa light chain constant nucleotide sequence in which sequence encoding the absent C-terminal QRE sequence is substituted with up to three contiguous amino acids which have a minimal electrostatic charge influence.

11. The method of claim 10, wherein the up to three contiguous amino acids which have a minimal electrostatic charge influence are added to the C-terminal Cysteine at position 107 of SEQ ID NO: 186.


SEQUENCE LISTING (237 sequences)

SEQ ID NO: 1 - protein, 5 aa, Mus musculus
SYTIH

SEQ ID NO: 2 - protein, 17 aa, Mus musculus
NINPTSGYTENNQRFKD

SEQ ID NO: 3 - protein, 13 aa, Mus musculus
WGFKYDGEWSFDV

SEQ ID NO: 4 - protein, 11 aa, Mus musculus
RASQGISIWLS

SEQ ID NO: 5 - protein, 7 aa, Mus musculus
KASNLHI

SEQ ID NO: 6 - protein, 9 aa, Mus musculus
LQSQTYPLT

SEQ ID NO: 7 - protein, 5 aa, Mus musculus
YYDIN

SEQ ID NO: 8 - protein, 17 aa, Mus musculus
WIFPGDGGTKYNETFKG

SEQ ID NO: 9 - protein, 14 aa, Mus musculus
ARGGTSVIRDAMDY

SEQ ID NO: 10 - protein, 15 aa, Mus musculus
RASESVDNYGISFMH

SEQ ID NO: 11 - protein, 7 aa, Mus musculus
RASNLES

SEQ ID NO: 12 - protein, 9 aa, Mus musculus
QQSNKDPLT

SEQ ID NO: 13 - protein, 5 aa, Canis sp.
  source 1..5: note = Canis familiaris
SYGMS

SEQ ID NO: 14 - protein, 17 aa, Canis sp.
  source 1..17: note = Canis familiaris
HINSGGSSTYYADAVKG

SEQ ID NO: 15 - protein, 15 aa, Canis sp.
  source 1..15: note = Canis familiaris
VYTTLAAFWTDNFDY

SEQ ID NO: 16 - protein, 13 aa, Canis sp.
  source 1..13: note = Canis familiaris
SGSTNNIGILAAT

SEQ ID NO: 17 - protein, 7 aa, Canis sp.
  source 1..7: note = Canis familiaris
SDGNRPS

SEQ ID NO: 18 - protein, 11 aa, Canis sp.
  source 1..11: note = Canis familiaris
QSFDTTLDAYV

SEQ ID NO: 19 - protein, 5 aa, Canis sp.
DYAMS

SEQ ID NO: 20 - protein, 17 aa, Canis sp.
GIDSVGSGTSYADAVKG

SEQ ID NO: 21 - protein, 8 aa, Canis sp.
GFPGSFEH

SEQ ID NO: 22 - protein, 13 aa, Canis sp.
TGSSSNIGSGYVG

SEQ ID NO: 23 - protein, 7 aa, Canis sp.
YNSDRPS

SEQ ID NO: 24 - protein, 10 aa, Canis sp.
SVYDRTFNAV

SEQ ID NO: 25 - protein, 5 aa, Canis sp.
SYDMT

SEQ ID NO: 26 - protein, 17 aa, Canis sp.
DVNSGGTGTAYAVAVKG

SEQ ID NO: 27 - protein, 9 aa, Canis sp.
LGVRDGLSV

SEQ ID NO: 28 - protein, 11 aa, Canis sp.
SGESLNEYYTQ

SEQ ID NO: 29 - protein, 7 aa, Canis sp.
RDTERPS

SEQ ID NO: 30 - protein, 10 aa, Canis sp.
ESAVDTGTLV

SEQ ID NO: 31 - protein, 5 aa, Canis sp.
TYVMN

SEQ ID NO: 32 - protein, 17 aa, Canis sp.
SINGGGSSPTYADAVRG

SEQ ID NO: 33 - protein, 8 aa, Canis sp.
SMVGPFDY

SEQ ID NO: 34 - protein, 11 aa, Canis sp.
SGESLSNYYAQ

SEQ ID NO: 35 - protein, 7 aa, Canis sp.
KDTERPS

SEQ ID NO: 36 - protein, 10 aa, Canis sp.
ESAVSSDTIV

SEQ ID NO: 37 - protein, 5 aa, Canis sp.
SYAMK

SEQ ID NO: 38 - protein, 17 aa, Canis sp.
TINNDGTRTGYADAVRG

SEQ ID NO: 39 - protein, 15 aa, Canis sp.
GNAESGCTGDHCPPY

SEQ ID NO: 40 - protein, 11 aa, Canis sp.
SGESLNKYYAQ

SEQ ID NO: 41 - protein, 7 aa, Canis sp.
KDTERPS

SEQ ID NO: 42 - protein, 10 aa, Canis sp.
ESAVSSETNV

SEQ ID NO: 43 - protein, 5 aa, Canis sp.
TYFMS

SEQ ID NO: 44 - protein, 17 aa, Canis sp.
LISSDGSGTYYADAVKG

SEQ ID NO: 45 - protein, 7 aa, Canis sp.
FWRAFND

SEQ ID NO: 46 - protein, 14 aa, Canis sp.
GLNSGSVSTSNYPG

SEQ ID NO: 47 - protein, 7 aa, Canis sp.
DTGSRPS

SEQ ID NO: 48 - protein, 10 aa, Canis sp.
SLYTDSDILV

SEQ ID NO: 49 - protein, 5 aa, Canis sp.
DRGMS

SEQ ID NO: 50 - protein, 17 aa, Canis sp.
YIRYDGSRTDYADAVEG

SEQ ID NO: 51 - protein, 8 aa, Canis sp.
WDGSSFDY

SEQ ID NO: 52 - protein, 16 aa, Canis sp.
KASQSLLHSDGNTYLD

SEQ ID NO: 53 - protein, 7 aa, Canis sp.
KVSNRDP

SEQ ID NO: 54 - protein, 9 aa, Canis sp.
MQAIHFPLT

SEQ ID NO: 55 - protein, 5 aa, Canis sp.
SYVMT

SEQ ID NO: 56 - protein, 17 aa, Canis sp.
GINSEGSRTAYADAVKG

SEQ ID NO: 57 - protein, 10 aa, Canis sp.
GDIVATGTSY

SEQ ID NO: 58 - protein, 11 aa, Canis sp.
SGETLNRFYTQ

SEQ ID NO: 59 - protein, 7 aa, Canis sp.
KDTERPS

SEQ ID NO: 60 - protein, 10 aa, Canis sp.
KSAVSIDVGV

SEQ ID NO: 61 - protein, 5 aa, Canis sp.
TYVMN

SEQ ID NO: 62 - protein, 17 aa, Canis sp.
SINGGGSSPTYADAVRG

SEQ ID NO: 63 - protein, 8 aa, Canis sp.
SMVGPFDY

SEQ ID NO: 64 - protein, 11 aa, Canis sp.
SGKSLSYYYAQ

SEQ ID NO: 65 - protein, 7 aa, Canis sp.
KDTERPS

SEQ ID NO: 66 - protein, 10 aa, Canis sp.
ESAVSSDTIV

SEQ ID NO: 67 - protein, 122 aa, Mus musculus
QVQLQQSAAELARPGASVKMSCKTSGYTFTSYTIHWIKQRPGQGLEWIGNINPTSGYTENNQRFKDKTTLTVDRSSNTAYLQLHSLTSEDSAVYFCARWGFKYDGEWSFDVWGAGTTVTVSS

SEQ ID NO: 68 - DNA, 366 bp, Mus musculus
  source 1..366: mol_type = other DNA
caggtccagctgcagcagtctgcagctgaactggcaagacctggggcctcagtgaagatgtcctgcaagacttctggctacacatttacttcctacacgatacactggataaaacagaggcctggacagggtctggaatggattggaaacattaatcccaccagtggatacactgagaacaatcagaggttcaaggacaagaccacattgactgtagacagatcctccaacacagcctatttgcaactgcacagcctgacatctgaggactctgcggtctatttctgtgcaagatggggctttaaatatgacggagaatggtccttcgatgtctggggcgcagggaccacggtcaccgtctcctca

SEQ ID NO: 69 - protein, 107 aa, Mus musculus
DIQMNQSPSSLSASLGDTITVTCRASQGISIWLSWYQQKPGNIPKVLINKASNLHIGVPPRFSGSGSGTHFTLTITSLQPEDIATYYCLQSQTYPLTFGGGTKLEIN

SEQ ID NO: 70 - DNA, 321 bp, Mus musculus
  source 1..321: mol_type = other DNA
gacatccaaatgaaccagtctccatccagtctgtctgcatccctcggagacacaatcaccgtcacttgccgtgccagtcagggcatcagtatttggttaagctggtaccagcagaaaccaggaaatattcctaaagtattgatcaataaggcttccaacttgcacataggagtcccaccaaggtttagtggcagtggatctggaacacatttcacattaactatcaccagcctacagcctgaagacattgccacttactactgtctacagagtcaaacttatcctctcacgttcggaggggggaccaagctggaaataaac

SEQ ID NO: 71 - protein, 121 aa, Mus musculus
QVQLQQSGAELVKPGASVKLSCKASGYTFKYYDINWVRQRPEQGLEWIGWIFPGDGGTKYNETFKGKATLTTDKSSSTAYMQLSRLTSEDSAVYFCARGGTSVIRDAMDYWGQGTSVTVSS

SEQ ID NO: 72 - DNA, 363 bp, Mus musculus
  source 1..363: mol_type = other DNA
caggttcagctgcagcagtctggagctgaactggtaaagcctggggcttcagtgaagttgtcctgcaaggcttctggctacaccttcaaatactatgatataaactgggtgaggcagaggcctgaacagggacttgagtggattggatggatttttcctggagatggtggtactaagtacaatgagacgttcaagggcaaggccacactgactacagacaaatcctccagcacagcctacatgcagctcagcaggctgacatctgaggactctgctgtctatttctgtgcaagaggggggacttcggtgataagggatgctatggactactggggtcaaggaacctcagtcaccgtctcctca

SEQ ID NO: 73 - protein, 111 aa, Mus musculus
DIVLTQSPASLAVSLGQRATISCRASESVDNYGISFMHWYQQKPGQPPKLLIYRASNLESGIPARFSGSGSRTDFTLTINPVETDDVATYYCQQSNKDPLTFGAGTKLELK

SEQ ID NO: 74 - DNA, 333 bp, Mus musculus
  source 1..333: mol_type = other DNA
gacattgtgctgacccaatctccagcttctttggctgtgtctctagggcagagggccaccatctcctgcagagccagcgaaagtgttgataattatggcattagttttatgcactggtaccagcagaaaccaggacagccacccaaactcctcatctatcgtgcatccaacctagaatctgggatccctgccaggttcagtggcagtgggtctaggacagacttcaccctcaccattaatcctgtggagactgatgatgttgcaacctattactgtcagcaaagtaataaggatccgctcacgttcggtgctgggaccaagctggagctgaaa

SEQ ID NO: 75 - protein, 124 aa, Canis sp.
EVQLVESGGDLVKPGGSLRLSCVASGFTFSSYGMSWVRQAPGKGLQWVAHINSGGSSTYYADAVKGRFTISRDNAKNTLYLQMNSLRAEDTAVYYCVEVYTTLAAFWTDNFDYWGQGTLVTVSS

SEQ ID NO: 76 - DNA, 372 bp, Canis sp.
  source 1..372: mol_type = other DNA
gaggtgcagctggtggagtctgggggagacctggtgaagcctggggggtccctgagactctcctgtgtggcttctggattcaccttcagtagttatggcatgagctgggtccgccaggctccagggaaggggctgcagtgggtcgcacacattaacagtggtggaagtagcacatactacgcagacgctgtgaagggacgattcaccatctccagagacaacgccaagaacacgctctatctgcagatgaacagcctgagagctgaggacacggccgtctattactgtgtggaggtttacactacgttagctgcattctggacagacaattttgactactggggccagggaaccctggtcaccgtctcctca

SEQ ID NO: 77 - protein, 110 aa, Canis sp.
QSVLTQPTSVSGSLGQRVTISCSGSTNNIGILAATWYQQLPGKAPKVLVYSDGNRPSGVPDRFSGSKSGNSATLTITGLQAEDEADYYCQSFDTTLDAYVFGSGTQLTVL

SEQ ID NO: 78 - DNA, 330 bp, Canis sp.
  source 1..330: mol_type = other DNA
cagtctgtgctgactcagccgacctcagtgtcggggtcccttggccagagggtcaccatctcctgctctggaagcacgaacaacatcggtattcttgctgcgacctggtaccaacaactcccaggaaaggcccctaaagtcctcgtgtacagtgatgggaatcgaccgtcaggggtccctgaccggttttccggctccaagtctggcaactcagccaccctgaccatcactgggcttcaggctgaggacgaggctgattattactgccagtcctttgataccacgcttgatgcttacgtgttcggctcaggaacccaactgaccgtcctt

SEQ ID NO: 79 - protein, 117 aa, Canis sp.
EVQLVESGGDLVKPAGSLRLSCVASGFTFSDYAMSWVRQAPGRGLQWVAGIDSVGSGTSYADAVKGRFTISRDDAKNTLYLQMFNLRAEDTAIYYCASGFPGSFEHWGQGTLVTVSS

SEQ ID NO: 80 - DNA, 351 bp, Canis sp.
  source 1..351: mol_type = other DNA
gaggtgcagctggtggagtctgggggagacctggtgaagcctgcagggtccctgagactgtcctgtgtggcctctggattcaccttcagtgactatgccatgagctgggtccgccaggctcctgggaggggactgcagtgggtcgcaggtattgacagtgttggaagtggcacaagctacgcagacgctgtgaagggccgattcacaatctccagagacgacgccaagaacacactgtatctgcagatgttcaacctgagagccgaggacacggccatatattactgtgcgagcgggttccctgggtcctttgagcactggggccagggcaccctggtcaccgtctcctca

SEQ ID NO: 81 - protein, 104 aa, Canis sp.
QSVLTQPASVSGSLGQKVTISCTGSSSNIGSGYVGWYQQLPGTGPRTLIYYNSDRPSGVPDRFSGSRSGTTATLTISGLQAEDEADYYCSVYDRTFNAVFGGGT

SEQ ID NO: 82 - DNA, 327 bp, Canis sp.
  source 1..327: mol_type = other DNA
cagtctgtactgactcagccggcctcagtgtctgggtccctgggccagaaggtcaccatctcctgcactggaagtagttccaacattggtagtggttatgtgggctggtaccagcagctcccaggaacaggccccagaaccctcatctattataacagtgaccgaccttcgggggtccccgatcgattctctggctccaggtcaggcaccacagcaaccctgaccatctctggactccaggctgaggacgaggctgattattactgctcagtatatgacaggactttcaatgctgtgttcggcggaggcacccacctgaccgtcctc

SEQ ID NO: 83 - protein, 118 aa, Canis sp.
EVQLVESGGDLVKPPGSLRLSCVASGFTFSSYDMTWVRQAPGKGLQWVADVNSGGTGTAYAVAVKGRFTISRDNAKKTLYLQMNSLRAEDTAVYYCAKLGVRDGLSVWGQGTLVTVSS

SEQ ID NO: 84 - DNA, 354 bp, Canis sp.
  source 1..354: mol_type = other DNA
gaggtgcagctggtggagtctgggggagacctggtgaagcctccagggtccctgagactgtcctgtgtggcctctggattcaccttcagcagttatgacatgacctgggtccgccaggctcctgggaagggactgcagtgggtcgcagatgttaacagtggtggaactggcacggcctacgcagtcgctgtgaagggccgattcaccatctccagagacaacgccaagaaaacactctatttacagatgaacagcctgagagccgaagacacggccgtttattattgtgcgaaactaggtgtgagagatggtctttctgtctggggccagggcaccctggtcaccgtctcctcg

SEQ ID NO: 85 - protein, 107 aa, Canis sp.
SSVLTQPPSVSVSLGQTATISCSGESLNEYYTQWFQQKAGQAPVLVIYRDTERPSGIPDRFSGSSSGNTHTLTISGARAEDEADYYCESAVDTGTLVFGGGTHLAVL

SEQ ID NO: 86 - DNA, 321 bp, Canis sp.
  source 1..321: mol_type = other DNA
tccagtgtgctgactcagcctccctcggtatcagtgtctctgggacagacagcaaccatctcctgctctggagagagtctgaatgaatattatacacaatggttccagcagaaggcaggccaagcccctgtcttggtcatatatagggacactgagcggccctctgggatccctgaccgattctctggctccagttcagggaacacacacaccctaaccatcagcggggctcgggccgaggacgaggctgactattactgcgagtcagcggtcgacactggaacccttgtctttggcggaggcacccacctggccgtcctc

SEQ ID NO: 87 - protein, 117 aa, Canis sp.
EVQLVESGGDLVKPAGSLRLSCVASGFTFRTYVMNWVRQAPGKGLQWVASINGGGSSPTYADAVRGRFTVSRDNAQNSLFLQMNSLRAEDTAVYFCVVSMVGPFDYWGQGTLVTVSS

SEQ ID NO: 88 - DNA, 351 bp, Canis sp.
  source 1..351: mol_type = other DNA
gaggtgcagctggtggagtctgggggagacctggtgaagcctgcagggtccctgagactgtcctgtgtggcctctggattcaccttcaggacctatgtcatgaactgggtccgccaggctcctgggaaggggctgcaatgggtcgcaagtattaacggtggtggaagtagcccaacctacgcagacgctgtgaggggccgattcaccgtctccagggacaacgcccagaactcactgtttctgcagatgaacagcctgagagccgaggacacagccgtgtatttttgtgtcgtgtcgatggttgggcccttcgactactggggccaagggaccctggtcaccgtgtcctca

SEQ ID NO: 89 - protein, 102 aa, Canis sp.
SSVLTQPPSVSVSLGQTATISCSGESLSNYYAQWFQQKAGQAPVLVIYKDTERPSGIPDRFSGSSSGNTHTLTISGARAEDEADYYCESAVSSDTIVFGGGT

SEQ ID NO: 90 - DNA, 321 bp, Canis sp.
  source 1..321: mol_type = other DNA
tccagtgtgctgactcagcctccctcggtatcagtgtctctggggcagacagcaaccatctcctgctctggagagagtctgagtaactattatgcacaatggttccagcagaaggcaggccaagcccctgtgttggtcatatataaggacactgagcggccctctgggatccctgaccgattctctggctccagttcagggaacacacacaccctgaccatcagcggggctcgggccgaggacgaggctgactattactgtgagtcagcagtcagttctgatactattgtgttcggcggaggcacccacctgaccgtcctc

SEQ ID NO: 91 - protein, 124 aa, Canis sp.
EVQLVESGGDLVKPAGSLRLSCVASGFTFSSYAMKWVRQAPGKGLQWVATINNDGTRTGYADAVRGRFTISKDNAKNTLYLQMDSLRADDTAVYYCTKGNAESGCTGDHCPPYWGQGTLVTVSS

SEQ ID NO: 92 - DNA, 372 bp, Canis sp.
  source 1..372: mol_type = other DNA
gaggtgcagctggtggagtctgggggagacctggtgaagcctgcggggtccctgagactgtcctgtgtggcctctggattcaccttcagtagttatgccatgaaatgggtccgccaggctcctgggaaggggctgcagtgggtcgcgactattaacaatgatggaaccagaacaggctacgcagacgctgtgaggggccgattcaccatctccaaagacaacgccaaaaacacactgtatctgcagatggacagcctgagagccgacgacacggccgtctattactgtacaaagggcaatgccgaatccggctgtactggtgatcactgtcctccctactggggccagggaaccctggtcaccgtctcctca

SEQ ID NO: 93 - protein, 107 aa, Canis sp.
SSVLTQPPSVSVSLGQTATISCSGESLNKYYAQWFQQKAGQAPVLVIYKDTERPSGIPDRFSGSSAGNTHTLTISGARAEDEADYYCESAVSSETNVFGSGTQLTVL

SEQ ID NO: 94 - DNA, 321 bp, Canis sp.
  source 1..321: mol_type = other DNA
tccagtgtgctgactcagcctccctcggtatcagtgtctctgggacagacagcaaccatctcctgctctggagagagtctgaataaatattatgcacaatggttccaacagaaggcaggccaagcccctgtgttggtcatatataaggacactgagcggccctctgggatccctgaccgattctccggctccagtgcaggcaacacacacaccctgaccatcagcggggctcgggccgaggacgaggctgactattactgcgagtcagcagtcagttctgaaactaacgtgttcggctcaggaacccaactgaccgtcctt

SEQ ID NO: 95 - protein, 116 aa, Canis sp.
EVQLVDSGGDLVKPGGSLRLSCVASGFTFSTYFMSWVRQAPGRGLQWVALISSDGSGTYYADAVKGRFTISRDNAKNTLYLQMNSLRAEDTAMYYCAIFWRAFNDWGQGTLVTVSS

SEQ ID NO: 96 - DNA, 348 bp, Canis sp.
  source 1..348: mol_type = other DNA
gaggtacaactggtggactctgggggagacctggtgaagcctggggggtccctgagactctcctgtgtggcctctggattcaccttcagtacctacttcatgtcctgggtccgccaggctccagggagggggcttcagtgggtcgcacttattagcagtgatggaagtggcacatactacgcagacgctgtgaagggccgattcaccatctccagagacaatgccaagaacacgctgtatctgcagatgaacagcctgagagccgaggacacggctatgtattactgtgcgatattctggcgggcctttaacgactggggccagggcaccctggtcaccgtctcctca

SEQ ID NO: 97 - protein, 110 aa, Canis sp.
QTVVIQEPSLSVSPGGTVTLTCGLNSGSVSTSNYPGWYQQTRGRTPRTIIYDTGSRPSGVPNRFSGSISGNKAALTITGAQPEDEADYYCSLYTDSDILVFGGGTHLTVL

SEQ ID NO: 98 - DNA, 330 bp, Canis sp.
  source 1..330: mol_type = other DNA
cagactgtggtaatccaggagccatcactctcagtgtctccaggagggacagtcacactcacatgtggcctcaactctgggtcagtctccacaagtaattaccctggctggtaccagcagacccgaggccggactcctcgcacgattatctacgacacaggcagtcgcccctctggggtccctaatcgcttctccggatccatctctggaaacaaagccgccctcaccatcacaggagcccagcccgaggatgaggctgactattactgttccttatatacggatagtgacattcttgttttcggcggaggcacccacctgaccgtcctc

SEQ ID NO: 99 - protein, 117 aa, Canis sp.
EVHLVESGGDLVKPWGSLRLSCVASGFTFSDRGMSWVRQSPGKGLQWVAYIRYDGSRTDYADAVEGRFTISRDNAKNTLYLQMNSLRAEDTAVYYCARWDGSSFDYWGQGTLVTVSS

SEQ ID NO: 100 - DNA, 351 bp, Canis sp.
  source 1..351: mol_type = other DNA
gaggtgcatttggtggagtctgggggagacctggtgaagccttgggggtccttgagactgtcctgtgtggcctctggattcacctttagtgatcgtggcatgagctgggtccgtcagtctccagggaaggggctgcagtgggtcgcatatattaggtatgatgggagtaggacagactacgcagacgctgtggagggccgattcaccatctccagagacaacgccaagaacacgctctacctgcagatgaacagcctgagagccgaggacacggccgtgtattactgtgcgagatgggacggtagttcttttgactattggggccagggcaccctggtcaccgtctcctca

SEQ ID NO: 101 - protein, 112 aa, Canis sp.
DIVVTQTPLSLSVSPGETASFSCKASQSLLHSDGNTYLDWFRQKPGQSPQRLIYKVSNRDPGVPDRFSGSGSGTDFTLRISGVEADDAGLYYCMQAIHFPLTFGAGTKVELK

SEQ ID NO: 102 - DNA, 336 bp, Canis sp.
  source 1..336: mol_type = other DNA
gatattgtcgtgacacagaccccgctgtccctgtccgtcagccctggagagactgcctccttctcctgcaaggccagtcagagcctcctgcacagtgatggaaacacgtatttggattggttccgacagaagccaggccagtctccacagcgtttgatctacaaggtctccaacagagaccctggggtcccagacaggttcagtggcagcgggtcagggacagatttcaccctgagaatcagcggagtggaggctgacgatgctggactttattactgcatgcaagcaatacactttcctctgacgttcggagcaggaaccaaggtggagctcaaa

SEQ ID NO: 103 - protein, 119 aa, Canis sp.
EVQLVESGGDLVKPAGSLRLSCVASGFTFSSYVMTWVRQAPGKGLQWVAGINSEGSRTAYADAVKGRFTISRDNAKNTLYLQIDSLRAEDTAIYYCATGDIVATGTSYWGQGTLVTVSS

SEQ ID NO: 104 - DNA, 357 bp, Canis sp.
  source 1..357: mol_type = other DNA
gaggtgcagctggtggagtctgggggagacctggtgaagcctgcagggtccctgagactgtcctgtgtggcctctggattcaccttcagtagttatgtcatgacctgggtccgccaggctcctgggaagggactgcagtgggtcgcaggcattaatagtgaggggagtaggacagcctacgcagacgctgtgaagggccgattcaccatctccagagacaacgccaagaatacactttatctacaaatagacagcctgagagccgaggacacggccatatattactgtgcgacaggcgatatagtagcgactggtacttcgtattggggccagggcaccctggtcaccgtctcctca

SEQ ID NO: 105 - protein, 107 aa, Canis sp.
SNVLTQPPSVSVSLGQTATISCSGETLNRFYTQWFQQKAGQAPVLVIYKDTERPSGIPDRFSGSSSGNIHTLTISGARAEDEAAYYCKSAVSIDVGVFGGGTHLTVF

SEQ ID NO: 106 - DNA, 321 bp, Canis sp.
  source 1..321: mol_type = other DNA
tccaatgtactgactcagcctccctcggtatcagtgtctctgggacagacagcaaccatctcctgctctggagagactctgaatagattttatacacaatggttccagcagaaggcaggccaagcccctgtgttggtcatatataaggacactgagcggccctctgggatccctgaccgattctccggctccagttcagggaacatacacaccctgaccatcagcggggctcgggccgaggacgaggctgcctattactgcaagtcagcagtcagtattgatgttggtgtgttcggcggaggcacccacctgaccgtcttc

SEQ ID NO: 107 - protein, 117 aa, Canis sp.
EVQLVESGGDLVKPAGSLRLSCVASGFTFRTYVMNWVRQAPGKGLQWVASINGGGSSPTYADAVRGRFTVSRDNAQNSLFLQMNSLRAEDTAIYFCVVSMVGPFDYWGHGTLVTVSS

SEQ ID NO: 108 - DNA, 351 bp, Canis sp.
  source 1..351: mol_type = other DNA
gaggtgcagctggtggagtctgggggagacctggtgaagcctgcagggtccctgagactgtcctgtgtggcctctggattcaccttcaggacctatgtcatgaactgggtccgccaggctcctgggaaggggctgcaatgggtcgcaagtattaacggtggtggaagtagcccaacctacgcagacgctgtgaggggccgattcaccgtctccagggacaacgcccagaactcactgtttctgcagatgaacagcctgagagccgaggacacagccatatatttttgtgtcgtgtcgatggttgggcccttcgactactggggccatgggaccctggtcaccgtgtcctca

SEQ ID NO: 109 - protein, 107 aa, Canis sp.
SSVLTQPPSVSVSLGQTATISCSGKSLSYYYAQWFQQKAGQAPVLVIYKDTERPSGIPDRFSGSSSGNTHTLTISGARAEDEADYYCESAVSSDTIVFGGGTHLTVL

SEQ ID NO: 110 - DNA, 321 bp, Canis sp.
  source 1..321: mol_type = other DNA
tccagtgtgctgactcagcctccctcggtatcagtgtctctggggcagacagcaaccatctcctgctctggaaagagtctgagttactattatgcacaatggttccagcagaaggcaggccaagcccctgtgttggtcatatataaggacactgagcggccctctgggatccctgaccgattctctggctccagttcagggaacacacacaccctgaccatcagcggggctcgggccgaggacgaggctgactattactgtgagtcagcagtcagttctgatactattgtgttcggcggaggcacccacctgaccgtcctc

SEQ ID NO: 111 - protein, 121 aa, synthetic construct
  REGION 1..121: note = Felinized variable heavy mAb sequence, from Mus musculus and Felis catus
QVLLVQSGAEVKKPGASVKIFCKASGYSFTYYDINWLRQAPEQGLEWMGWIFPGDGGTKYNETFKGRLTLTADTSTNTVYMELSSLRSADTAMYYCARGGTSVIRDAMDYWGQGALVTVSS

SEQ ID NO: 112 - DNA, 363 bp, synthetic construct
  misc_feature 1..363: note = Nucleotide sequence encoding felinized variable heavy mAb sequence, from Mus musculus and Felis catus
  source 1..363: mol_type = other DNA
caggtgctgctggtccagtcaggagcagaggtaaaaaagcccggggcgagtgtcaagattttctgtaaggcctccggatactcttttacgtattacgatattaactggcttcgccaggcccctgagcaggggctcgaatggatgggttggatattccccggagatgggggaaccaagtacaacgaaaccttcaaggggaggctgaccctgactgcagataccagcacgaacacagtgtatatggagttgtcctcactgcgatctgctgatactgccatgtactactgcgctcgcggcggcacttcagttatcagggatgccatggactattgggggcagggcgcactcgtcactgtctcgagc

SEQ ID NO: 113 - protein, 106 aa, synthetic construct
  REGION 1..106: note = Felinized variable light mAb sequence, from Mus musculus and Felis catus
EIQMTQSPSSLSASPGDRVTITCRASESVDNYGISFMHWYQQKPGKVPKLLIYRASNLESGVPSRFSGSGSGTDFTLTISSLEPEDAATYYCQQSNKDPLTFGQGT

SEQ ID NO: 114 - DNA, 333 bp, synthetic construct
  misc_feature 1..333: note = Nucleotide sequence encoding felinized variable light mAb sequence, from Mus musculus and Felis catus
  source 1..333: mol_type = other DNA
gaaatccagatgacacaatctcccagctccctcagcgcatctcctggcgacagggtaaccatcacctgccgcgccagcgagtcagtagacaactatggcatatccttcatgcactggtatcaacaaaagcccgggaaagtccccaaactgttgatttacagagcaagcaatctcgagtcaggagtcccatctcgcttctctggttccggttccggaaccgacttcactctgacaatttcttctctggagcccgaggatgccgctacatattactgtcagcaaagcaataaagatccactgaccttcggacagggtaccaagctggagatcaaa

SEQ ID NO: 115 - protein, 106 aa, synthetic construct
  REGION 1..106: note = Felinized variable light mAb sequence, from Mus musculus and Felis catus
EVVLTQSSAFLSRTLKEKATITCRASESVDNYGISFMHWYQQKPNQAPKLLVKRASNLESGAPSRFSGSGSGTDFTLTISSPEPEDAATYYCQQSNKDPLTFGQGT

SEQ ID NO: 116 - DNA, 318 bp, synthetic construct
  misc_feature 1..318: note = Nucleotide sequence encoding felinized variable light mAb sequence, from Mus musculus and Felis catus
  source 1..318: mol_type = other DNA
gaggtggtgctgactcagagtagcgcgtttctgtctcggaccctgaaagagaaagctaccatcacgtgcagggcaagcgagagcgtggacaactatggtatcagcttcatgcattggtatcagcagaaacctaatcaggcgcctaagctgctcgtgaaaagagcctccaaccttgagagcggcgcaccatcaaggttttcaggaagtggcagcgggacagacttcacccttacaatctctagtccagagccggaggacgcagctacctactattgccagcaatccaataaagacccgttgacattcggccaaggtacc

SEQ ID NO: 117 - protein, 106 aa, synthetic construct
  REGION 1..106: note = Felinized variable light mAb sequence, from Mus musculus and Felis catus
EIQMTQSPSSLSASPGDRVTITCRASESVDNYGISFMHWYQQKPGQPPKLLIYRASNLESGVPSRFSGSGSGTDFTLTISSLEPEDAATYYCQQSNKDPLTFGQGT

SEQ ID NO: 118 - DNA, 318 bp, synthetic construct
  misc_feature 1..318: note = Nucleotide sequence encoding felinized variable light mAb sequence, from Mus musculus and Felis catus
  source 1..318: mol_type = other DNA
gagatccagatgacccagtctccatcctcactgagtgctagcccgggggatcgagtgactataacatgtcgggccagtgaatcagtggacaactatggaatcagttttatgcactggtatcagcagaagcccggccagccaccgaagctgttgatttatcgcgcaagcaatctggagtcaggagtgccctctagattttctgggagcggttctggcacagatttcacactcacaatatcatccttggaaccggaagacgcagccacatactattgccagcagagtaacaaggaccctttgacttttggccagggtacc

SEQ ID NO: 119 - protein, 106 aa, synthetic construct
  REGION 1..106: note = Felinized variable light mAb sequence, from Mus musculus and Felis catus
EIQMTQSPSSLSASPGDRVTITCRASESVDNYGISFMHWYQQKPGQVPKLLIYRASNLESGVPSRFSGSGSGTDFTLTISSLEPEDAATYYCQQSNKDPLTFGQGT

SEQ ID NO: 120 - DNA, 318 bp, synthetic construct
  misc_feature 1..318: note = Nucleotide sequence encoding felinized variable light mAb sequence, from Mus musculus and Felis catus
  source 1..318: mol_type = other DNA
gagatccagatgacccagtctccatcctcactgagtgctagcccgggggatcgagtgactataacatgtcgggccagtgaatcagtggacaactatggaatcagttttatgcactggtatcagcagaagcccggccaggtcccgaagctgttgatttatcgcgcaagcaatctggagtcaggagtgccctctagattttctgggagcggttctggcacagatttcacactcacaatatcatccttggaaccggaagacgcagccacatactattgccagcagagtaacaaggaccctttgacttttggccagggtacc

SEQ ID NO: 121 - protein, 122 aa, synthetic construct
  REGION 1..122: note = Felinized variable heavy mAb sequence, from Mus musculus and Felis catus
QVLLVQSGAEVRTPGASVKIFCKASGYSFTSYTIHWLRQAPAQGLEWMGNINPTSGYTENNQRFKDRLTLTADTSTNTAYMELSSLRSADTAMYYCARWGFKYDGEWSFDVWGAGTTVTVSS

SEQ ID NO: 122 - DNA, 366 bp, synthetic construct
  misc_feature 1..366: note = Nucleotide sequence encoding felinized variable heavy mAb sequence, from Mus musculus and Felis catus
  source 1..366: mol_type = other DNA
caagtgcttctggtgcaaagcggggcggaagttaggaccccaggagcctcagtaaaaattttttgtaaggcatccggctacagtttcaccagctacactattcactggctgaggcaggccccggcccaagggctggagtggatgggaaatatcaatcccacgtctggctatacagagaataaccaaaggtttaaggataggctgactctgacagctgacacatcaaccaatacggcatacatggagctctcctctctccggagtgccgacaccgccatgtactactgtgctcggtgggggtttaaatacgatggcgagtggagcttcgacgtgtggggcgcgggcacaaccgtgaccgtctcgagc

SEQ ID NO: 123 - protein, 122 aa, synthetic construct
  REGION 1..122: note = Felinized variable heavy mAb sequence, from Mus musculus and Felis catus
QVLLVQSGAEVRTPGASVKIFCKASGYGFTSYTIHWVRQSPAQGLEWMGNINPTSGYTENNQRFKDRLTLTADTSTNTAYMELSSLRSADTAMYYCARWGFKYDGEWSFDVWGAGTTVTVSS

SEQ ID NO: 124 - DNA, 366 bp, synthetic construct
  misc_feature 1..366: note = Nucleotide sequence encoding felinized variable heavy mAb sequence, from Mus musculus and Felis catus
  source 1..366: mol_type = other DNA
caggtgctgctcgtgcagagcggagccgaagtgaggacacccggtgcgagtgtaaaaattttttgcaaggcaagcggctacgggtttacatcctataccatccactgggtgaggcagtccccagcgcagggacttgaatggatgggaaatattaatccaacaagcgggtatactgaaaacaaccaaagatttaaggacagactgacactcaccgcagatacatctacaaatacagcctacatggagttgtcttccctgcggagtgccgacacggctatgtactactgtgctcggtgggggtttaagtatgatggcgaatggtccttcgacgtctggggagctggaaccaccgtgaccgtctcgagc

SEQ ID NO: 125 - protein, 122 aa, synthetic construct
  REGION 1..122: note = Felinized variable heavy mAb sequence, from Mus musculus and Felis catus
QVLLVQSGAEVRKPGASVKIFCKASGYSFTSYTIHWLRQAPEQGLEWMGNINPTSGYTENNQRFKDRLTLTADTSTNTAYMELSSLRSADTAMYYCARWGFKYDGEWSFDVWGAGTTVTVSS

SEQ ID NO: 126 - DNA, 366 bp, synthetic construct
  misc_feature 1..366: note = Nucleotide sequence encoding felinized variable heavy mAb sequence, from Mus musculus and Felis catus
  source 1..366: mol_type = other DNA
caggtcctcttggttcaaagcggagccgaagtccgaaaaccgggtgcctcagtgaaaatcttctgtaaggcctccggctatagtttcacgagttacacaatccactggctgcgacaggcaccagagcagggactggagtggatgggaaatataaatccgacgtctgggtacacagaaaacaaccagagattcaaggatagattgacactgaccgcggatactagtacaaatacggcttacatggaactgtcctcactccggtcagccgacaccgccatgtattactgtgctcgctgggggttcaagtatgatggagagtggagcttcgacgtatggggagccggaaccactgtgaccgtctcgagc

SEQ ID NO: 127 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGKVPKLLIYKASNLHIGVPSRFSGSGSGTDFTLTISSLEPEDAATYYCLQSQTYPLTFGGGTKLEIK

SEQ ID NO: 128 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattcagatgactcaatcaccttcatctttgagtgcatcacccggagatagagttacaatcacctgcagggcgagtcaagggatctccatatggttgtcatggtatcagcagaaacccggaaaggtcccgaaactcttgatctacaaggcctctaacctgcacattggcgtgccaagccgattcagcgggagcggaagtggcaccgactttactctgacgatcagttcactggagcccgaggacgctgcaacatactattgtctgcaatctcagacctaccctctcacctttggaggaggtaccaagctggagatcaaa

SEQ ID NO: 129 - protein, 107 aa, synthetic construct
  REGION 1..107: note = Felinized variable light mAb sequence, from Mus musculus and Felis catus
EITMTQSPGSLAGSPGQQVTMNCRASQGISIWLSWYQQKPGQHPKLLIYKASNLHIGVPDRFSGSGSGTDFTLTISNLQAEDVASYYCLQSQTYPLTFGGGTKLEIK

SEQ ID NO: 130 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattaccatgacacaaagccccggctccctggccggctcccccggacagcaagtgaccatgaattgtcgggccagccagggaatttctatatggctctcttggtatcagcaaaaacccggacagcaccctaaacttctgatctacaaagcaagtaacttgcacatcggcgtccctgatcgattcagtggctcaggttccggtacagattttactcttaccatcagcaatctgcaggctgaggatgtggcaagctattactgtctccaaagtcagacttaccctctgacatttgggggcggtaccaagctggagatcaaa

SEQ ID NO: 131 - protein, 107 aa, synthetic construct
  REGION 1..107: note = Felinized variable light mAb sequence, from Mus musculus and Felis catus
EVVLTQSSAFLSRTLKEKATITCRASQGISIWLSWYQQKPNQAPKLLVKKASNLHIGAPSRFSGSGSGTDFTLTISSPEPEDAATYYCLQSQTYPLTFGGGTKLGDQ

SEQ ID NO: 132 - DNA, 322 bp, synthetic construct
  misc_feature 1..322: note = Nucleotide sequence encoding felinized variable light mAb sequence, from Mus musculus and Felis catus
  source 1..322: mol_type = other DNA
gaggtagtgctgactcagtcctccgccttcttgtcaagaactctcaaagagaaagcaacaatcacttgtcgggcgtctcaagggatatcaatttggctgagctggtatcagcagaaaccaaatcaagcgccgaaactgctggtgaagaaggcctccaatctccacattggcgcacccagcaggttttccggcagtggctctggcacagatttcactctgaccatcagctcacccgagcccgaagacgccgctacatactattgcttgcaatcccagacataccccctgacttttgggggaggtaccaagctgggagatcaaa

SEQ ID NO: 133 - protein, 107 aa, synthetic construct
  REGION 1..107: note = Felinized variable light mAb sequence, from Mus musculus and Felis catus
DIQMNQSPSSLSASLGDTITVTCRASQGISIWLSWYQQKPGKVPKLLIYKASNLHIGVPSRFSGSGSGTDFTLTISSLEPEDAATYYCLQSQTYPLTFGGGTKLEIK

SEQ ID NO: 134 - DNA, 321 bp, synthetic construct
  misc_feature 1..321: note = Nucleotide sequence encoding felinized variable light mAb sequence, from Mus musculus and Felis catus
  source 1..321: mol_type = other DNA
gacattcagatgaatcagtctcctagctcactgtcagccagccttggagacaccattacagtcacttgcagggcgagtcaagggatctccatatggttgtcatggtatcagcagaaacccggaaaggtcccgaaactcttgatctacaaggcctctaacctgcacattggcgtgccaagccgattcagcgggagcggaagtggcaccgactttactctgacgatcagttcactggagcccgaggacgctgcaacatactattgtctgcaatctcagacctaccctctcacctttggaggaggtaccaagctggagatcaaa

SEQ ID NO: 135 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGNIPKVLINKASNLHIGVPSRFSGSGSGTDFTLTISSLEPEDAATYYCLQSQTYPLTFGGGTKLEIK

SEQ ID NO: 136 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattcagatgactcaatcaccttcatctttgagtgcatcacccggagatagagttacaatcacctgcagggcgagtcaagggatctccatatggttgtcatggtatcagcagaaaccgggcaatatcccaaaggtgctgattaacaaggcctctaacctgcacattggcgtgccaagccgattcagcgggagcggaagtggcaccgactttactctgacgatcagttcactggagcccgaggacgctgcaacatactattgtctgcaatctcagacctaccctctcacctttggaggaggtaccaagctggagatcaaa

SEQ ID NO: 137 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGKVPKLLIYKASNLHIGVPPRFSGSGSGTHFTLTITSLQPEDIATYYCLQSQTYPLTFGGGTKLEIK

SEQ ID NO: 138 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattcagatgactcaatcaccttcatctttgagtgcatcacccggagatagagttacaatcacctgcagggcgagtcaagggatctccatatggttgtcatggtatcagcagaaacccggaaaggtcccgaaactcttgatctacaaggcctctaacctgcacattggggtccccccaaggttcagcggatctggatccgggacccactttactctgaccataacaagcctgcagcctgaagacattgctacctattactgcctgcaatctcagacctaccctctcacctttggaggaggtaccaagctggagatcaaa

SEQ ID NO: 139 - protein, 107 aa, synthetic construct
  REGION 1..107: note = Felinized variable light mAb sequence, from Mus musculus and Felis catus
DIQMNQSPSSLSASLGDTITVTCRASQGISIWLSWYQQKPGNIPKVLINKASNLHIGVPSRFSGSGSGTDFTLTISSLEPEDAATYYCLQSQTYPLTFGGGTKLEIK

SEQ ID NO: 140 - DNA, 321 bp, synthetic construct
  misc_feature 1..321: note = Nucleotide sequence encoding felinized variable light mAb sequence, from Mus musculus and Felis catus
  source 1..321: mol_type = other DNA
gacattcagatgaatcagtctcctagctcactgtcagccagccttggagacaccattacagtcacttgcagggcgagtcaagggatctccatatggttgtcatggtatcagcagaaaccgggcaatatcccaaaggtgctgattaacaaggcctctaacctgcacattggcgtgccaagccgattcagcgggagcggaagtggcaccgactttactctgacgatcagttcactggagcccgaggacgctgcaacatactattgtctgcaatctcagacctaccctctcacctttggaggaggtaccaagctggagatcaaa

SEQ ID NO: 141 - protein, 107 aa, synthetic construct
  REGION 1..107: note = Felinized variable light mAb sequence, from Mus musculus and Felis catus
DIQMNQSPSSLSASLGDTITVTCRASQGISIWLSWYQQKPGKVPKLLIYKASNLHIGVPPRFSGSGSGTHFTLTITSLQPEDIATYYCLQSQTYPLTFGGGTKLEIK

SEQ ID NO: 142 - DNA, 321 bp, synthetic construct
  misc_feature 1..321: note = Nucleotide sequence encoding felinized variable light mAb sequence, from Mus musculus and Felis catus
  source 1..321: mol_type = other DNA
gacattcagatgaatcagtctcctagctcactgtcagccagccttggagacaccattacagtcacttgcagggcgagtcaagggatctccatatggttgtcatggtatcagcagaaacccggaaaggtcccgaaactcttgatctacaaggcctctaacctgcacattggggtccccccaaggttcagcggatctggatccgggacccactttactctgaccataacaagcctgcagcctgaagacattgctacctattactgcctgcaatctcagacctaccctctcacctttggaggaggtaccaagctggagatcaaa

SEQ ID NO: 143 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGNIPKVLINKASNLHIGVPPRFSGSGSGTHFTLTITSLQPEDIATYYCLQSQTYPLTFGGGTKLEIK

SEQ ID NO: 144 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattcagatgactcaatcaccttcatctttgagtgcatcacccggagatagagttacaatcacctgcagggcgagtcaagggatctccatatggttgtcatggtatcagcagaaaccgggcaatatcccaaaggtgctgattaacaaggcctctaacctgcacattggggtccccccaaggttcagcggatctggatccgggacccactttactctgaccataacaagcctgcagcctgaagacattgctacctattactgcctgcaatctcagacctaccctctcacctttggaggaggtaccaagctggagatcaaa

SEQ ID NO: 145 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGNVPKLLIYKASNLHIGVPSRFSGSGSGTDFTLTISSLEPEDAATYYCLQSQTYPLTFGGGTKLEIK

SEQ ID NO: 146 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaatccagatgacacagtcccccagtagcctttccgcttcaccgggcgatagagtcactattacgtgcagggcctcccagggtatttctatctggctgagctggtatcagcagaagcccggtaatgtgccaaagctcttgatctacaaggcatctaaccttcatatcggagtgccctcaagatttagtgggtcaggcagcggaaccgatttcacattgaccattagttctctggaaccagaggacgctgccacttactactgcctgcagtcccaaacataccctttgacttttggggggggtaccaagctggagatcaaa

SEQ ID NO: 147 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGKIPKLLIYKASNLHIGVPSRFSGSGSGTDFTLTISSLEPEDAATYYCLQSQTYPLTFGGGTKLEIK

SEQ ID NO: 148 - DNA, 321 bp, synthetic construct
  misc_feature 1..321: note = Nucleotide sequence encoding felinized variable light mAb sequence, from Mus musculus and Felis catus
  source 1..321: mol_type = other DNA
gagattcagatgacccagagcccatcaagcctctccgcttcccccggagaccgggtgaccatcacatgcagagcttcacagggaatctcaatctggctcagctggtatcagcagaagccaggcaagattccgaagttgcttatctataaggccagtaacctgcatatcggagttccatcaagattcagtggtagcggaagtgggacagatttcactctcaccatcagctccctcgaaccagaggacgctgcaacttactactgcctgcagtcccagacatatccacttactttcggcgggggtaccaagctggagatcaaa

SEQ ID NO: 149 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGKVPKVLIYKASNLHIGVPSRFSGSGSGTDFTLTISSLEPEDAATYYCLQSQTYPLTFGGGTKLEIK

SEQ ID NO: 150 - DNA, 322 bp, synthetic construct
  misc_feature 1..322: note = Nucleotide sequence encoding felinized variable light mAb sequence, from Mus musculus and Felis catus
  source 1..322: mol_type = other DNA
gagattcagatgactcagagcccatctagtctctctgcatctcccggagacagagttacgatcacctgcagggctagccaagggatatcaatttggctgtcctggtatcagcaaaaacctggcaaagtgccaaaggtcttgatttacaaagcatccaatttgcacatcggcgtccctagtcgcttttccgggtctggtagcggcaccgacttcaccctcaccataagctcactcgagccggaagatgccgctacttactattgcctgcagtctcagacttaccccctgactttcggcggaggtaccaagctggagatcaaac

SEQ ID NO: 151 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGKVPKLLINKASNLHIGVPSRFSGSGSGTDFTLTISSLEPEDAATYYCLQSQTYPLTFGGGTKLEIK

SEQ ID NO: 152 - DNA, 321 bp, synthetic construct
  misc_feature 1..321: note = Nucleotide sequence encoding felinized variable light mAb sequence, from Mus musculus and Felis catus
  source 1..321: mol_type = other DNA
gagatccagatgacgcagagccctagcagcctctctgcatccccaggagacagagtaacaattacctgtcgcgccagccagggaatatctatatggctgtcatggtatcaacagaaaccgggaaaggttccaaagctcttgatcaataaggctagcaatctgcatattggagtgccctcccgcttctctggtagcggaagtggcacagatttcaccctgaccattagtagtctggagcctgaggatgcggccacctactactgcctccagtcccaaacctatcccctgaccttcggaggaggtaccaagctggagatcaaa

SEQ ID NO: 153 - protein, 107 aa, synthetic construct
  REGION 1..107: note = Felinized variable light mAb sequence, from Mus musculus and Felis catus
EIQMTQSPSSLSASPGDRVTITCRASQGISIWLSWYQQKPGNIPKLLIYKASNLHIGVPSRFSGSGSGTDFTLTISSLEPEDAATYYCLQSQTYPLTFGGGTKLEIK

SEQ ID NO: 154 - DNA, 321 bp, synthetic construct
  misc_feature 1..321: note = Nucleotide sequence encoding felinized variable light mAb sequence, from Mus musculus and Felis catus
  source 1..321: mol_type = other DNA
gaaattcagatgactcagagtcctagcagcctgtccgcaagcccaggtgaccgagtcaccataacctgcagggccagtcaggggatctccatatggctctcttggtatcaacagaaacccggcaatatccctaagctcctgatttataaagcgtcaaatctgcatatcggggtgccatcaagattctctgggtccggctcaggaaccgactttaccctgaccatttcttctctcgaacccgaggatgccgccacctattattgccttcaaagccagacatacccattgaccttcggcggcggtaccaagctggagatcaaa

SEQ ID NO: 155 - protein, 159 aa, Canis sp.
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 156 - DNA, 477 bp, Canis sp.
  source 1..477: mol_type = other DNA
atgctctcccacacaggaccatccaggtttgccctgttcctgctctgctctatggaaaccttgctgtcctcccatatggcacccacccatcagctaccaccaagtgatgtacgaaaaatcatcttggaattacagcccttgtcgaggggacttttggaagactatcagaagaaagagacaggggtgccagaatccaaccgtaccttgctgctgtgtctcacctctgattcccaaccaccacgcctcaacagctcagccatcttgccttatttcagggcaatcagaccattatcagataagaacattattgataaaatcatagaacagcttgacaaactcaaatttcaacatgaaccagaaacagaaatttctgtgcctgcagatacttttgaatgtaaaagcttcatcttgacgattttacagcagttctcggcgtgcctggaaagtgtgtttaagtcactaaactctggacctcag

SEQ ID NO: 157 - protein, 165 aa, synthetic construct
  REGION 1..165: note = Amino acid sequence representing wild-type feline IL-31 with C-terminal His tag
MLSHAGPARFALFLLCCMETLLPSHMAPAHRLQPSDIRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQHHHHHH

SEQ ID NO: 158 - DNA, 495 bp, synthetic construct
  misc_feature 1..495: note = Nucleotide sequence representing wild-type feline IL-31 gene with encoded C-terminal His tag
  source 1..495: mol_type = other DNA
atgctttcacacgctggaccagcccgattcgccctcttcctcctctgctgtatggagactctgttgccgtcccacatggccccggcacataggctgcagccgtctgacatccggaagatcattctcgaacttcgccccatgtcgaaggggttgctgcaagactacctgaagaaggagatcggcctgcccgaaagcaaccactcctcgctgccttgcctgtcaagcgattcccagctgccccacattaacggttccgccatcctcccgtacttccgggccatcagaccactgtcggacaagaacaccatcgacaagatcattgaacagctggacaagctgaagtttcagcgcgagcctgaagccaaagtgtccatgcccgccgataacttcgagcggaagaatttcattctcgcggtgctgcagcagttctccgcgtgcctggagcacgtcctgcaatccctgaacagcggacctcagcaccaccatcaccaccat

SEQ ID NO: 159 - protein, 148 aa, synthetic construct
  REGION 1..148: note = Amino acid sequence representing feline IL-31 protein with N-terminal His tag
MRGSHHHHHHGSSHMAPAHRLQPSDIRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 160 - DNA, 444 bp, synthetic construct
  misc_feature 1..444: note = Nucleotide sequence representing feline IL-31 gene with encoded N-terminal His tag
  source 1..444: mol_type = other DNA
atgagaggatcccatcaccatcaccaccacggctcatctcatatggcccccgcacatcgcctgcagccgagtgacattcgtaaaattatcttggagctgcgcccgatgtccaagggcttactgcaggattatctgaagaaagagatcgggctgcctgaaagcaaccatagtagcctgccgtgtttatcgtctgatagccagttaccacacatcaatggctctgcgattttgccctactttcgcgccatccgtccgctgtccgataaaaataccatcgacaaaattatcgaacaactggataaattgaagtttcagcgcgagcctgaagcgaaagtttcgatgccagcmgataacttcgaacgcaaaaactttattttagcggtgttgcagcagttttctgcctgtctggaacacgtgctccagtcactcaatagtgggccacaa

SEQ ID NO: 161 - protein, 165 aa, synthetic construct
  REGION 1..165: note = Amino acid sequence representing mutant Feline IL-31 11E12 protein with C-terminal His tag
MLSHAGPARFALFLLCCMETLLPSHMAPAHRLQPSDIRKIILELRPMSKGLLQDYLKKEIGLPESNHSSLPCLSSDSQLPHINGSAILPYFRAIRPLSDKNTIAKIAEQLDKLKFQREPEAKVSMPADNFERKNFILAVLQQFSACLEHVLQSLNSGPQHHHHHH

SEQ ID NO: 162 - DNA, 495 bp, synthetic construct
  misc_feature 1..495: note = Nucleotide sequence representing mutant feline IL-31 11E12 gene with encoded C-terminal His tag
  source 1..495: mol_type = other DNA
atgctctctcacgccggtcctgcccggttcgcactgttcctcctctgttgcatggagactctgcttccctcccacatggcaccggcccatagactgcagccgtccgacatcagaaagatcatccttgaattgcgccctatgagcaaggggctgctgcaggattacctgaaaaaggagatcggcctgccggaatcgaaccacagctcactgccatgcctgtcctccgactcgcaactgccccacatcaatggatccgccattctgccgtacttccgcgctattcggcctctctccgacaagaacaccatcgccaagattgccgagcagctggataagctgaagttccagagggagccagaagccaaggtgtccatgcccgctgacaacttcgagcggaagaactttatcctcgcggtgctgcagcagttctcagcgtgcctcgaacacgtcttgcaaagcctgaactcgggaccccagcaccaccaccatcatcac

SEQ ID NO: 163 - protein, 165 aa, synthetic construct
  REGION 1..165: note = Amino acid sequence representing mutant feline IL-31 15H05 protein with C-terminal His tag
MLSHAGPARFALFLLCCMETLLPSHMAPAHRLQPSDIRKIILELRPMSKGLLQDYLKKEIGLPESNHSSLPCLSSDSQLPHINGSAILPYFRAIRPLSDKNTIDKIIEQLDKLKFQREPEAKVSMAAANFERKNFILAVLQQFSACLEHVLQSLNSGPQHHHHHH

SEQ ID NO: 164 - DNA, 495 bp, synthetic construct
  misc_feature 1..495: note = Nucleotide sequence representing mutant feline IL-31 15H05 gene with encoded C-terminal His tag
  source 1..495: mol_type = other DNA
atgctctctcacgccggtcctgcccggttcgcactgttcctcctctgttgcatggagactctgcttccctcccacatggcaccggcccatagactgcagccgtccgacatcagaaagatcatccttgaattgcgccctatgagcaaggggctgctgcaggattacctgaaaaaggagatcggcctgccggaatcgaaccacagctcactgccatgcctgtcctccgactcgcaactgccccacatcaatggatccgccattctgccgtacttccgcgctattcggcctctctccgacaagaacaccatcgacaagattattgagcagctggataagctgaagttccagagggagccagaagccaaggtgtccatggccgctgccaacttcgagcggaagaactttatcctcgcggtgctgcagcagttctcagcgtgcctcgaacacgtcttgcaaagcctgaactcgggaccccagcaccaccaccatcatcac

SEQ ID NO: 165 - protein, 152 aa, synthetic construct
  REGION 1..152: note = Amino acid sequence of Equine IL-31 protein with C-terminal His tag
MGWSCIILFLVATATGVHSGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQHHHHHH

SEQ ID NO: 166 - DNA, 456 bp, synthetic construct
  misc_feature 1..456: note = Nucleotide sequence of Equine IL-31 gene with encoded C-terminal His tag
  source 1..456: mol_type = other DNA
atgggctggtcctgcatcattctgtttctggtggccacagccaccggcgtgcactctggacctatctatcagctgcagcccaaagagatccaggccatcatcgtggaactgcagaacctgagcaagaagctgctggacgactacctgaacaaagaaaagggcgtgcagaagttcgacagcgacctgcctagctgcttcaccagcgattctcaggcccctggcaacatcaacagcagcgccatcctgccttacttcaaggccatctctcccagcctgaacaacgacaagagcctgtacatcatcgagcagctggacaagctgaacttccagaacgcccctgaaaccgaggtgtccatgcctaccgacaacttcgagcggaagcggttcatcctgaccatcctgcggtggttcagcaactgcctggaacacagagcccagcaccaccaccatcaccat

SEQ ID NO: 167 - protein, 652 aa, synthetic construct
  REGION 1..652: note = Amino acid sequence of extracellular domain of feline OSMR fused to human IgG1 Fc
MALFSAFQTTFLLALLSLKTYQSEVLSEPLSLAPESLEVSIDSARQCLHLKWSVHNLAYHQELKMVFQIEISRIKTSNVIWVENYSTTVKRNQVLRWSWESKLPLECAKHSVRMRGAVDDAQVPELRFWSNWTSWEEVDVQSSLGHDPLFVFPKDKLVEEGSNVTICYVSRSHQNNISCYLEGVRMHGEQLDPNVCVFHLKNVPFIRETGTNIYCKADQGDVIKGIVLFVSKVFEEPKDFSCETRDLKTLNCTWAPGSDAGLLTQLSQSYTLFESFSGKKTLCKHKSWCNWQVSPDSQEMYNFTLTAENYLRKRSVHLLFNLTHRVHPMAPFNVFVKNVSATNATMTWKVHSIGNYSTLLCQIELDGEGKVIQKQNVSVKVNGKHLMKKLEPSTEYAAQVRCANANHFWKWSEWTRRNFTTAEAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - DNA, 2076 bp, synthetic construct
  misc_feature 1..2076: note = Nucleotide sequence encoding extracellular domain of feline OSMR fused to human IgG1 Fc
  source 1..2076: mol_type = other DNA
atggccctgttcagcgccttccagaccaccttcctgctggccctgctgagcctgaaaacctaccagagcgaggtgctgagcgagcccctgtctctggcccctgagagcctggaagtgtccatcgacagcgccagacagtgcctgcacctgaagtggagcgtgcacaacctggcctaccaccaggaactgaagatggtgttccagatcgagatcagccggatcaagaccagcaacgtgatctgggtggaaaactacagcaccaccgtgaagcggaaccaggtgctgcggtggtcctgggagtctaagctgcctctggaatgcgccaagcacagcgtgcggatgagaggcgccgtggatgatgcccaggtgcccgagctgagattctggtccaactggacctcctgggaagaggtggacgtgcagtctagcctgggccacgaccccctgttcgtgttccccaaggacaagctggtggaagagggctccaacgtgaccatctgctacgtgtccagaagccaccagaacaacatcagctgctacctggaaggcgtgcgcatgcacggcgagcagctggaccctaacgtgtgcgtgttccacctgaagaacgtgcccttcatcagagagacaggcaccaacatctactgcaaggccgaccagggcgacgtgatcaagggcatcgtgctgtttgtgtccaaggtgttcgaggaacccaaggacttcagctgcgagacacgggatctgaaaaccctgaactgtacctgggcccctggctccgatgccggactgctgactcagctgtcccagagctacaccctgttcgagagcttcagcggcaaaaagaccctgtgcaagcacaagagctggtgcaactggcaagtgtcccccgatagccaggaaatgtacaacttcaccctgaccgccgagaactacctgcggaagagatccgtgcatctgctgttcaacctgacccacagagtgcaccccatggcccccttcaacgtgttcgtgaagaatgtgtccgccaccaacgccaccatgacatggaaggtgcacagcatcggcaactactccaccctgctgtgtcagatcgagctggacggcgagggcaaagtgatccagaaacagaacgtgtcagtgaaagtgaacggcaagcacctgatgaagaagctggaacccagcaccgagtacgccgcccaggtgcgctgtgccaacgccaaccacttctggaagtggagtgaatggacccggcggaacttcaccacagccgaagccgccgctgagaacgaggtgtccacacctatgcaggccctgaccaccaacaaggacgacgacaacatcctgttccgggactccgccaatgccaccagcctgcctgtgcaggatagcagctctgtgctgcccgccaagcccgagaacatctcctgcgtgttctactacgaggaaaacttcacttgcacctggtcccccgagaaagaggccagctacacctggtacaaagtgaagagaacctacagctacggctacaagagcgacatctgccccagcgacaacagcaccagaggcaaccacaccttctgcagctttctgccccccaccatcaccaaccccgacaactacaccatccaggtggaagcccagaacgccgacggcatcatcaagtccgacatcacccactggtccctggacgccatcacaaagatcgagccccccgagatcttctccgtgaagcctgtgctgggcgtgaagaggatggtgcagatcaagtggatccggcccgtgctggccccagtgtctagcaccctgaagtacaccctgcggttcaagaccgtgaacagcgcctactggatggaagtgaatttcaccaaagaggacatcgaccgggacgagacatacaatctgaccggactgcaggccttcacagagtacgtgctggctctgagatgcgccaccaaagaatccatgttttggagcggctggtcccaggaaaagatgggcaccaccgaagagggtaagcctatccctaaccctctcctcggtctcgattctacgcgtaccggtcatcatcaccatcaccat

SEQ ID NO: 169 - protein, 474 aa, synthetic construct
  REGION 1..474: note = Amino acid sequence of feline IL-31-Ra fused to human IgG1 Fc
MMWPQVWGLEIQFSPQPACIDLGMMWAHALWTLLLLCKFSLAVLPAKPENISCVFYYEENFTCTWSPEKEASYTWYKVKRTYSYGYKSDICPSDNSTRGNHTFCSFLPPTITNPDNYTIQVEAQNADGIIKSDITHWSLDAITKIEPPEIFSVKPVLGVKRMVQIKWIRPVLAPVSSTLKYTLRFKTVNSAYWMEVNFTKEDIDRDETYNLTGLQAFTEYVLALRCATKESMFWSGWSQEKMGTTEEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - DNA, 1422 bp, synthetic construct
  misc_feature 1..1422: note = Nucleotide sequence encoding feline IL-31 Ra fused to human IgG1 Fc
  source 1..1422: mol_type = other DNA
atgatgtggccacaagtgtggggcctggagatccagttcagcccccagcctgcctgcatcgatctgggcatgatgtgggctcacgctctgtggaccctgctgctgctgtgcaagttttccctggccgtgctgcccgctaagcctgagaacatcagctgcgtgttctactatgaggagaacttcacctgtacatggtcccccgagaaggaggctagctatacctggtacaaggtgaagagaacatacagctatggctacaagtctgatatctgccccagcgacaactctacccgcggcaatcacacattctgttcttttctgccccctaccatcacaaaccctgataattataccatccaggtggaggcccagaacgctgatggcatcatcaagtctgacatcacccattggtccctggacgccatcacaaagatcgagccacccgagattttctccgtgaagcccgtgctgggcgtgaagaggatggtgcagatcaagtggatcaggcctgtgctggctccagtgtccagcaccctgaagtatacactgagattcaagaccgtgaactccgcttactggatggaggtgaacttcaccaaggaggacatcgatagggacgagacctataatctgacaggcctgcaggccttcaccgagtacgtgctggccctgaggtgcgctacaaaggagtccatgttttggtccggctggagccaggagaagatgggcaccacagaggaggataagacccacacatgccctccatgtccagctccagagctgctgggaggaccaagcgtgttcctgtttccacctaagcctaaggacaccctgatgatctctcgcacccctgaggtgacatgcgtggtggtggacgtgtcccacgaggacccagaggtgaagtttaactggtatgtggatggcgtggaggtgcataatgccaagaccaagcctagagaggagcagtataacagcacctaccgcgtggtgtctgtgctgacagtgctgcatcaggactggctgaacggcaaggagtacaagtgcaaggtgagcaataaggccctgcctgctccaatcgagaagaccatctctaaggctaagggacagccaagggagccacaggtgtatacactgccacccagccgggaggagatgaccaagaaccaggtgtctctgacatgtctggtgaagggcttctacccatctgatatcgctgtggagtgggagtccaatggccagcccgagaacaattataagaccacacctccagtgctggattctgacggctccttctttctgtactccaagctgaccgtggacaagagcaggtggcagcagggcaacgtgttttcttgctccgtgatgcatgaggctctgcacaatcattacacacagaagagcctgtctctgtccccaggcaag

SEQ ID NO: 171 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 172 - DNA, 1005 bp, Felis catus
  source 1..1005: mol_type = other DNA
gcctccaccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccggccgtcctgcaggcctcggggctgtactctctcagcagcatggtgacagtgccctccagcaggtggctcagtgacaccttcacctgcaacgtggcccacccgcccagcaacaccaaggtggacaagaccgtgcgcaaaacagaccacccaccgggacccaaaccctgcgactgtcccaaatgcccaccccctgagatgcttggaggaccgtccatcttcatcttccccccaaaacccaaggacaccctctcgatttcccggacgcccgaggtcacatgcttggtggtggacttgggcccagatgactccgatgtccagatcacatggtttgtggataacacccaggtgtacacagccaagacgagtccgcgtgaggagcagttcaacagcacctaccgtgtggtcagtgtcctccccatcctacaccaggactggctcaaggggaaggagttcaagtgcaaggtcaacagcaaatccctcccctcccccatcgagaggaccatctccaaggccaaaggacagccccacgagccccaggtgtacgtcctgcctccagcccaggaggagctcagcaggaacaaagtcagtgtgacctgcctgatcaaatccttccacccgcctgacattgccgtcgagtgggagatcaccggacagccggagccagagaacaactaccggacgaccccgccccagctggacagcgacgggacctacttcgtgtacagcaagctctcggtggacaggtcccactggcagaggggaaacacctacacctgctcggtgtcacacgaagctctgcacagccaccacacacagaaatccctcacccagtctccgggtaaa

SEQ ID NO: 173 - protein, 335 aa, synthetic construct
  REGION 1..335: note = Amino acid sequence of feline heavy chain engineered with modifications to modulate antibody effector function
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGAPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 174 - DNA, 1005 bp, synthetic construct
  misc_feature 1..1005: note = Nucleotide sequence encoding feline heavy chain engineered with modifications to modulate antibody effector function
  source 1..1005: mol_type = other DNA
gcctccaccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccggccgtcctgcaggcctcggggctgtactctctcagcagcatggtgacagtgccctccagcaggtggctcagtgacaccttcacctgcaacgtggcccacccgcccagcaacaccaaggtggacaagaccgtgcgcaaaacagaccacccaccgggacccaaaccctgcgactgtcccaaatgcccaccccctgaggcggctggagcaccgtccatcttcatcttccccccaaaacccaaggacaccctctcgatttcccggacgcccgaggtcacatgcttggtggtggacttgggcccagatgactccgatgtccagatcacatggtttgtggataacacccaggtgtacacagccaagacgagtccgcgtgaggagcagttcaacagcacctaccgtgtggtcagtgtcctccccatcctacaccaggactggctcaaggggaaggagttcaagtgcaaggtcaacagcaaatccctcccctcccccatcgagaggaccatctccaaggccaaaggacagccccacgagccccaggtgtacgtcctgcctccagcccaggaggagctcagcaggaacaaagtcagtgtgacctgcctgatcaaatccttccacccgcctgacattgccgtcgagtgggagatcaccggacagccggagccagagaacaactaccggacgaccccgccccagctggacagcgacgggacctacttcgtgtacagcaagctctcggtggacaggtcccactggcagaggggaaacacctacacctgctcggtgtcacacgaagctctgcacagccaccacacacagaaatccctcacccagtctccgggtaaa

SEQ ID NO: 175 - protein, 110 aa, synthetic construct
  REGION 1..110: note = Amino acid sequence of feline Kappa light chain engineered with glycosylation knockout (G-) at position 103
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFQRSECQRE

SEQ ID NO: 176 - DNA, 330 bp, synthetic construct
  misc_feature 1..330: note = Nucleotide sequence encoding feline Kappa light chain engineered with glyosylation knockout (G-)
  source 1..330: mol_type = other DNA
cggagtgatgctcagccatctgtctttctcttccaaccatctctggacgagttacatacaggaagtgcctctatcgtgtgcatattgaatgacttctaccccaaagaggtcaatgtcaagtggaaagtggatggcgtagtccaaaacaaaggcatccaggagagcaccacagagcagaacagcaaggacagcacctacagcctcagcagcaccctgacgatgtccagtacggagtaccaaagtcatgaaaagttctcctgcgaggtcactcacaagagcctggcctccaccctcgtcaagagcttccagaggagcgagtgtcagagagag

SEQ ID NO: 177 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 178 - DNA, 1005 bp, Canis sp.
  source 1..1005: mol_type = other DNA
gcctcaacaactgctcctagcgtgtttcccctggcccctagctgcggaagtacctcaggcagcacagtggccctggcttgtctggtgtctggatatttccctgagccagtgaccgtgagttggaacagcggctctctgacctccggggtgcacacatttccatctgtgctgcagtctagtggcctgtactccctgtcaagcatggtgactgtgccttcctctaggtggccatcagaaactttcacctgcaacgtggcccatcccgccagcaagaccaaagtggacaagcccgtgcctaaaagggagaatggaagggtgccaagaccacctgattgccctaagtgtccagctccagaagcggcgggagcaccaagcgtgttcatctttccacccaagcccaaagacacactgctgattgctagaactcccgaggtgacctgcgtggtggtggacctggatccagaggaccccgaagtgcagatctcctggttcgtggatgggaagcagatgcagacagccaaaactcagcctcgggaggaacagtttaacggaacctatagagtggtgtctgtgctgccaattggacaccaggactggctgaagggcaaacagtttacatgcaaggtgaacaacaaggccctgcctagtccaatcgagaggactatttcaaaagctaggggacaggctcatcagccttccgtgtatgtgctgcctccatcccgggaggaactgtctaagaacacagtgagtctgacttgtctgatcaaagatttctttccccctgacattgatgtggagtggcagagcaatgggcagcaggagccagaatccaagtacagaaccacaccaccccagctggacgaagatggctcctatttcctgtacagtaagctgtcagtggacaaatctaggtggcagcgcggggatacctttatctgcgccgtgatgcacgaggctctgcacaatcattacacacaagaaagtctgtcacatagccccggcaag

SEQ ID NO: 179 - protein, 106 aa, Canis sp.
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 180 - DNA, 318 bp, Canis sp.
  source 1..318: mol_type = other DNA
aggaacgacgcccagcctgctgtgtatctgtttcagccctcccctgatcagctgcacactggctctgctagtgtggtgtgtctgctgaacagcttctacccaaaggatatcaatgtgaagtggaaagtggacggcgtgatccaggatactgggattcaggagtccgtgaccgaacaggacaaagattcaacatatagcctgagctccactctgaccatgtctagtaccgagtacctgagccacgaactgtattcctgcgagatcactcataagtccctgccctctaccctgatcaagagcttccagagatcagagtgt

SEQ ID NO: 181 - protein, 164 aa, Homo sapiens
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 182 - protein, 111 aa, synthetic construct
  REGION 1..111: note = Caninized variable light chain mAb sequence, from Mus musculus and Canis
DIVMTQTPLSLSVSPGEPASISCRASESVDNYGISFMHWFQQKPGQSPQRLIYRASNLESGVPDRFSGSGSGTDFTLRISRVEADDAGVYYCQQSNKDPLTFGAGTKLEIK

SEQ ID NO: 183 - DNA, 333 bp, synthetic construct
  misc_feature 1..333: note = Nucleotide sequence encoding caninized variable light chain mAb sequence from Mus musculus and Canis
  source 1..333: mol_type = other DNA
gacatcgtgatgacccagacccccctgagcctgagcgtgtcccctggcgagcctgccagcatcagctgcagagccagcgagagcgtggacaactacggcatcagcttcatgcactggttccagcagaagcccggccagagcccccagcggctgatctacagagccagcaacctggaaagcggcgtgcccgatcggtttagcggctctggcagcggcaccgacttcaccctgcggatctctcgggtggaagccgatgacgccggagtgtactactgccagcagagcaacaaggaccccctgacctttggcgccggtaccaagctggagatcaag

SEQ ID NO: 184 - protein, 111 aa, synthetic construct
  REGION 1..111: note = Caninized variable light chain mAb sequence from Mus musculus and Canis
DIVMTQTPLSLSVSPGEPASISCRASESVDNYGISFMHWYQQKPGQPPKLLIYRASNLESGVPDRFSGSGSGTDFTLRISRVEADDAGVYYCQQSNKDPLTFGAGTKLEIK

SEQ ID NO: 185 - DNA, 333 bp, synthetic construct
  misc_feature 1..333: note = Nucleotide sequence encoding caninized variable light chain mAb sequence from Mus musculus and Canis
  source 1..333: mol_type = other DNA
gatatagtgatgacacaaactcctctcagtctttccgtatcaccgggagaaccggcttccatttcctgtcgggcctcagagtctgtggacaactacgggatatccttcatgcactggtatcagcagaaacccggccagccccctaaactccttatttacagggccagtaatctggaaagcggtgtgcccgatcgatttagcggttccgggagcggcacagatttcaccctgcgaatctctagagttgaagcggatgatgcaggagtatattactgccagcaatccaataaggatccccttacattcggcgcgggtaccaagctggagatcaag

SEQ ID NO: 186 - protein, 107 aa, synthetic construct
  REGION 1..107: note = Amino acid sequence of feline Kappa light chain G minus with modified C-terminus
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFQRSEC

SEQ ID NO: 187 - DNA, 324 bp, synthetic construct
  misc_feature 1..324: note = Nucleotide sequence encoding feline Kappa G minus with modified C-terminus
  source 1..324: mol_type = other DNA
cggagtgatgctcagccatctgtctttctcttccaaccatctctggacgagttacatacaggaagtgcctctatcgtgtgcatattgaatgacttctaccccaaagaggtcaatgtcaagtggaaagtggatggcgtagtccaaaacaaaggcatccaggagagcaccacagagcagaacagcaaggacagcacctacagcctcagcagcaccctgacgatgtccagtacggagtaccaaagtcatgaaaagttctcctgcgaggtcactcacaagagcctggcctccaccctcgtcaagagcttccagaggagcgagtgttag

SEQ ID NO: 188 - protein, 323 aa, Mus musculus
KTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 189 - DNA, 971 bp, Mus musculus
  source 1..971: mol_type = other DNA
ccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctggagtctgacctctacactctgagcagctcagtgactgtcccctccagccctcggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 190 - protein, 106 aa, Mus musculus
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 191 - DNA, 321 bp, Mus musculus
  source 1..321: mol_type = other DNA
gctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 192 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - DNA, 321 bp, Homo sapiens
  source 1..321: mol_type = other DNA
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 194 - protein, 109 aa, Canis sp.
NDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 195 - DNA, 330 bp, Canis sp.
  source 1..330: mol_type = other DNA
aatgatgcccagccagccgtctatttgttccaaccatctccagaccagttacacacaggaagtgcctctgttgtgtgcttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggactaa

SEQ ID NO: 196 - protein, 108 aa, Sus scrofa
ADAKPSVFIFPPSKEQLETQTVSVVCLLNSFFPREVNVKWKVDGVVQSSGILDSVTEQDSKDSTYSLSSTLSLPTSQYLSHNLYSCEVTHKTLASPLVKSFSRNECEA

SEQ ID NO: 197 - DNA, 327 bp, Sus scrofa
  source 1..327: mol_type = other DNA
gctgatgccaagccatccgtcttcatcttcccgccatcgaaggagcagttagagacccaaactgtctctgtggtgtgcttgctcaatagcttcttccccagagaagtcaatgtcaagtggaaagtggatggggtggtccaaagcagtggcatcctggatagtgtcacagagcaggacagcaaggacagcacctacagcctcagcagcaccctctcgctgcccacgtcacagtacctaagtcataatttatattcctgtgaggtcacccacaagaccctggcctcccctctggtcaaaagcttcagcaggaacgagtgtgaggcttag

SEQ ID NO: 198 - protein, 108 aa, Mustela vison
RNDAQPSVFLFQPSQDQLHTGSASVVCLLNGFYPKEVTVKWMVDGVTKNTGILESVTEQDSKDSTYSLSSTLTIPSTEYLSHETYSCEVTHKSLSSPLVKSFQRSECQ

SEQ ID NO: 199 - DNA, 327 bp, Mustela vison
  source 1..327: mol_type = other DNA
cggaatgatgcccagccatccgtctttttgttccaaccatctcaggaccagttacataccggcagtgcctctgtcgtgtgcttgctgaatggcttctaccccaaagaagtcactgtcaaatggatggttgatggtgttaccaaaaacacaggcatcctagaaagtgtcacagaacaagacagcaaggacagcacctacagcctcagcagcaccctgacgatccccagtacggagtacctaagtcatgagacgtactcctgtgaggtcactcacaagagcctgtcctcccctcttgtcaagagcttccaaaggagcgagtgccaatga

SEQ ID NO: 200 - protein, 5 aa, Mus musculus
SYWMN

SEQ ID NO: 201 - protein, 17 aa, Mus musculus
MIDPSDSEIHYNQVFKD

SEQ ID NO: 202 - protein, 9 aa, Mus musculus
QDIVTTVDY

SEQ ID NO: 203 - protein, 17 aa, Mus musculus
KSSQSLLYSINQKNHLA

SEQ ID NO: 204 - protein, 7 aa, Mus musculus
WASTRES

SEQ ID NO: 205 - protein, 9 aa, Mus musculus
QQGYTYPFT

SEQ ID NO: 206 - protein, 5 aa, Mus musculus
SYWMN

SEQ ID NO: 207 - protein, 17 aa, Mus musculus
MIDPSDSETHYNQIFRD

SEQ ID NO: 208 - protein, 9 aa, Mus musculus
QDIVTTVDY

SEQ ID NO: 209 - protein, 17 aa, Mus musculus
KSSQSLLYSINQKNFLA

SEQ ID NO: 210 - protein, 7 aa, Mus musculus
WASTRES

SEQ ID NO: 211 - protein, 9 aa, Mus musculus
QQHYGYPFT

SEQ ID NO: 212 - protein, 118 aa, Mus musculus
QVQLQQPGAELVRPGASVKLSCKASGYTFTSYWMNWAKQRPGQGLEWIGMIDPSDSEIHYNQVFKDKATLTVDKSSSTAYMQLSSLTSEDSAVYYCARQDIVTTVDYWGQGTTLTVSS

SEQ ID NO: 213 - DNA, 354 bp, Mus musculus
  source 1..354: mol_type = other DNA
caggtccaactgcagcagcctggggctgagctggtgaggcctggggcttcagtgaagctgtcctgcaaggcttctggctacaccttcaccagctactggatgaactgggcgaagcagaggcctggacaaggccttgaatggattggtatgattgatccttcagacagtgaaattcactacaatcaagtgttcaaggacaaggccacattgactgtagacaaatcctccagcacagcctacatgcagctcagcagcctgacatctgaggactctgcggtctattactgtgcaagacaagatatagtgactacagttgactactggggccaaggcaccactctcacagtctcctca

SEQ ID NO: 214 - protein, 113 aa, Mus musculus
DIVMSQSPSSLAVSVGEKVTMSCKSSQSLLYSINQKNHLAWFQQKPGQSPKLLIYWASTRESGVPARFTGSGSGTDFTLTISSVKTEDLAVYYCQQGYTYPFTFGSGTKLEIK

SEQ ID NO: 215 - DNA, 339 bp, Mus musculus
  source 1..339: mol_type = other DNA
gacattgtgatgtcacagtctccatcctccctagctgtgtcagttggagagaaggttacaatgagctgcaagtccagtcagagccttttatatagtatcaatcaaaagaaccacttggcctggttccagcagaaaccagggcagtctcctaaactgctgatttactgggcatccactagggaatctggggtccctgctcgcttcacaggcagtggatctggaacagatttcactctcaccatcagcagtgtgaagactgaagacctggcagtttattactgtcagcaaggttatacctacccattcacgttcggctcggggacaaagttggaaataaaa

SEQ ID NO: 216 - protein, 118 aa, Mus musculus
QVQLQQPGAELVRPGASVKLSCKAYGYTFTSYWMNWVKQRPGQGLEWIGMIDPSDSETHYNQIFRDKATLTIDKSSSTAYMQLSSLTSEDSAVYFCARQDIVTTVDYWGQGTTLTVSS

SEQ ID NO: 217 - DNA, 354 bp, Mus musculus
  source 1..354: mol_type = other DNA
caggtccaattgcagcagcctggggctgagctggtgaggcctggggcttcagtgaagctgtcctgcaaggcctatggctacaccttcaccagttactggatgaactgggtgaaacagaggcctggacaaggccttgaatggattggtatgattgatccttcagacagtgaaactcactacaatcaaatattcagggacaaggccacattgactatagacaaatcctccagcacagcctacatgcagctcagcagcctgacatctgaggactctgcggtctatttctgtgcaagacaagatatagtgactacagttgactactggggccagggcaccactctcacagtctcctca

SEQ ID NO: 218 - protein, 113 aa, Mus musculus
DIVMSQSPSSLAVSVGEKVTMSCKSSQSLLYSINQKNFLAWYQQKPGQSPKLLIYWASTRESGVPDRFTGSGSGTDFTLTISSVKSEDLAVYYCQQHYGYPFTFGSGTKLEIK

SEQ ID NO: 219 - DNA, 339 bp, Mus musculus
  source 1..339: mol_type = other DNA
gacattgtgatgtcacagtctccatcctccctagctgtgtcagttggagagaaggttactatgagctgcaagtccagtcagagccttttatatagtatcaatcaaaagaacttcttggcctggtaccagcagaaaccagggcagtctcctaaactgctgatttactgggcatccactagggaatctggggtccctgatcgcttcacaggcagtggatctgggacagatttcactctcaccatcagcagtgtgaagtctgaagacctggcagtttattactgtcagcaacattatggctatccattcacgttcggctcggggacaaagttggaaataaaa

SEQ ID NO: 220 - protein, 118 aa, synthetic construct
  REGION 1..118: note = Felinized variable heavy mAb sequence, from Mus musculus and Felis catus
DVQLVESGGDLVKPGGSLRLTCVASGFTYSNYWMHWVRQAPGKGLQWVARIDPYGGGTKHNEKFKRRFTISRDNAKNTLYLQMNSLKTEDTATYYCVRSGYDYYFDVWGQGTLVTVSS

SEQ ID NO: 221 - DNA, 354 bp, synthetic construct
  misc_feature 1..354: note = Nucleotide sequence encoding felinized variable heavy mAb sequence, from Mus musculus and Felis catus
  source 1..354: mol_type = other DNA
gacgtgcaactggtggagagtggaggggaccttgtaaagccaggcgggtccctgcgcctgacctgtgtagccagcggcttcacttactccaactattggatgcactgggtcagacaggcccccggaaaagggcttcagtgggtggcaaggatcgatccctacggaggaggaacgaagcataacgagaagttcaagcggaggtttactatcagtagagacaacgcgaaaaatacactgtacctgcagatgaatagtcttaagacagaggataccgcgacctactattgcgtcagatccggctatgactattactttgacgtttggggacagggcacactggtcaccgtctcgagc

SEQ ID NO: 222 - protein, 107 aa, synthetic construct
  REGION 1..107: note = Felinized variable light mAb sequence, from Mus musculus and Felis catus
EIQMTQSPSSLSASPGDRVTITCRASENIYSFLAWYQQKPGKVPKLLIYNANTLAEGVPSRFSGSGSGTDFTLTISSLEPEDAATYYCQHHFGTPFTFGSGTKLEIK

SEQ ID NO: 223 - protein, 321 aa, synthetic construct
  REGION 1..321: note = Nucleotide sequence encoding felinized variable light mAb sequence, from Mus musculus and Felis catus
GAAATTCAAATGACCCAGTCACCCAGTTCCTTGTCCGCCAGTCCCGGAGATCGCGTCACCATAACCTGCCGGGCCAGTGAAAATATTTATTCATTCCTCGCGTGGTATCAGCAGAAGCCAGGCAAGGTGCCGAAGCTGCTGATATACAACGCTAACACGTTGGCCGAGGGGGTGCCCAGTAGATTCAGCGGGTCCGGCAGTGGAACCGATTTCACCCTGACAATTTCAAGCCTGGAACCAGAAGACGCCGCCACATACTACTGCCAGCATCACTTTGGGACCCCTTTCACTTTCGGATCCGGTACCAAGCTGGAGATCAAA

SEQ ID NO: 224 - protein, 118 aa, synthetic construct; identical to SEQ ID NO: 220
  REGION 1..118: note = Felinized variable heavy mAb sequence, from Mus musculus and Felis catus

SEQ ID NO: 225 - DNA, 354 bp, synthetic construct; identical to SEQ ID NO: 221
  misc_feature 1..354: note = Nucleotide sequence encoding felinized variable heavy mAb sequence, from Mus musculus and Felis catus
  source 1..354: mol_type = other DNA

SEQ ID NO: 226 - protein, 107 aa, synthetic construct; identical to SEQ ID NO: 222
  REGION 1..107: note = Felinized variable light mAb sequence, from Mus musculus and Felis catus

SEQ ID NO: 227 - protein, 321 aa, synthetic construct; identical to SEQ ID NO: 223
  REGION 1..321: note = Nucleotide sequence encoding felinized variable light mAb sequence, from Mus musculus and Felis catus

SEQ ID NO: 228 - protein, 117 aa, synthetic construct
  REGION 1..117: note = Felinized variable heavy mAb sequence, from Canis familiaris and Felis catus
DVQLVESGGDLVKPGGSLRLTCVASGFTFSDYAMSWVRQAPGKGLQWVAGIDSVGSGTSYADSVKGRFTISRDNAKNTLYLQMNSLKTEDTATYYCASGFPGSFEHWGQGALVTVSS

SEQ ID NO: 229 - DNA, 351 bp, synthetic construct
  misc_feature 1..351: note = Nucleotide sequence encoding felinized variable heavy mAb sequence, Canis familiaris and Felis catus
  source 1..351: mol_type = other DNA
gatgtgcagctggtggaaagcggcggcgatctggtgaaaccgggcggcagcctgcgcctgacctgcgtggcgagcggctttacctttagcgattatgcgatgagctgggtgcgccaggcgccgggcaaaggcctgcagtgggtggcgggcattgatagcgtgggcagcggcaccagctatgcggatagcgtgaaaggccgctttaccattagccgcgataacgcgaaaaacaccctgtatctgcagatgaacagcctgaaaaccgaagataccgcgacctattattgcgcgagcggctttccgggcagctttgaacattggggccagggcgcgctggtgaccgtgagcagc

SEQ ID NO: 230 - protein, 132 aa, synthetic construct
  REGION 1..132: note = Felinized variable light mAb sequence, from Canis familiaris and Felis catus
QSVLTQPSSVSGTLGQRITISCTGSSSNIGSGYVGWYQQVPGMGPKTVIYYNSDRPSGVPDRFSGSKSGSSGTLTITGLQAEDEADYYCSVYDRTFNAVFGGGTHLTVLGQPKSAPPRSHSSRPISYAVFCL

SEQ ID NO: 231 - DNA, 327 bp, synthetic construct
  misc_feature 1..327: note = Nucleotide sequence encoding felinized variable light mAb sequence, Canis familiaris and Felis catus
  source 1..327: mol_type = other DNA
cagagcgtgctgacccagccgagcagcgtgagcggcaccctgggccagcgcattaccattagctgcaccggcagcagcagcaacattggcagcggctatgtgggctggtatcagcaggtgccgggcatgggcccgaaaaccgtgatttattataacagcgatcgcccgagcggcgtgccggatcgctttagcggcagcaaaagcggcagcagcggcaccctgaccattaccggcctgcaggcggaagatgaagcggattattattgcagcgtgtatgatcgcacctttaacgcggtgtttggcggcggcacccatctgaccgtgctg

SEQ ID NO: 232 - protein, 117 aa, synthetic construct
  REGION 1..117: note = Felinized variable heavy mAb sequence, from Canis familiaris and Felis catus
DVQLVESGGDLVKPGGSLRLTCVASGFTFSDYAMNWVRQAPGKGLQWVAGIDSVGSGTSYADSVKGRFTISRDNAKNTLYLQMSGLKTEDTATYYCASGFPGSFEHWGQGTLVTVSS

SEQ ID NO: 233 - DNA, 351 bp, synthetic construct
  misc_feature 1..351: note = Nucleotide sequence encoding felinized variable heavy mAb sequence, Canis familiaris and Felis catus
  source 1..351: mol_type = other DNA
gatgtgcagctggtggaaagcggcggcgatctggtgaaaccgggcggcagcctgcgcctgacctgcgtggcgagcggctttacctttagcgattatgcgatgaactgggtgcgccaggcgccgggcaaaggcctgcagtgggtggcgggcattgatagcgtgggcagcggcaccagctatgcggatagcgtgaaaggccgctttaccattagccgcgataacgcgaaaaacaccctgtatctgcagatgagcggcctgaaaaccgaagataccgcgacctattattgcgcgagcggctttccgggcagctttgaacattggggccagggcaccctggtgaccgtgagcagc

SEQ ID NO: 234 - protein, 132 aa, synthetic construct; identical to SEQ ID NO: 230
  REGION 1..132: note = Felinized variable light mAb sequence, from Canis familiaris and Felis catus

SEQ ID NO: 235 - DNA, 327 bp, synthetic construct; identical to SEQ ID NO: 231
  misc_feature 1..327: note = Nucleotide sequence encoding felinized variable light mAb sequence, Canis familiaris and Felis catus
  source 1..327: mol_type = other DNA

SEQ ID NO: 236 - protein, 106 aa, Felis catus
GQPKSAPSVTLFPPSNEELSANKATLVCLISDFYPSGLTVAWKADGTPITQGVETTKPSKQSNNKYAASSYLSLSPNEWKSRSRFTCQVTHEGSTVEKNVVPAECS

SEQ ID NO: 237 - DNA, 318 bp, Felis catus
  source 1..318: mol_type = other DNA
ggccagcccaagagcgctccctccgtgaccctgttccccccaagcaacgaggaactgagcgccaacaaggccaccctggtgtgcctgatcagcgacttctaccccagcggcctgaccgtggcctggaaggccgatggcacccctatcacccagggcgtggaaaccaccaagcccagcaagcagagcaacaacaaatacgccgccagcagctacctgagcctgagccccaacgagtggaagtcccggtcccggttcacatgccaggtgacacacgagggcagcaccgtggaaaagaacgtggtgcccgccgagtgcagc